(19) United States
(12) Patent Application Publication
(10) Pub. No.: US 2025/0051477 A1
(43) Pub. Date: Feb. 13, 2025

(54) ANTI-MUSK ANTIBODIES FOR USE IN TREATING NEUROMUSCULAR DISORDERS

(71) Applicants: argenx BV, Ghent (BE); Université De Montréal, Montreal (CA); New York University, New York, NY (US)

(72) Inventors: Roeland Vanhauwaert, Ghent (BE); Karen Silence, Ghent (BE); Richard Robitaille, Montreal (CA); Danielle Arbour, Montreal (CA); Laurence Renaud, Montreal (CA); Steven J. Burden, New York, NY (US)

(21) Appl. No.: 18/782,814

(22) Filed: Jul. 24, 2024

Related U.S. Application Data
(63) Continuation of application No. PCT/US2023/061476, filed on Jan. 27, 2023.
(60) Provisional application No. 63/364,685, filed on May 13, 2022.

(30) Foreign Application Priority Data
Jan. 28, 2022 (EP) 22154118.8

(51) Int. Cl. C07K 16/40; A61K 39/00; A61P 21/00

(52) U.S. Cl. CPC C07K 16/40; A61P 21/00; A61K 2039/505; C07K 2317/52; C07K 2317/565; C07K 2317/71

(57) ABSTRACT

The present invention relates to an anti-MuSK antibody or antigen binding fragment thereof for use in the treatment of a neuromuscular disorder in a human subject. In an embodiment, this antibody or antigen binding fragment thereof is combined with an anticholinergic compound.

### RELATED APPLICATIONS

This application is a continuation of International Application No. PCT/US2023/061476, filed Jan. 27, 2023, which claims priority to U.S. Provisional Patent Application Ser. No. 63/364,685, filed May 13, 2022, and EP Application No. 22154118.8, filed Jan. 28, 2022, the entire disclosure of which is hereby incorporated herein by reference

### REFERENCE TO SEQUENCE LISTING

This application contains a sequence listing which has been submitted electronically in ST.26 format and is hereby incorporated by reference in its entirety (said ST.26 copy, created Jan. 27, 2023, is named “196198_SL.XML” and is 364,704 bytes in size)

### FIELD OF THE INVENTION

The present invention relates to an anti-MuSK antibody or antigen binding fragment thereof for use in the treatment of a neuromuscular disorder such as ALS (amyotrophic lateral sclerosis) in a human subject. In an embodiment, this antibody or antigen binding fragment thereof is combined with an anticholinergic compound.

### BACKGROUND

ALS is an adult-onset non-cell autonomous neuromuscular/neurodegenerative disorder which causes the progressive loss of upper and lower motor neurons (MN), leading to gradual paralysis and death in 2 to 5 years. Neuromuscularjunction (NMJ) denervation is a hallmark of ALS [1] and is present in several disease models of ALS [2-6], even preceding the death of MN [1, 3].

The currently approved ALS treatment (Riluzole) benefits only 20% of ALS patients by extending their life for approximately three months. The effect of Riluzole on muscle function is very limited. Moreover, ALS is considered a genetically heterogenous disease likely representing several subgroups with differing underlying pathology. There is currently no cure available, nor will patient-tailored therapies likely be able to aid all ALS patients because of the different underlying disease mechanisms

Therefore, there is still a need for a new therapy for ALS and other diseases that mimic ALS (‘ALS-like diseases’).

### LEGEND TO THE DRAWINGS

FIGS. 1A-1E Combo treatment ameliorates locomotor functions in SOD1G37R mice. ARGX-119 treatment was started at P400 (disease pre-onset or asymptomatic) and darifenacin treatment was started at ˜P425 (disease onset) and continued until sacrifice (˜P520). FIG. 1A) Diagram depicting the Rotarod apparatus used to assess motor function, coordination and balance following an acceleration of the rotating wheel. FIG. 1B) Latency to fall (sec) on the Rotarod of ARGX-119 antibody (big gray circle), Combo treatment of ARGX-119+darifenacin treated mice (black triangle), darifenacin-treated mice (small gray circle) versus double placebo-treated mice (gray square) between ˜400 until 520 days of age. FIG. 1C) Grip strength meter used to monitor the overall strength of mice forelimbs and hindlimbs. FIG. 1D) Evolution of grip strength measurements during the course of the treatment, from ˜P400 to P520 for the groups. FIG. 1E) Evolution of body weight measurements during the course of the treatment, from ˜P400 to P520 for the groups. * p<0.05, *** p<0.001, **** p<0.0001. One-way ANOVA and multiple t-test.

FIGS. 2A-2D Treatment improves the contractile properties of the EDL muscle. FIG. 2A) Picture of the set up of the muscle force transducer and the nerve and muscle stimulating electrodes used to evoke muscle contractions. Examples of raw data show a muscle contraction elicited by nerve or muscle stimulation, used to calculate the contractile capacity ratio. FIGS. 2B-2C) Peak twitch force of the EDL muscle generated by nerve stimulations (FIG. 2B) or muscle stimulation at different frequencies (5 Hz-300 Hz) (FIG. 2C) from ARGX-119 antibody (gray circle) and Combo treatment of ARGX-119+darifenacin treated mice (black triangle) versus double placebo-treated mice (gray square). FIG. 2D) Histogram showing the mean±SEM of the contractile capacity ratio expressed in percentage for the EDL muscle, representing the proportion of the peak force generated by nerve stimulation over muscle stimulation (stimulation frequencies between 5 Hz-100 Hz). FIG. 2E) Histogram showing the mean±SEM of EDL muscle weight from ARGX-119 antibody, combo-treated and placebo-treated mice. * p<0.05, ** p<0.01, *** p<0.001, **** p<0.0001. Repeated one-way ANOVA and multiple t-test.

FIGS. 3A-3D Combo-treatment improves contractile properties of the Soleus muscle. FIGS. 3A-3B) Peak twitch force of the Soleus (SOL) muscle generated by nerve stimulations (FIG. 3A) or muscle stimulation at different frequencies (5 Hz-300 Hz) (FIG. 3B) ARGX-119 antibody (gray circle) and Combo treatment of ARGX-119+darifenacin treated mice (black triangle) versus double placebo-treated mice (gray square). FIG. 3C) Histogram showing the mean±SEM of the contractile capacity ratio expressed in percentage for the SOL muscle, representing the proportion of the peak force generated by nerve stimulation over muscle stimulation (stimulation frequencies between 5 Hz-100 Hz). (FIG. 3D) Histogram showing the mean±SEM of EDL muscle weight from ARGX-119 antibody, combo-treated and placebo-treated mice. * p<0.05, ** p<0.01, *** p<0.001, **** p<0.0001. Repeated one-way ANOVA and multiple t-test.

FIGS. 4A-4E Combo-treatment preserves muscle fatigue properties. FIG. 4A) Diagram illustrating the EDL fatigue protocol, which consists of 18 trains of 10 stimulations elicited at 120 Hz for 300 ms (1 train per second). Nerve stimulations alone are used 9 out of 10 bouts and muscle stimulation is superimposed to the nerve stimulation every 10 stimulations. The fatigue protocol is followed by a 30-minute recovery period. FIGS. 4B-4C) Peak contractile force during the fatigue protocol and the recovery period expressed as the percentage of the initial baseline force generated before the fatigue protocol, for nerve stimulation (FIG. 4B) and nerve+muscle (FIG. 4C) of the EDL muscle. Note the higher resistance to fatigue in placebo-treated (gray square) compared to double-treated ARGX-119+darifenacin animals (black triangle) and ARGX-119 antibody-treated (gray circle). This illustrates a significant alteration of the normal fast-twitch muscle properties that is normally highly fatigable. FIGS. 4D-4E) Peak contractile force during the fatigue protocol and the recovery period expressed as the percentage of the initial baseline force generated before the fatigue protocol, for nerve stimulation (FIG. 4D) and nerve+muscle (FIG. 4E) of the SOL muscle. *, p<0.05, ** p<0.01, *** p<0.001, **** p<0.0001. Repeated one-way ANOVA and multiple t-test.

### DETAILED DESCRIPTION OF THE INVENTION

### General Definitions

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms “a”, “an”, and “the” include both singular and plural referents unless the context clearly dictates otherwise.

The terms “comprising”, “comprises” and “comprised of” as used herein are synonymous with ‘including’, ‘includes’ or ‘containing’, ‘contains’, and are inclusive or open-ended and do not exclude additional, non-recited members, compounds, products, elements or method steps. The expression “essentially consists of” used in the context of a product or a composition (“a product essentially consisting of” or “a composition essentially consisting of”) means that additional molecules may be present but that such molecule does not change/alter the characteristic/activity/functionality of said product or composition. For example, a composition may essentially consist of an antibody or an antibody fragment if the composition as such would exhibit similar characteristic/activity/functionality as one of the antibody or as the one of the antibody fragments.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The terms ‘disorder’ and ‘disease’ are used herein interchangeably.

As used herein, amino acid residues will be indicated either by their full name or according to the standard three-letter or one-letter amino acid code.

As used herein, the terms “polypeptide” or “protein” are used interchangeably, and refer to a polymeric form of amino acids of any length, which can include coded and non-coded amino acids, chemically or biochemically modified or derivatized amino acids, and polypeptides having modified peptide backbones. A “peptide” is also a polymer of amino acids with a length which is usually of up to 50 amino acids. A polypeptide or peptide is represented by an amino acid sequence.

As used herein, the terms “nucleic acid molecule”, “polynucleotide”, “polynucleic acid”, “nucleic acid” are used interchangeably and refer to polymeric form of nucleotides of any length, either deoxyribonucleotides or ribonucleotides, or analogs thereof. A nucleic acid molecule is represented by a nucleic acid sequence, which is primarily characterized by its base sequence. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “homology” denotes at least secondary structural identity or similarity between two macromolecules, particularly between two polypeptides or polynucleotides, from same or different taxons, wherein said similarity is due to shared ancestry. Hence, the term ‘homologues’ denotes so-related macromolecules having said secondary and optionally tertiary structural similarity. For comparing two or more nucleotide sequences, the ‘(percentage of) sequence identity’ between a first nucleotide sequence and a second nucleotide sequence may be calculated using methods known by the person skilled in the art, e.g. by dividing the number of nucleotides in the first nucleotide sequence that are identical to the nucleotides at the corresponding positions in the second nucleotide sequence by the total number of nucleotides in the first nucleotide sequence and multiplying by 100% or by using a known computer algorithm for sequence alignment such as NCBI Blast. In determining the degree of sequence similarity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions have been already exemplified herein. Amino acid sequences and nucleic acid sequences are said to be ‘exactly the same’ if they have 100% sequence identity over their entire length.

Throughout this application, each time one refers to a specific amino acid sequence SEQ ID NO (take SEQ ID NO: Y as example), one may replace it by: a polypeptide comprising an amino acid sequence that has at least 80% sequence identity or similarity with amino acid sequence SEQ ID NO: Y. Throughout this application, the wording “a sequence is at least X % identical with another sequence” may be replaced by “a sequence has at least X % sequence identity with another sequence”.

Each amino acid sequence described herein by virtue of its identity percentage (at least 80%) with a given amino acid sequence respectively has in a further preferred embodiment an identity of at least 80%, 81%, 82%, 83%, 84%, 85%, 86%, 87%, 88%, 89%, 90%, 91%, 92%, 93%, 94%, 95%, 96%, 97%, 98%, 99% or more identity with the given amino acid sequence respectively. In a preferred embodiment, sequence identity is determined by comparing the whole length of the sequences as identified herein. Each amino acid sequence described herein by virtue of its similarity percentage (at least 80%) with a given amino acid sequence respectively has in a further preferred embodiment a similarity of at least 80%, 81%, 82%, 83%, 84%, 85%, 86%, 87%, 88%, 89%, 90%, 91%, 92%, 93%, 94%, 95%, 96%, 97%, 98%, 99% or more similarity with the given amino acid sequence respectively. In a preferred embodiment, sequence similarity is determined by comparing the whole length of the sequences as identified herein. Unless otherwise indicated herein, identity or similarity with a given SEQ ID NO means identity or similarity based on the full length of said sequence (i.e. over its whole length or as a whole). “Sequence identity” is herein defined as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, “identity” also means the degree of sequence relatedness between amino acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs.

Preferred computer program methods to determine identity and similarity between two sequences include e.g. the GCG program package (Devereux, J., et al., Nucleic Acids Research 12 (1): 387 (1984)), BestFit, FASTA, BLASTN, and BLASTP (Altschul, S. F. et al., J. Mol. Biol. 215:403-410 (1990)), EMBOSS Needle (Madeira, F., et al., Nucleic Acids Research 47(W1): W636-W641 (2019)). The BLAST program is publicly available from NCBI and other sources (BLAST Manual, Altschul, S., et al., NCBI NLM NIH Bethesda, MD 20894; Altschul, S., et al., J. Mol. Biol. 215:403-410 (1990)). The EMBOSS program is publicly available from EMBL-EBI. The well-known Smith Waterman algorithm may also be used to determine identity. The EMBOSS Needle program is the preferred program used.

Preferred parameters for polypeptide sequence comparison include the following: Algorithm: Needleman and Wunsch, J. Mol. Biol. 48 (3):443-453 (1970); Comparison matrix: BLOSUM62 from Henikoff and Henikoff, Proc. Natl. Acad. Sci. USA. 89:10915-10919 (1992); Gap Open Penalty: 10; and Gap Extend Penalty: 0.5. A program useful with these parameters is publicly available as the EMBOSS Needle program from EMBL-EBI. The aforementioned parameters are the default parameters for a Global Pairwise Sequence alignment of proteins (along with no penalty for end gaps).

Preferred parameters for nucleic acid comparison include the following: Algorithm: Needleman and Wunsch, J. Mol. Biol. 48:443-453 (1970); Comparison matrix: DNAfuII; Gap Open Penalty: 10; Gap Extend Penalty: 0.5. A program useful with these parameters is publicly available as the EMBOSS Needle program from EMBL-EBI. The aforementioned parameters are the default parameters for a Global Pairwise Sequence alignment of nucleotide sequences (along with no penalty for end gaps).

As used herein, the terms “disorder” and “disease” are used interchangeably.

Also provided herein are embodiments wherein any embodiment described herein may be combined with any one or more other embodiments, provided the combination is not mutually exclusive.

### Anti-MuSK Antibody or Antigen-Binding Fragment Thereof

All antibodies or antigen-binding fragments thereof defined herein are encompassed as such in the present invention. The antibodies or antigen-binding fragments thereof are also for use in the treatment of a neuromuscular disorder in a human subject.

The present invention relates to anti-MuSK antibody-based molecules, including anti-MuSK antibodies, epitope-binding domains thereof, antigen binding fragments thereof and antibody derivatives that are for treating a neuromuscular disease or condition. In an embodiment, the wording “antibody-based molecule” may be replaced by the word “antibody” or by the expression “antibody or a functional fragment thereof” or by the expression “antibody or antigen binding fragment”.

The term “anti-MuSK antibody” may be replaced by the term “MuSK antibody”.

Any anti-MuSK antibody-based molecule including anti-MuSK antibodies, epitope-binding domains thereof, antigen binding fragments thereof and antibody derivatives that is capable of binding muscle-specific tyrosine protein kinase (MuSK) is encompassed within the present invention. In an embodiment, such anti-MuSK antibody is also able to activate the signaling and/or phosphorylation of MuSK. The invention provides the insight that such antibody-based molecules are useful for the treatment of conditions where a subject is in need of increased MuSK signaling or MuSK phosphorylation, such as neuromuscular disease or conditions. Therefore in a first aspect, there is provided an anti-MuSK antibody or antigen-binding fragment thereof for use in the treatment of a neuromuscular disorder in a human subject.

MuSK is a receptor tyrosine kinase that is expressed in skeletal muscle and has a crucial, master role in forming and maintaining neuromuscular synapses (Burden et al., “The Role of MuSK in Synapse Formation and Neuromuscular Disease,” Cold Spring Harb. Perspect. Biol. 5:a009167 (2013), which is hereby incorporated by reference in its entirety). MuSK is a single pass, 120 kDa transmembrane protein, composed of an extracellular region containing three Ig-like domains and a Frizzled (Fz)-like domain, and an intracellular region containing a juxtamembrane region, a kinase domain and a short cytoplasmic tail (Jennings et al., “Muscle-Specific trk-Related Receptor with a Kringle Domain Defines a Distinct Class of Receptor Tyrosine Kinases,” Proc. Natl. Acad. Sci. USA 90:2895-2899 (1993) and Valenzuela et al., “Receptor Tyrosine Kinase Specific for the Skeletal Muscle Lineage: Expression in Embryonicmuscle, at the Neuromuscular Junction, and After Injury,” Neuron 15: 573-584 (1995), which are hereby incorporated by reference in their entirety). MuSK phosphorylation is stimulated by agrin, a signal provided by motor neurons. Once activated, MuSK stimulates pathways that (1) cluster and anchor AChRs and additional muscle proteins critical for synaptic transmission, (2) enhance transcription of genes encoding synaptic proteins in muscle ‘synaptic nuclei’ and (3) promote the production of retrograde signals that promote presynaptic differentiation and attachment of motor nerve terminals to muscle. In the absence of MuSK, neuromuscular synapses fail to form (Burden et al., “The Role of MuSK in Synapse Formation and Neuromuscular Disease,” Cold Spring Harb. Perspect. Biol. 5:a009167 (2013), which is hereby incorporated by reference in its entirety). In addition to its role during synapse formation, MuSK is also required to maintain adult synapses, as inhibition of MuSK expression in adult muscle leads to profound defects in presynaptic and postsynaptic differentiation (Kong et al., “Inhibition of Synapse Assembly in Mammalian Muscle in vivo by RNA Interference,” EMBO Rep 5:183-188 (2004) and Hesser et al., “Synapse Disassembly and Formation of New Synapses in Postnatal Muscle Upon Conditional Inactivation of MuSK,” Mol. Cell. Neurosci. 31:470-480 (2006), which are hereby incorporated by reference in their entirety). Consistent with these findings in mice, mutations that impair MuSK kinase activity or inhibit signaling steps downstream from MuSK cause myasthenia (CM), characterized by structurally and functionally defective synapses, leading to muscle weakness and fatigue (Beeson et al., “Dok-7 Mutations Underlie a Neuromuscular Junction Synaptopathy,” Science 313:1975-1978 (2006); Muller et al., “Phenotypical Spectrum of DOK7 Mutations in Congenital Myasthenic Syndromes,” Brain 130:1497-1506 (2007); and Selcen et al., “A Compensatory Subpopulation of Motor Neurons in a Mouse Model of Amyotrophic Lateral Sclerosis,” J. Comp. Neurol. 490:209-219 (2008), which are hereby incorporated by reference in their entirely).

The amino acid sequence of human MuSK has the amino acid sequence of SEQ ID NO: 129 below.

(SEQ ID NO: 129)
MRELVNIPLVHILTLVAFSGTEKLPKAPVITTPLETVDALVEEVATFMCAVESYPQPEISWTRNKILIKLF
DTRYSIRENGQLLTILSVEDSDDGIYCCTANNGVGGAVESCGALQVKMKPKITRPPINVKIIEGLKAVLP
CTTMGNPKPSVSWIKGDSPLRENSRIAVLESGSLRIHNVQKEDAGQYRCVAKNSLGTAYSKVVKLEV
EVFARILRAPESHNVTFGSFVTLHCTATGIPVPTITWIENGNAVSSGSIQESVKDRVIDSRLQLFITKPGL
YTCIATNKHGEKFSTAKAAATISIAEWSKPQKDNKGYCAQYRGEVCNAVLAKDALVFLNTSYADPEEA
QELLVHTAWNELKVVSPVCRPAAEALLCNHIFQECSPGVVPTPIPICREYCLAVKELFCAKEWLVMEE
KTHRGLYRSEMHLLSVPECSKLPSMHWDPTACARLPHLDYNKENLKTFPPMTSSKPSVDIPNLPSSS
SSSFSVSPTYSMTVIISIMSSFAIFVLLTITTLYCCRRRKQWKNKKRESAAVTLTTLPSELLLDRLHPNPM
YQRMPLLLNPKLLSLEYPRNNIEYVRDIGEGAFGRVFQARAPGLLPYEPFTMVAVKMLKEEASADMQA
DFQREAALMAEFDNPNIVKLLGVCAVGKPMCLLFEYMAYGDLNEFLRSMSPHTVCSLSHSDLSMRAQ
VSSPGPPPLSCAEQLCIARQVAAGMAYLSERKFVHRDLATRNCLVGENMVVKIADFGLSRNIYSADYY
KANENDAIPIRWMPPESIFYNRYTTESDVWAYGVVLWEIFSYGLQPYYGMAHEEVIYYVRDGNILSCP
ENCPVELYNLMRLCWSKLPADRPSFTSIHRILERMCERAEGTVSV

In accordance with the present invention, the MuSK antibody-based molecules described herein bind to an epitope within the Frizzled (Fz)-like domain of the MuSK protein. The Fz-like domain of MuSK has the amino acid sequence of SEQ ID NO: 130 as shown below.

(SEQ ID NO: 130)
DNKGYCAQYRGEVCNAVLAKDALVFLNTSYADPEEAQELLVHTAWNELK
VVSPVCRPAAEALLCNHIFQECSPGVVPTPIPICREYCLAVKELFCAKE
WLVMEEKTHRGLYRSEMHLLSVPECSKLPSMHWDPTACARL

The term “epitope” as used herein refers to an antigenic determinant capable of being bound to an antibody. Epitopes usually comprise surface groupings of molecules such as amino acids or sugar side chains and usually have specific three dimensional structural characteristics, as well as specific charge characteristics. Conformational and non-conformational epitopes are distinguished in that the binding to the former, but not the latter, is lost in the presence of denaturing solvents. An epitope may comprise amino acid residues directly involved in the binding (also called the immunodominant component of the epitope) and other amino acid residues, which are not directly involved in the binding, such as amino acid residues that are effectively blocked by the specific antigen-binding peptide (in other words, the amino acid residue is within the footprint of the specific antigen-binding peptide). An epitope typically includes at least 3, and more usually, at least 5, 6, 7, 8, 9, 10, 11, 12, 13, 14, 15, 16, 17, 18, 19, 20 or more amino acids in a unique spatial conformation.

In an embodiment, the MuSK antibody or antigen binding fragment, for use according to the invention binds the MuSK Frizzled (Fz) like domain. In an embodiment, the MuSK antibody or antigen binding fragment immunospecifically bind an epitope within the MuSK Fz-like domain sequence of SEQ ID NO: 130 more frequently, more rapidly, with greater duration and/or with greater affinity or avidity than an alternative epitope. In an embodiment, the MuSK antibody-based molecules described herein bind immunospecifically to any 2, 3, 4, 5, 6, 7, 8, 9, 10 or more amino acid residues of SEQ ID NO: 130. The term “affinity”, “specific binding”, “binding”, “immunospecific binding”, “binding activity” or “specific binding activity”, as used herein, refers to the degree to which an antibody or an antibody fragment as defined herein binds to an epitope within the MuSK-Fz-like domain sequence of SEQ ID NO:130.

In an embodiment, the MuSK antibody-based molecules as disclosed herein bind to the MuSK Fz-like domain with an affinity corresponding to a KD of about 10-7 M or less. For example, the MuSK antibody-based molecules disclosed herein bind to the MuSK Fz-like domain with an affinity corresponding to a KD of about 10-8 M, of about 10-9 M, of about 10-10 M, of about 10-11 M, of about 10-12 M or less when determined by, for instance, surface plasmon resonance (SPR) technology in a Biacore 3000 instrument (preferably using the antibody as the ligand and MuSK as the analyte). The MuSK antibody-based molecules as disclosed herein bind to the MuSK Fz-like domain with an affinity corresponding to a KD that is at least ten-fold lower, such as at least 100 fold lower, for instance at least 1,000 fold lower, such as at least 10,000 fold lower, for instance at least 100,000 fold lower than its affinity for binding to a non-specific antigen (e.g., bovine serum albumin, casein, etc.). The amount with which the affinity is lower is dependent on the KD of the antibody, so that when the KD of the antibody is very low (that is, the antibody is highly specific), then the amount with which the affinity for the antigen is lower than the affinity for a non-specific antigen may be at least 10,000 fold. The term “kd” (sec −1 or 1/s), as used herein, refers to the dissociation rate constant of a particular antibody-antigen interaction. The value is also referred to as the koff value. The term “ka” (M-1×sec-1 or 1/M), as used herein, refers to the association rate constant of a particular antibody-antigen interaction. The term “KD” (M), as used herein, refers to the dissociation equilibrium constant of a particular antibody-antigen interaction and is obtained by dividing the kd by the ka. The term “KA” (M-1 or 1/M), as used herein, refers to the association equilibrium constant of a particular antibody-antigen interaction and is obtained by dividing the ka by the kd.

In an embodiment, the MuSK antibody-based molecules described herein have a pH-dependent binding affinity for MuSK that allows for antibody recycling to enhance antigen binding. For example, in an embodiment, the association rate constant or dissociation rate constant may differ under acidic vs. neutral vs. basic pH conditions. In one embodiment, the MuSK antibody-based molecules described herein have a higher dissociation rate constant under acidic pH conditions, e.g., pH of <7.0, compared to neutral pH conditions, e.g., pH of ˜7.0-7.9. In some embodiments, the MuSK antibody-based molecules described herein have a 2-fold to 3-fold higher dissociation rate constant (i.e., decreased binding affinity) at an acidic pH (e.g., pH ˜5.5) as compared to a neutral pH. (pH ˜7.4). In an embodiment, the MuSK antibody-based molecules bind the MuSK Fz-like domain with a higher affinity at neutral pH conditions than at acidic pH conditions. In other words, in an embodiment, the MuSK antibody-based molecules binds the MuSK Fz-like domain with a higher dissociation rate at acidic pH conditions than under neutral pH conditions. Neutral pH conditions may be defined as being a pH comprised from 7.0 to 7.9. Acidic pH conditions may be defined as being a pH being less than 7.0. Higher may mean at least 10%, 20%, 30%, 40%, 50%, 60%, 70%, 80%, 90%, 100%, 150%, 200%, 250%, 300% higher. Antibodies having this pH dependent dissociation characteristic dissociate from the antigen after binding and activation but before lysosomal degradation. Once dissociated, the antibody is transported via the neonatal Fc receptor back into circulation and is released to bind more antigen.

In some embodiments, binding of the MuSK antibodies of the present invention to their respective epitopes within the Fz-like domain activates MuSK signaling. In particular, when the MuSK antibodies of the present invention bind their respective epitope of the MuSK Fz-like domain, this binding induces MuSK phosphorylation and activation. The MuSK antibodies of the present invention induce MuSK phosphorylation by about 50% to about 100% relative to MuSK phosphorylation induced by agrin activation (as measured, e.g., in a C2C12 phosphorylation assay). In an embodiment, the MuSK antibodies of the present invention induce about 55%, 60%, 65%, 70%, 75%, 80%, 85%, 90%, 95% or 100% 95% MuSK phosphorylation (relative to MuSK phosphorylation induced by agrin activation). In an embodiment, the MuSK antibody-based molecules of the present invention induce about 90% to about 100% MuSK phosphorylation (relative to MuSK phosphorylation induced by agrin activation), upon MuSK binding. Phosphorylation of MuSK may be assessed using techniques known to the skilled person such as western blotting or a C2C12 myotube phosphorylation assay. Such antibodies activating MuSK signalling (i.e. induction of the dimerization of MuSK, induction of the tyrosine phosphorylation of MuSK) are agonist antibodies.

In some embodiments, the MuSK antibodies of the present invention, i.e., MuSK antibodies that bind to the Fz-domain of MuSK, do not interfere (i.e., do not block, impede, inhibit, or reduce) with natural ligand binding and stimulation of MuSK. In some embodiments, the MuSK antibodies co-stimulate MuSK activation with its natural ligand, i.e., agrin, to produce an additive effect of activation, e.g, MuSK phosphorylation. Thus, in some embodiments, the MuSK antibodies of the present invention potentiate natural MuSK activation, i.e., phosphorylation, induced by natural ligand binding. Such MuSK antibodies are agonist antibodies. In some embodiments, the antibodies of the invention, in combination with the natural ligand, activate MuSK (i.e., MuSK phosphorylation) to >100% of endogenous activation levels such as at least 110%, 130%, 150%, 200% of endogenous activation levels.

Accordingly, in an embodiment, activities of the MuSK antibody-based molecules of the invention include: (i) binding to an epitope of human muscle-specific tyrosine-protein kinase (MuSK), said epitope present in the MuSK Frizzled (Fz)-like domain sequence of SEQ ID NO: 130, wherein said antibody-based molecule induces MuSK phosphorylation upon binding to its epitope, and/or (ii) binding to the MuSK Fz-like domain does not block, impede, or inhibit natural or endogenous MuSK ligand induced phosphorylation, and may potentiate said natural or endogenous MuSK ligand induced phosphorylation, and (iii) binding to the MuSK Fz-like domain occurs with a higher affinity at neutral pH conditions than at acidic pH conditions.

All these features have been further defined herein.

Antibody-based molecules include, without limitation antibodies, full antibodies, epitope binding fragments of whole antibodies, antigen binding fragment of whole antibodies and antibody derivatives. An epitope binding fragment of an antibody can be obtained through the actual fragmenting of a parental antibody (for example, a Fab or (Fab)2 fragment). Alternatively, the epitope binding fragment is an amino acid sequence that comprises a portion of the amino acid sequence of such parental antibody. As used herein, a molecule is said to be a “derivative” of an antibody (or relevant portion thereof) if it is obtained through the actual chemical modification of a parent antibody or portion thereof, or if it comprises an amino acid sequence that is substantially similar to the amino acid sequence of such parental antibody or relevant portion thereof (for example, differing by less than 30%, less than 20%, less than 10%, or less than 5% from such parental molecule or such relevant portion thereof, or by 10 amino acid residues, or by fewer than 10, 9, 8, 7, 6, 5, 4, 3 or 2 amino acid residues from such parental molecule or relevant portion thereof).

In an embodiment, an antibody-based molecule of the present invention is an intact immunoglobulin or a molecule having an epitope-binding 333 acids encoding such fragments in recombinant cells (see e.g., Evans et al. “Rapid Expression Of An Anti-Human C5 Chimeric Fab Utilizing A Vector That Replicates In COS And 293 Cells,” J. Immunol. Meth. 184:123-38 (1995), which is hereby incorporated by reference in its entirety). For example, a chimeric gene encoding a portion of a F(ab′)2 fragment could include DNA sequences encoding the CH1 domain and hinge region of the heavy chain, followed by a translational stop codon to yield such a truncated antibody fragment molecule. Suitable fragments capable of binding to a desired epitope may be readily screened for utility in the same manner as an intact antibody.

Antibody derivatives include those molecules that contain at least one epitope-binding domain of an antibody, and are typically formed using recombinant techniques. One exemplary antibody derivative includes a single chain Fv (scFv). A scFv is formed from the two domains of the Fv fragment, the VL and the VH, which may be encoded by separate genes. Such gene sequences or their encoding cDNA are joined, using recombinant methods, by a flexible linker (typically of about 10, 12, 15 or more amino acid residues) that enables them to be made as a single protein chain in which the VL and VH associate to form monovalent epitope-binding molecules (see e.g., Bird et al. “Single-Chain Antigen-Binding Proteins,” Science 242:423-426 (1988); and Huston et al. “Protein Engineering Of Antibody Binding Sites: Recovery Of Specific Activity In An Anti-Digoxin Single-Chain Fv Analogue Produced In Escherichia coli,” Proc. Natl. Acad. Sci. (U.S.A.) 85:5879-5883 (1988), which are hereby incorporated by reference in their entirety). Alternatively, by employing a flexible linker that is not too short (e.g., not less than about 9 residues) to enable the VL and VH of a different single polypeptide chains to associate together, one can form a bispecific antibody, having binding specificity for two different epitopes.

In another embodiment, the antibody derivative is a divalent or bivalent single-chain variable fragment, engineered by linking two scFvs together either in tandem (i.e., tandem scFv), or such that they dimerize to form a diabody (Holliger et al. “‘Diabodies’: Small Bivalent And Bispecific Antibody Fragments,” Proc. Natl. Acad. Sci. (U.S.A.) 90(14), 6444-8 (1993), which is hereby incorporated by reference in its entirety). In yet another embodiment, the antibody is a triabody, i.e., a trivalent single chain variable fragment, engineered by linking three scFvs together, either in tandem or in a trimer formation to form a triabody. In another embodiment, the antibody is a tetrabody of four single chain variable fragments. In another embodiment, the antibody is a “linear antibody” which is an antibody comprising a pair of tandem Fd segments (VH-CH1-VH—CH1) that form a pair of antigen binding regions (see Zapata et al. Protein Eng. 8(10):1057-1062 (1995), which is hereby incorporated by reference in its entirety). In another embodiment, the antibody derivative is a minibody, consisting of the single-chain Fv regions coupled to the CH3 region (i.e., scFv-CH3).

These and other useful antibody fragments and derivatives in the context of the present invention are discussed further herein. It also should be understood that the term antibody-based molecule, unless specified otherwise, also includes antibody-like polypeptides, such as chimeric antibodies and humanized antibodies, antigen binding fragments and antibody fragments retaining the ability to specifically bind to the antigen (epitope-binding fragments, antigen binding fragments or functional fragments) provided by any known technique, such as enzymatic cleavage, peptide synthesis, and recombinant techniques.

An antibody as generated herein may be of any isotype. As used herein, “isotype” refers to the immunoglobulin class (for instance IgG1, IgG2, IgG3, IgG4, IgD, IgA, IgE, or IgM) that is encoded by heavy chain constant region genes. The choice of isotype typically will be guided by the desired effector functions, such as antibody-dependent cellular cytotoxicity (ADCC) induction. Exemplary isotypes are IgG1, IgG2, IgG3, and IgG4. Particularly useful isotypes of the MuSK antibodies disclosed herein include IgG1 and IgG2.

Either of the human light chain constant regions, kappa or lambda, may be used. If desired, the class of a MuSK antibody of the present invention may be switched by known methods. For example, an antibody of the present invention that was originally IgM may be class switched to an IgG antibody of the present invention. Further, class switching techniques may be used to convert one IgG subclass to another, for instance from IgG1 to IgG2. Thus, the effector function of the antibodies of the present invention may be changed by isotype switching to, e.g., an IgG1, IgG2, IgG3, IgG4, IgD, IgA, IgE, or IgM antibody for various therapeutic uses.

In an embodiment, one, two, or more amino acid substitutions are introduced into an IgG constant region Fc region to alter the effector function(s) of the antibody-based molecule. For example, one or more amino acids selected from amino acid residues 234, 235, 236, 237, 238, 239, 243, 265, 267, 268, 292, 297, 300, 318, 320, 322, 327, 328, 329, 330, 331, 332, and 396, numbered according to the EU numbering system (https://www.imgt.org/IMGTScientificChart/Numbering/Hu_IGHGnber.html#notes, and Edelman, G. M. et al., Proc. Natl. Acad. USA, 63, 78-85 (1969). PMID: 5257969), can be replaced with a different amino acid residue such that the antibody-based molecule has an altered affinity for an effector ligand but retains the antigen-binding ability. In an embodiment, the amino acid 234 or 235 has been replaced. In another embodiment, the amino acids 234 and 235 have been replaced. In this context, a preferred amino acid sequence of a human IgG constant Fc region comprises SEQ ID NO:266 or 267. In this context, for example, the amino acids 234 and 235 numbered according to the EU numbering system correspond to amino acids 7 and 8 in SEQ ID NO:266 and 267 (i.e. a human IgG constant Fc region of an antibody-based molecule disclosed herein), or the amino acids 234 and 235 numbered according the EU numbering system correspond to amino acids 238 and 239 in SEQ ID NO:268 and 270 (i.e. a human full length heavy chain of an antibody-based molecule disclosed herein). The positions typically differ, because variable regions vary in length, which introduces a “delta” between the numberings. In the case depicted above, that delta is 4. Accordingly, the same holds for other amino acid positions identified above (i.e. 236, 237, 238, 239, 243, 265, 267, 268, 292, 297, 300, 318, 320, 322, 327, 328, 329, 330, 331, 332, and 396) numbered according to the EU numbering system when identifying the corresponding positions in SEQ ID NO: 266 or 267 or 268 or 270. Within the application as filed, one can either refer to the position of an amino acid using the EU numbering system or using the actual position in a given Fc region (for example SEQ ID NO: 266 or 267) or in a full length heavy chain (for example SEQ ID NO: 268 or 270).

Accordingly, in an embodiment, 1, 2, 3, 4, 5, 6, 7, 8, 9, 10, 11, 12, 13, 14, 15, 16 or 17 amino acid substitutions are introduced into SEQ ID NO: 266 or 267. In an embodiment, 1, 2, 3, 4 amino acid substitutions are introduced into SEQ ID NO:266 or 267. In an embodiment, 1 or 2 amino acid substitutions are introduced into SEQ ID NO:266 or 267. Accordingly, in an embodiment, 1, 2, 3, 4, 5, 6, 7, 8, 9, 10, 11, 12, 13, 14, 15, 16 or 17 amino acid substitutions are introduced into SEQ ID NO: 266 or 267 and said substitutions are introduced at amino acid positions selected from amino acid residues 234, 235, 236, 237, 239, 243, 267, 292, 297, 300, 318, 320, 322, 328, 330, 332, and 396 numbered according the EU numbering system of said sequence. In an embodiment, 1 or 2 amino acid substitutions are introduced into SEQ ID NO:266 or 267. In an embodiment, the amino acid 234 or 235 numbered according to the EU numbering system of SEQ ID NO: 266 or 267 has been replaced. In another embodiment, the amino acids 234 and 235 numbered according to the EU numbering system of SEQ ID NO: 266 or 267 have been replaced.

The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In an embodiment, one or more amino acid substitutions may be introduced into the Fc region of the antibody-based molecule described herein to remove potential glycosylation sites on the Fc region, which may reduce Fc receptor binding (see, e.g., Shields R L et al., (2001) J Biol Chem 276: 6591-604, which is herein incorporated by reference in its entirety). In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

In a first embodiment, one or more of the following mutations have been introduced into the constant region of the antibody-based molecule described herein (all numbered according to the EU numbering system): an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In a second embodiment, one or more of the following mutations have been introduced into the constant region of the antibody-based molecule described herein (all numbered according to the EU numbering system): an L234A and/or an L235A substitution; an L234A and an L235A substitution; an L234A, an L235A and a P329G substitution; an L234A, an L235A and a G236K substitution; an L234A, an L235A and a G236E substitution; an L234A, an L235A and a G236R substitution; an L234A and a G236R substitution; an L234A, L235S and a G236R substitution; an L234A, L235T and a G236R substitution; an L234D, L235H and a G236R substitution; an L234D, L235K and a G236R substitution; an L234D and a G236R substitution; an L234D, L235Q and a G236R substitution; an L234D, L235S and a G236R substitution; an L234E, L235D and a G236R substitution; an L234E, L235H and a G236R substitution; an L234E, L2351 and a G236R substitution; an L234G, L235H and a G236R substitution; an L234G, L235Q and a G236R substitution; an L234G, L235S and a G236R substitution; an L234H, L2351 and a G236R substitution; an L234H, L235S and a G236R substitution; an L234K, L235Q and a G236R substitution; an L234K, L235R and a G236R substitution; an L234K, L235S and a G236R substitution; an L234K, L235T and a G236R substitution; an L234K, L235V and a G236R substitution; an L234Q, L235A and a G236R substitution; an L234Q, L235D and a G236R substitution; an L234Q, L235H and a G236R substitution; an L234Q and a G236R substitution; an L234Q, L235Q and a G236R substitution; an L234Q, L235R and a G236R substitution; an L234Q, L235S and a G236R substitution; an L234Q, L235T and a G236R substitution; an L234Q, L235V and a G236R substitution; an L234R, L235D and a G236R substitution; an L234R, L235E and a G236R substitution; an L234R, L235H and a G236R substitution; an L234R, L2351 and a G236R substitution; an L234R, L235K and a G236R substitution; an L234R and a G236R substitution; an L234R, L235Q and a G236R substitution; an L234R, L235R and a G236R substitution; an L234R, L235T and a G236R substitution; an L234S, L235E and a G236R substitution; an L234S, L235G and a G236R substitution; an L234S, L235H and a G236R substitution; an L234S, L2351 and a G236R substitution; an L234S and a G236R substitution; an L234S, L235R and a G236R substitution; L234S, L235T and a G236R substitution; L234S, L235V and a G236R substitution; an L234T, L235A and a G236R substitution; an L234T, L235D and a G236R, an L234T, L235H and a G236R substitution; an L234T, L2351 and a G236R substitution; an L234T, L235K and a G236R substitution; an L234T, L235Q and a G236R substitution; an L234T, L235R and a G236R substitution; an L234T, L235S and a G236R substitution; an L234T, L235T and a G236R substitution; an L234T, L235V and a G236R substitution; a G236R and an L328R substitution; an L234A, an L235A, a G237A, a P238S, an H268A, an A330S and a P331S substitution; an E233P, an L234V, an L235A, a G326 deletion, an A327G, an A330S and a P331S substitution; an L235A and a G236R substitution; an L235S and a G236R substitution.

In a third embodiment, one or more of the following mutations have been introduced into the Fc region SEQ ID NO: 266 or SEQ ID NO: 267 of the antibody-based molecule described herein (all numbered according to the EU numbering system): an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In a fourth embodiment, one or more of the following mutations have been introduced into the Fc region SEQ ID NO: 266 or SEQ ID NO: 267 of the antibody-based molecule described herein (all numbered according to the EU numbering system): an L234A and/or an L235A substitution; an L234A and an L235A substitution; an L234A, an L235A and a P329G substitution; an L234A, an L235A and a G236K substitution; an L234A, an L235A and a G236E substitution; an L234A, an L235A and a G236R substitution; an L234A and a G236R substitution; an L234A, L235S and a G236R substitution; an L234A, L235T and a G236R substitution; an L234D, L235H and a G236R substitution; an L234D, L235K and a G236R substitution; an L234D and a G236R substitution; an L234D, L235Q and a G236R substitution; an L234D, L235S and a G236R substitution; an L234E, L235D and a G236R substitution; an L234E, L235H and a G236R substitution; an L234E, L2351 and a G236R substitution; an L234G, L235H and a G236R substitution; an L234G, L235Q and a G236R substitution; an L234G, L235S and a G236R substitution; an L234H, L2351 and a G236R substitution; an L234H, L235S and a G236R substitution; an L234K, L235Q and a G236R substitution; an L234K, L235R and a G236R substitution; an L234K, L235S and a G236R substitution; an L234K, L235T and a G236R substitution; an L234K, L235V and a G236R substitution; an L234Q, L235A and a G236R substitution; an L234Q, L235D and a G236R substitution; an L234Q, L235H and a G236R substitution; an L234Q and a G236R substitution; an L234Q, L235Q and a G236R substitution; an L234Q, L235R and a G236R substitution; an L234Q, L235S and a G236R substitution; an L234Q, L235T and a G236R substitution; an L234Q, L235V and a G236R substitution; an L234R, L235D and a G236R substitution; an L234R, L235E and a G236R substitution; an L234R, L235H and a G236R substitution; an L234R, L2351 and a G236R substitution; an L234R, L235K and a G236R substitution; an L234R and a G236R substitution; an L234R, L235Q and a G236R substitution; an L234R, L235R and a G236R substitution; an L234R, L235T and a G236R substitution; an L234S, L235E and a G236R substitution; an L234S, L235G and a G236R substitution; an L234S, L235H and a G236R substitution; an L234S, L2351 and a G236R substitution; an L234S and a G236R substitution; an L234S, L235R and a G236R substitution; L234S, L235T and a G236R substitution; L234S, L235V and a G236R substitution; an L234T, L235A and a G236R substitution; an L234T, L235D and a G236R, an L234T, L235H and a G236R substitution; an L234T, L2351 and a G236R substitution; an L234T, L235K and a G236R substitution; an L234T, L235Q and a G236R substitution; an L234T, L235R and a G236R substitution; an L234T, L235S and a G236R substitution; an L234T, L235T and a G236R substitution; an L234T, L235V and a G236R substitution; a G236R and an L328R substitution; an L234A, an L235A, a G237A, a P238S, an H268A, an A330S and a P331S substitution; an E233P, an L234V, an L235A, a G326 deletion, an A327G, an A330S and a P331S substitution; an L235A and a G236R substitution; an L235S and a G236R substitution.

In an embodiment, one or more of the following mutations are introduced into the Fc region SEQ ID NO: 266 or SEQ ID NO: 267 of the antibody-based molecule described herein: an L234A and/or an L235A substitution (numbered according to the EU numbering system). In an embodiment, the following mutations are introduced into the Fc region SEQ ID NO: 266 or SEQ ID NO: 267 of the antibody-based molecule described herein: an L234A and an L235A substitutions numbered according to the EU numbering system. This embodiment results in an antibody-based molecule with a heavy chain represented by SEQ ID NO:268 or 270.

Such an antibody with altered, diminished even abolished effector function is attractive in the context of the invention.

In an embodiment, an anti-MuSK antibody or antigen binding fragment thereof is provided which:
is an agonist MuSK antibody and/or
has reduced or eliminated effector function.

This antibody or antigen-binding fragment thereof is preferably for use in the treatment of a neuromuscular disorder in a human subject.

In an embodiment, an anti-MuSK antibody or antigen binding fragment thereof is provided which:
binds the MuSK Frizzled (Fz)-like domain sequence of SEQ ID NO: 129,
is an agonist MuSK antibody and
has reduced or eliminated effector function.

This antibody or antigen-binding fragment thereof is preferably for use in the treatment of a neuromuscular disorder in a human subject.

Reduced or eliminated effector function may be obtained as earlier described herein by introducing mutation in the human IgG constant Fc region. Preferably, at least 1, 2, 3, 4, 5, 6, 7, 8, 9, 10, 11, 12, 13, 14, 15, 16 or 17 amino acid substitutions are introduced into said Fc region. Preferably, at least 1, 2, 3, 4, amino acid substitutions are introduced into said Fc region.

Said Fc region may comprise SEQ ID NO: 266 or 267 and said substitutions are introduced at amino acid positions selected from amino acid residues 234, 235, 236, 237, 238, 239, 243, 265, 267, 268, 292, 297, 300, 318, 320, 322, 327, 328, 329, 330, 331, 332, and 396 numbered according to the EU numbering system of said sequence.

In an embodiment, said Fc region may comprise SEQ ID NO: 266 or 267 and said substitutions are introduced at amino acid positions selected from amino acid residues 234 or 235 of said sequence numbered according to the EU numbering system.

In an embodiment, said Fc region may comprise SEQ ID NO: 266 or 267 and said substitutions are introduced at amino acid positions selected from amino acid residues 234 and 235 numbered according to the EU numbering system of said sequence.

In an embodiment, one or more of the following mutations (all numbered according to the EU numbering system) are introduced into the human IgG constant Fc region SEQ ID NO: 266 or SEQ ID NO: 267 of the antibody-based molecule described herein: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In one embodiment, each of the combinations of mutations described earlier in the fourth embodiment of this application in the human IgG constant Fc region of the antibody-based molecule described herein may be made.

In a preferred embodiment, L234A or L235A substitution is introduced into the human IgG constant Fc region of the antibody-based molecule described herein. In a more preferred embodiment, L234A and L235A substitutions are introduced into the human IgG constant Fc region of the antibody-based molecule described herein. This embodiment results in an antibody-based molecule with a heavy chain represented by SEQ ID NO:268 or 270.

In an even more preferred embodiment, said anti-MuSK antibody or antigen binding fragment thereof, comprises:
a) a heavy chain variable domain (VH) comprising an amino acid sequence that is at least 80% identical or similar to SEQ ID NO: 234 and
b) a light chain variable domain (VL) comprising an amino acid sequence that is at least 80% identical or similar to SEQ ID NO: 235.

Within this context, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

Preferred anti-MuSK antibody or antigen binding fragment thereof, comprises:
a) A full length heavy chain comprising SEQ ID NO: 268 and
b) A full length light chain comprising SEQ ID NO: 269.

Preferred anti-MuSK antibody or antigen binding fragment thereof, comprises:
c) A full length heavy chain comprising SEQ ID NO: 270 and
d) A full length light chain comprising SEQ ID NO: 271.

In an embodiment, the antibody-based molecules of the present invention are “humanized,” particularly if they are to be employed for therapeutic purposes. The term “humanized” refers to a chimeric molecule, generally prepared using recombinant techniques, having an antigen-binding site derived from an immunoglobulin from a non-human species and a remaining immunoglobulin structure based upon the structure and/or sequence of a human immunoglobulin. The antigen-binding site may comprise either complete non-human antibody variable domains fused to human constant domains, or only the complementarity determining regions (CDRs) of such variable domains grafted to appropriate human framework regions of human variable domains. The framework residues of such humanized molecules may be wild-type (e.g., fully human) or they may be modified to contain one or more amino acid substitutions not found in the human antibody whose sequence has served as the basis for humanization. Humanization lessens or eliminates the likelihood that a constant region of the molecule will act as an immunogen in human individuals, but the possibility of an immune response to the foreign variable region remains (LoBuglio, A.F. et al. “Mouse/Human Chimeric Monoclonal Antibody In Man: Kinetics And Immune Response,” Proc. Natl. Acad. Sci. USA 86:4220-4224 (1989), which is hereby incorporated by reference in its entirety). Another approach focuses not only on providing human-derived constant regions, but modifying the variable regions so as to reshape them as closely as possible to human form. The variable regions of both heavy and light chains contain three complementarity-determining regions (CDRs) which vary in response to the antigens in question and determine binding capability. The CDRs are flanked by four framework regions (FRs) which are relatively conserved in a given species and which putatively provide a scaffolding for the CDRs. When non-human antibodies are prepared with respect to a particular antigen, the variable regions can be “reshaped” or “humanized” by grafting CDRs derived from non-human antibody onto the FRs present in the human antibody to be modified. Suitable methods for humanizing the non-human antibody described herein are known in the art see e.g., Sato, K. et al., Cancer Res 53:851-856 (1993); Riechmann, L. et al., “Reshaping Human Antibodies for Therapy,” Nature 332:323-327 (1988); Verhoeyen, M. et al., “Reshaping Human Antibodies: Grafting An Antilysozyme Activity,” Science 239:1534-1536 (1988); Kettleborough, C. A. et al., “Humanization Of A Mouse Monoclonal Antibody By CDR-Grafting: The Importance Of Framework Residues On Loop Conformation,” Protein Engineering 4:773-3783 (1991); Maeda, H. et al., “Construction Of Reshaped Human Antibodies With HIV-Neutralizing Activity,” Human Antibodies Hybridoma 2:124-134 (1991); Gorman, S. D. et al., “Reshaping A Therapeutic CD4 Antibody,” Proc. Natl. Acad. Sci. USA 88:4181-4185 (1991); Tempest, P. R. et al., “Reshaping A Human Monoclonal Antibody To Inhibit Human Respiratory Syncytial Virus Infection In Vivo,” Bio/Technology 9:266-271 (1991); Co, M. S. et al., “Humanized Antibodies For Antiviral Therapy,” Proc. Natl. Acad. Sci. USA 88:2869-2873 (1991); Carter, P. et al., “Humanization Of An Anti-p185her2 Antibody For Human Cancer Therapy,” Proc. Natl. Acad. Sci. USA 89:4285-4289 (1992); and Co, M.S. et al., “Chimeric And Humanized Antibodies With Specificity For The CD33 Antigen,” J. Immunol. 148:1149-1154 (1992), which are hereby incorporated by reference in their entirety. In some embodiments, humanized MuSK antibodies of the present invention preserve all CDR sequences (for example, a humanized antibody containing all six CDRs from the llama or mouse antibody). In other embodiments, humanized MuSK antibodies of the present invention have one or more CDRs (one, two, three, four, five, six) which are altered with respect to the original antibody. Methods of humanizing an antibody are well-known in the art and suitable for humanizing the antibodies disclosed herein (see, e.g., U.S. Pat. No. 5,225,539 to Winter; U.S. Pat. Nos. 5,530,101 and 5,585,089 to Queen and Selick; U.S. Pat. No. 5,859,205 to Robert et al.; U.S. Pat. No. 6,407,213 to Carter; and U.S. Pat. No. 6,881,557 to Foote, which are hereby incorporated by reference in their entirety).

In some antibodies only part of a CDR, namely the subset of CDR residues required for binding termed the “specificity determining residues” (“SDRs”), are needed to retain binding of the antibody. CDR residues not contacting antigen and not in the SDRs can be identified based on previous studies from regions of Kabat CDRs lying outside Chothia hypervariable loops (see, Kabat et al., SEQUENCES OF PROTEINS OF IMMUNOLOGICAL INTEREST, National Institutes of Health Publication No. 91-3242 (1992); Chothia, C. et al., “Canonical Structures For The Hypervariable Regions Of Immunoglobulins,” J. Mol. Biol. 196:901-917 (1987), which are hereby incorporated by reference in their entirety), by molecular modeling and/or empirically, or as described in Gonzales, N. R. et al., “SDR Grafting Of A Murine Antibody Using Multiple Human Germline Templates To Minimize Its Immunogenicity,” Mol. Immunol. 41:863-872 (2004), which is hereby incorporated by reference in its entirety. In such humanized antibodies, at positions in which one or more donor CDR residues is absent or in which an entire donor CDR is omitted, the amino acid residue occupying the position can be an amino acid residue occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of non-human amino acids in a humanized antibody and consequently decreasing potential immunogenicity. However, substitutions can also cause changes of affinity, and significant reductions in affinity are preferably avoided. Substitutions may also cause changes of activity. Such substitutions causing a significant reduction in activity are also preferably avoided. In this context, the antibody or antibody fragment should still exhibit a detectable activity of the antibody as earlier defined herein or an activity of the antibody at least to some extent. Positions for substitution within CDRs and amino acids to substitute can also be selected empirically.

Phage display technology can alternatively be used to increase (or decrease) CDR affinity of the antibody-based molecules of the present invention. This technology, referred to as affinity maturation, employs mutagenesis or “CDR walking” and re-selection using the target antigen or an antigenic fragment thereof to identify antibodies having CDRs that bind with higher (or lower) affinity to the antigen when compared with the initial or parental antibody (see, e.g. Glaser et al., “Antibody Engineering By Codon-Based Mutagenesis In A Filamentous Phage Vector System,” J. Immunology 149:3903-3913 (1992), which is hereby incorporated by reference in its entirety). Mutagenizing entire codons rather than single nucleotides results in a semi-randomized repertoire of amino acid mutations. Libraries can be constructed consisting of a pool of variant clones each of which differs by a single amino acid alteration in a single CDR from another member of such library and which contain variants potentially representing each possible amino acid substitution for each CDR residue. Mutants with increased (or decreased) binding affinity for the antigen can be screened by contacting the immobilized mutants with labeled antigen. Any screening method known in the art can be used to identify variant antibody-based binding molecules with increased or decreased affinity to the antigen (e.g., ELISA) (See Wu, H. et al., “Stepwise In Vitro Affinity Maturation Of Vitaxin, An Alphav Beta3-Specific Humanized mAb,” Proc. Natl. Acad. Sci. USA 95:6037-6042 (1998); Yelton et al., “Affinity Maturation Of The BR96 Anti-Carcinoma Antibody By Codon-Based Mutagenesis,” J. Immunology 155:1994 (1995), which are hereby incorporated by reference in their entirety). CDR walking, which randomizes the light chain may be used (see, Schier, R. et al., “Isolation Of Picomolar Affinity Anti-c-erbB-2 Single-Chain Fv By Molecular Evolution Of The Complementarity Determining Regions In The Center Of The Antibody Binding Site,” J. Mol. Biol. 263:551-567 (1996), which is hereby incorporated by reference in its entirety).

Methods for affinity maturation of the MuSK antibody molecule are described herein and disclosed for example, in Krause, J. C. et al., “An Insertion Mutation That Distorts Antibody Binding Site Architecture Enhances Function of a Human Antibody,” MBio. 2(1): e00345-10 (2011); Kuan, C.T. et al., “Affinity-Matured Anti-Glycoprotein NMB Recombinant Immunotoxins Targeting Malignant Gliomas And Melanomas,” Int. J. Cancer 10.1002/ijc.25645 (2010); Hackel, B. J. et al., “Stability And CDR Composition Biases Enrich Binder Functionality Landscapes,” J. Mol. Biol. 401(1):84-96 (2010); Montgomery, D. L. et al., “Affinity Maturation And Characterization Of A Human Monoclonal Antibody Against HIV-1 gp41,” MAbs 1(5):462-474 (2009); Gustchina, E. et al., “Affinity Maturation By Targeted Diversification Of The CDR-H2 Loop Of A Monoclonal Fab Derived From A Synthetic Naïve Human Antibody Library And Directed Against The Internal Trimeric Coiled-Coil Of Gp41 Yields A Set Of Fabs With Improved HIV-1 Neutralization Potency And Breadth,” Virology 393(1):112-119 (2009); Finlay, W. J. et al., “Affinity Maturation Of A Humanized Rat Antibody For Anti-RAGE Therapy: Comprehensive Mutagenesis Reveals A High Level Of Mutational Plasticity Both Inside And Outside The Complementarity-Determining Regions,” J. Mol. Biol. 388(3):541-558 (2009); Bostrom, J. et al., “Improving Antibody Binding Affinity And Specificity For Therapeutic Development,” Methods Mol. Biol. 525:353-376 (2009); Steidl, S. et al., “In Vitro Affinity Maturation Of Human GM-CSF Antibodies By Targeted CDR-Diversification,” Mol. Immunol. 46(1):135-144 (2008); and Barderas, R. et al., “Affinity Maturation Of Antibodies Assisted By In Silico Modeling,” Proc. Natl. Acad. Sci. USA 105(26):9029-9034 (2008), which are hereby incorporated by reference in their entirety.

In the context of this application, an amino acid alteration (change or modification) may be an amino acid substitution, addition, deletion or chemical modification.

In an embodiment, the MuSK-antibody based molecule as described herein comprises the amino acid sequence of any one, any two, any three, any four, any five, or any six CDRs as provided in Tables 1 and 2 herein.

In one embodiment, the antibody-based molecule that binds to human muscle-specific tyrosine-protein kinase (MuSK) comprises a heavy chain variable domain, where the heavy chain variable domain comprises:
(i) a complementarity-determining region 1 (CDR-H1) comprising an amino acid sequence of any one of SEQ ID NOs: 1-16, 135, 136, 147-149 or a modified amino acid sequence of any one of SEQ ID NOs: 1-16, 135, 136, or 147-149 said modified sequence having 1, 2, 3, 4 or 5 amino acid alterations relative to SEQ ID NOs: 1-16, 135, 136 or 147-149;
(ii) a complementarity-determining region 2 (CDR-H2) comprising an amino acid sequence of any one of SEQ ID NOs: 17-32, 137, 138, 150-155 or a modified amino acid sequence of any one of SEQ ID NOs: 17-32, 137, 138, or 150-155 said modified sequences having 1, 2, 3, 4 or 5 amino acid alterations relative to SEQ ID NOs: 17-32, 137, 138, or 150-155; and
(iii) a complementarity-determining region 3 (CDR-H3) comprising an amino acid sequence of any one of SEQ ID NOs: 33-48, 139, 140, 156-158, 240-251, or a modified amino acid sequence of any one of SEQ ID NO: 33-48, 139, 140, 156-158, or 240-251, said modified sequence having 1, 2, 3, 4 or 5 amino acid alterations relative to SEQ ID NOs: 33-48, 139, 140, 156-158, or 240-251.

In an embodiment, the antibody-based molecule that binds to human muscle-specific tyrosine-protein kinase (MuSK) comprises: (i) a heavy chain variable domain comprising the CDR-H1 of SEQ ID NO: 1 or having 1, 2, 3, 4 or 5 amino acid alterations relative to SEQ ID NO: 1, the CDR-H2 of SEQ ID NO: 17 or having 1, 2, 3, 4 or 5 amino acid alterations relative to SEQ ID NO:17, and the CDR-H3 of SEQ ID NO: 33 or having 1, 2, 3, 4 or 5 amino acid alterations relative to SEQ ID NO:33; (ii) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 34 or having 1, 2, 3, 4 or 5 amino acid alterations relative to SEQ ID NO:34; (iii) a heavy chain variable domain comprising the CDR-H1 of SEQ ID NO: 3 or having 1, 2, 3, 4 or 5 amino acid alterations relative to SEQ ID NO:3, the CDR-H2 of SEQ ID NO: 19 or having 1, 2, 3, 4 or 5 amino acid alterations relative to SEQ ID NO: 19, and the CDR-H3 of SEQ ID NO: 35 or having 1, 2, 3, 4 or 5 amino acid alterations relative to SEQ ID NO:35; (iv) a heavy chain variable domain comprising the CDR-H1 of SEQ ID NO: 4 or having 1, 2, 3, 4 or 5 amino acid alterations relative to SEQ ID NO:4, the CDR-H2 of SEQ ID NO: 20 or having 1, 2, 3, 4 or 5 amino acid alterations relative to SEQ ID NO:20, and the CDR-H3 of SEQ ID NO: 36 or having 1, 2, 3, 4 or 5 amino acid alterations relative to SEQ ID NO:36; (v) a heavy chain variable domain comprising the CDR-H1 of SEQ ID NO: 5 or having 1, 2, 3, 4 or 5 amino acid alterations relative to SEQ ID NO:5, the CDR-H2 of SEQ ID NO: 21 or having 1, 2, 3, 4 or 5 amino acid alterations relative to SEQ ID NO:21, and the CDR-H3 of SEQ ID NO: 37 or having 1, 2, 3, 4 or 5 amino acid alterations relative to SEQ ID NO:37; (vi) a heavy chain variable domain comprising the CDR-H1 of SEQ ID NO: 6 or having 1, 2, 3, 4 or 5 amino acid alterations relative to SEQ ID NO:6, the CDR-H2 of SEQ ID NO: 22 or having 1, 2, 3, 4 or 5 amino acid alterations relative to SEQ ID NO:22, and the CDR-H3 of SEQ ID NO: 38 or having 1, 2, 3, 4 or 5 amino acid alterations relative to SEQ ID NO:38; (vii) a heavy chain variable domain comprising the CDR-H1 of SEQ ID NO: 7 or having 1, 2, 3, 4 or 5 amino acid alterations relative to SEQ ID NO:7, the CDR-H2 of SEQ ID NO: 23 or having 1, 2, 3, 4 or 5 amino acid alterations relative to SEQ ID NO:23, and the CDR-H3 of SEQ ID NO: 39 or having 1, 2, 3, 4 or 5 amino acid alterations relative to SEQ ID NO:39; (viii) a heavy chain variable domain comprising the CDR-H1 of SEQ ID NO: 8 or having 1, 2, 3, 4 or 5 amino acid alterations relative to SEQ ID NO:8, the CDR-H2 of SEQ ID NO: 24 or having 1, 2, 3, 4 or 5 amino acid alterations relative to SEQ ID NO:24, and the CDR-H3 of SEQ ID NO: 40 or having 1, 2, 3, 4 or 5 amino acid alterations relative to SEQ ID NO:40; (ix) a heavy chain variable domain comprising the CDR-H1 of SEQ ID NO: 9 or having 1, 2, 3, 4 or 5 amino acid alterations relative to SEQ ID NO:9, the CDR-H2 of SEQ ID NO: 25 or having 1, 2, 3, 4 or 5 amino acid alterations relative to SEQ ID NO:25, and the CDR-H3 of SEQ ID NO: 41 or having 1, 2, 3, 4 or 5 amino acid alterations relative to SEQ ID NO:41; (x) a heavy chain variable domain comprising the CDR-H1 of SEQ ID NO: 10 or having 1, 2, 3, 4 or 5 amino acid alterations relative to SEQ ID NO:10, the CDR-H2 of SEQ ID NO: 26 or having 1, 2, 3, 4 or 5 amino acid alterations relative to SEQ ID NO:26, and the CDR-H3 of SEQ ID NO: 42 or having 1, 2, 3, 4 or 5 amino acid alterations relative to SEQ ID NO:42; (xi) a heavy chain variable domain comprising the CDR-H1 of SEQ ID NO: 11 or having 1, 2, 3, 4 or 5 amino acid alterations relative to SEQ ID NO:11, the CDR-H2 of SEQ ID NO: 27 or having 1, 2, 3, 4 or 5 amino acid alterations relative to SEQ ID NO:27, and the CDR-H3 of SEQ ID NO: 43 or having 1, 2, 3, 4 or 5 amino acid alterations relative to SEQ ID NO:43; (xii) a heavy chain variable domain comprising the CDR-H1 of SEQ ID NO: 12 or having 1, 2, 3, 4 or 5 amino acid alterations relative to SEQ ID NO:12, the CDR-H2 of SEQ ID NO: 28 or having 1, 2, 3, 4 or 5 amino acid alterations relative to SEQ ID NO:28, and the CDR-H3 of SEQ ID NO: 44 or having 1, 2, 3, 4 or 5 amino acid alterations relative to SEQ ID NO:44; (xiii) a heavy chain variable domain comprising the CDR-H1 of SEQ ID NO: 13 or having 1, 2, 3, 4 or 5 amino acid alterations relative to SEQ ID NO:13, the CDR-H2 of SEQ ID NO: 29 or having 1, 2, 3, 4 or 5 amino acid alterations relative to SEQ ID NO:29, and the CDR-H3 of SEQ ID NO: 45 or having 1, 2, 3, 4 or 5 amino acid alterations relative to SEQ ID NO:45; (xiv) a heavy chain variable domain comprising the CDR-H1 of SEQ ID NO: 14 or having 1, 2, 3, 4 or 5 amino acid alterations relative to SEQ ID NO:14, the CDR-H2 of SEQ ID NO: 30 or having 1, 2, 3, 4 or 5 amino acid alterations relative to SEQ ID NO:30, and the CDR-H3 of SEQ ID NO: 46 or having 1, 2, 3, 4 or 5 amino acid alterations relative to SEQ ID NO:46; (xv) a heavy chain variable domain comprising the CDR-H1 of SEQ ID NO: 15 or having 1, 2, 3, 4 or 5 amino acid alterations relative to SEQ ID NO:15, the CDR-H2 of SEQ ID NO: 31 or having 1, 2, 3, 4 or amino acid alterations relative to SEQ ID NO:31, and the CDR-H3 of SEQ ID NO: 47 or having 1, 2, 3, 4 or 5 amino acid alterations relative to SEQ ID NO:47; (xvi) a heavy chain variable domain comprising the CDR-H1 of SEQ ID NO: 16 or having 1, 2, 3, 4 or 5 amino acid alterations relative to SEQ ID NO:16, the CDR-H2 of SEQ ID NO: 32 or having 1, 2, 3, 4 or 5 amino acid alterations relative to SEQ ID NO:32, and the CDR-H3 of SEQ ID NO: 48 or having 1, 2, 3, 4 or 5 amino acid alterations relative to SEQ ID NO:48; (xvii) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 139 or having 1, 2, 3, 4 or 5 amino acid alterations relative to SEQ ID NO:139; and (xviii) a heavy chain variable domain comprising the CDR-H1 of SEQ ID NO: 136 or having 1, 2, 3, 4 or 5 amino acid alterations relative to SEQ ID NO:136, the CDR-H2 of SEQ ID NO: 138 or having 1, 2, 3, 4 or 5 amino acid alterations relative to SEQ ID NO:138, and the CDR-H3 of SEQ ID NO: 140 or having 1, 2, 3, 4 or amino acid alterations relative to SEQ ID NO:140. The sequences of the heavy chain CDRs are provided in Table 1.

In an embodiment, the antibody-based molecule that binds to human muscle-specific tyrosine-protein kinase (MuSK) comprises: (ii.a) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 240 (X2m1) or having 1, 2, 3, 4 or 5 amino acid alterations relative to SEQ ID NO:240; (ii.b) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 241 (X2m2) or having 1, 2, 3, 4 or 5 amino acid alterations relative to SEQ ID NO:241; (ii.c) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 242 (X2m3) or having 1, 2, 3, 4 or 5 amino acid alterations relative to SEQ ID NO:242; (ii.d) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 243 (X2m4) or having 1, 2, 3, 4 or 5 amino acid alterations relative to SEQ ID NO:243; (ii.e) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 244 (X2m5) or having 1, 2, 3, 4 or 5 amino acid alterations relative to SEQ ID NO:244; (ii.f) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 245 (X2m6) or having 1, 2, 3, 4 or 5 amino acid alterations relative to SEQ ID NO:245; (ii.g) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 246 (X2m7) or having 1, 2, 3, 4 or 5 amino acid alterations relative to SEQ ID NO:246; (ii.h) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 247 (X2m8) or having 1, 2, 3, 4 or 5 amino acid alterations relative to SEQ ID NO:247.

In an embodiment, the antibody-based molecule that binds to human muscle-specific tyrosine-protein kinase (MuSK) comprises: (xvii.a) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 248 (X17m1) or having 1, 2, 3, 4 or 5 amino acid alterations relative to SEQ ID NO:248; (xvii.b) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 249(X17m2) or having 1, 2, 3, 4 or 5 amino acid alterations relative to SEQ ID NO:249; (xvii.c) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 250 (X17m3) or having 1, 2, 3, 4 or 5 amino acid alterations relative to SEQ ID NO:250; (xvii.d) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 251 (X17m6) or having 1, 2, 3, 4 or 5 amino acid alterations relative to SEQ ID NO:251.

In an embodiment, the antibody-based molecule that binds to human muscle-specific tyrosine-protein kinase (MuSK) comprises a heavy chain variable domain, where the heavy chain variable domain comprises: (xix) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 150 or having 1, 2, 3, 4 or 5 amino acid alterations relative to SEQ ID NO:150, and the CDR-H3 of SEQ ID NO: 156 or having 1, 2, 3, 4 or 5 amino acid alterations relative to SEQ ID NO:156; (xx) a heavy chain variable domain comprising the CDR-H1 of SEQ ID NO: 148 or having 1, 2, 3, 4 or 5 amino acid alterations relative to SEQ ID NO:148, the CDR-H2 of SEQ ID NO: 151 or having 1, 2, 3, 4 or 5 amino acid alterations relative to SEQ ID NO:151 and the CDR-H3 of SEQ ID NO: 157 or having 1, 2, 3, 4 or amino acid alterations relative to SEQ ID NO:157; (xxi) a heavy chain variable domain comprising the CDR-H1 of SEQ ID NO: 149 or having 1, 2, 3, 4 or 5 amino acid alterations relative to SEQ ID NO:149, the CDR-H2 of SEQ ID NO: 152 or having 1, 2, 3, 4 or 5 amino acid alterations relative to SEQ ID NO:152, and the CDR-H3 of SEQ ID NO: 158 or having 1, 2, 3, 4 or 5 amino acid alterations relative to SEQ ID NO:158.

In an embodiment, the antibody-based molecule that binds to human muscle-specific tyrosine-protein kinase (MuSK) comprises a heavy chain variable domain, where the heavy chain variable domain comprises (xxii) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 153 or having 1, 2, 3, 4 or 5 amino acid alterations relative to SEQ ID NO:153, and the CDR-H3 of SEQ ID NO:156 (3B2g1 m1/3B2g2m1) or having 1, 2, 3, 4 or 5 amino acid alterations relative to SEQ ID NO:156; (xxiii) a heavy chain variable domain comprising the CDR-H1 of SEQ ID NO: 147 or having 1, 2, 3, 4 or amino acid alterations relative to SEQ ID NO:147, the CDR-H2 of SEQ ID NO: 154 or having 1, 2, 3, 4 or 5 amino acid alterations relative to SEQ ID NO:154, and the CDR-H3 of SEQ ID NO: 156 (3B2g1m2/3B2g2m2) or having 1, 2, 3, 4 or 5 amino acid alterations relative to SEQ ID NO:156; (xxiv) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 155 or having 1, 2, 3, 4 or 5 amino acid alterations relative to SEQ ID NO:155, and the CDR-H3 of SEQ ID NO: 156 (3B2g1m4/3B2g2m4) or having 1, 2, 3, 4 or 5 amino acid alterations relative to SEQ ID NO:156. The sequences of the heavy chain CDRs are provided in Table 1.

In an embodiment, the antibody-based molecule that binds to human muscle-specific tyrosine-protein kinase (MuSK) comprises: (i) a heavy chain variable domain comprising the CDR-H1 of SEQ ID NO: 1, the CDR-H2 of SEQ ID NO: 17, and the CDR-H3 of SEQ ID NO: 33; (ii) a heavy chain variable domain comprising the CDR-H1 of SEQ ID NO: 2, the CDR-H2 of SEQ ID NO: 18, and the CDR-H3 of SEQ ID NO: 34; (iii) a heavy chain variable domain comprising the CDR-H1 of SEQ ID NO: 3, the CDR-H2 of SEQ ID NO: 19, and the CDR-H3 of SEQ ID NO: 35; (iv) a heavy chain variable domain comprising the CDR-H1 of SEQ ID NO: 4, the CDR-H2 of SEQ ID NO: 20, and the CDR-H3 of SEQ ID NO: 36; (v) a heavy chain variable domain comprising the CDR-H1 of SEQ ID NO: 5, the CDR-H2 of SEQ ID NO: 21, and the CDR-H3 of SEQ ID NO: 37; (vi) a heavy chain variable domain comprising the CDR-H1 of SEQ ID NO: 6, the CDR-H2 of SEQ ID NO: 22, and the CDR-H3 of SEQ ID NO: 38; (vii) a heavy chain variable domain comprising the CDR-H1 of SEQ ID NO: 7, the CDR-H2 of SEQ ID NO: 23, and the CDR-H3 of SEQ ID NO: 39; (viii) a heavy chain variable domain comprising the CDR-H1 of SEQ ID NO: 8, the CDR-H2 of SEQ ID NO: 24, and the CDR-H3 of SEQ ID NO: 40; (ix) a heavy chain variable domain comprising the CDR-H1 of SEQ ID NO: 9, the CDR-H2 of SEQ ID NO: 25, and the CDR-H3 of SEQ ID NO: 41; (x) a heavy chain variable domain comprising the CDR-H1 of SEQ ID NO: 10, the CDR-H2 of SEQ ID NO: 26, and the CDR-H3 of SEQ ID NO: 42; (xi) a heavy chain variable domain comprising the CDR-H1 of SEQ ID NO: 11, the CDR-H2 of SEQ ID NO: 27, and the CDR-H3 of SEQ ID NO: 43; (xii) a heavy chain variable domain comprising the CDR-H1 of SEQ ID NO: 12, the CDR-H2 of SEQ ID NO: 28, and the CDR-H3 of SEQ ID NO: 44; (xiii) a heavy chain variable domain comprising the CDR-H1 of SEQ ID NO: 13, the CDR-H2 of SEQ ID NO: 29, and the CDR-H3 of SEQ ID NO: 45; (xiv) a heavy chain variable domain comprising the CDR-H1 of SEQ ID NO: 14, the CDR-H2 of SEQ ID NO: 30, and the CDR-H3 of SEQ ID NO: 46; (xv) a heavy chain variable domain comprising the CDR-H1 of SEQ ID NO: 15, the CDR-H2 of SEQ ID NO: 31, and the CDR-H3 of SEQ ID NO: 47; (xvi) a heavy chain variable domain comprising the CDR-H1 of SEQ ID NO: 16, the CDR-H2 of SEQ ID NO: 32, and the CDR-H3 of SEQ ID NO: 48; (xvii) a heavy chain variable domain comprising the CDR-H1 of SEQ ID NO: 135, the CDR-H2 of SEQ ID NO: 137, and the CDR-H3 of SEQ ID NO: 139; and (xviii) a heavy chain variable domain comprising the CDR-H1 of SEQ ID NO: 136, the CDR-H2 of SEQ ID NO: 138, and the CDR-H3 of SEQ ID NO: 140. The sequences of the heavy chain CDR sequences are provided in Table 1 below.

In an embodiment, the antibody-based molecule that binds to human muscle-specific tyrosine-protein kinase (MuSK) comprises: (ii.a) a heavy chain variable domain comprising the CDR-H1 of SEQ ID NO: 2, the CDR-H2 of SEQ ID NO: 18, and the CDR-H3 of SEQ ID NO: 240 (X2m1); (ii.b) a heavy chain variable domain comprising the CDR-H1 of SEQ ID NO: 2, the CDR-H2 of SEQ ID NO: 18, and the CDR-H3 of SEQ ID NO: 241 (X2m2); (ii.c) a heavy chain variable domain comprising the CDR-H1 of SEQ ID NO: 2, the CDR-H2 of SEQ ID NO: 18, and the CDR-H3 of SEQ ID NO: 242 (X2m3); (ii.d) a heavy chain variable domain comprising the CDR-H1 of SEQ ID NO: 2, the CDR-H2 of SEQ ID NO: 18, and the CDR-H3 of SEQ ID NO: 243 (X2m4); (ii.e) a heavy chain variable domain comprising the CDR-H1 of SEQ ID NO: 2, the CDR-H2 of SEQ ID NO: 18, and the CDR-H3 of SEQ ID NO: 244 (X2m5); (ii.f) a heavy chain variable domain comprising the CDR-H1 of SEQ ID NO: 2, the CDR-H2 of SEQ ID NO: 18, and the CDR-H3 of SEQ ID NO: 245 (X2m6); (ii.g) a heavy chain variable domain comprising the CDR-H1 of SEQ ID NO: 2, the CDR-H2 of SEQ ID NO: 18, and the CDR-H3 of SEQ ID NO: 246 (X2m7);
(ii.h) a heavy chain variable domain comprising the CDR-H1 of SEQ ID NO: 2, the CDR-H2 of SEQ ID NO: 18, and the CDR-H3 of SEQ ID NO: 247 (X2m8).

In an embodiment, the antibody-based molecule that binds to human muscle-specific tyrosine-protein kinase (MuSK) comprises: (xvii.a) a heavy chain variable domain comprising the CDR-H1 of SEQ ID NO: 135, the CDR-H2 of SEQ ID NO: 137, and the CDR-H3 of SEQ ID NO: 248 (X17m1); (xvii.b) a heavy chain variable domain comprising the CDR-H1 of SEQ ID NO: 135, the CDR-H2 of SEQ ID NO: 137, and the CDR-H3 of SEQ ID NO: 249(X17m2); (xvii.c) a heavy chain variable domain comprising the CDR-H1 of SEQ ID NO: 135, the CDR-H2 of SEQ ID NO: 137, and the CDR-H3 of SEQ ID NO: 250 (X17m3); (xvii.d) a heavy chain variable domain comprising the CDR-H1 of SEQ ID NO: 135, the CDR-H2 of SEQ ID NO: 137, and the CDR-H3 of SEQ ID NO: 251 (X17m6).

In an embodiment, the antibody-based molecule that binds to human muscle-specific tyrosine-protein kinase (MuSK) comprises a heavy chain variable domain, where the heavy chain variable domain comprises: (xix) a heavy chain variable domain comprising the CDR-H1 of SEQ ID NO: 147, the CDR-H2 of SEQ ID NO: 150, and the CDR-H3 of SEQ ID NO: 156; (xx) a heavy chain variable domain comprising the CDR-H1 of SEQ ID NO: 148, the CDR-H2 of SEQ ID NO: 151, and the CDR-H3 of SEQ ID NO: 157; (xxi) a heavy chain variable domain comprising the CDR-H1 of SEQ ID NO: 149, the CDR-H2 of SEQ ID NO: 152, and the CDR-H3 of SEQ ID NO: 158; In an embodiment, the antibody-based molecule that binds to human muscle-specific tyrosine-protein kinase (MuSK) comprises a heavy chain variable domain, where the heavy chain variable domain comprises (xxii) a heavy chain variable domain comprising the CDR-H1 of SEQ ID NO: 147, the CDR-H2 of SEQ ID NO: 153, and the CDR-H3 of SEQ ID NO:156 (3B2g1m1/3B2g2m1); (xxiii) a heavy chain variable domain comprising the CDR-H1 of SEQ ID NO: 147, the CDR-H2 of SEQ ID NO: 154, and the CDR-H3 of SEQ ID NO: 156 (3B2g1m2/3B2g2m2); (xxiv) a heavy chain variable domain comprising the CDR-H1 of SEQ ID NO: 147, the CDR-H2 of SEQ ID NO: 155, and the CDR-H3 of SEQ ID NO: 156 (3B2g1m4/3B2g2m4). The sequences of the heavy chain CDR sequences are provided in Table 1 below.

In an embodiment, the antibody-based molecule that binds to human muscle-specific tyrosine-protein kinase (MuSK) comprises a heavy chain variable domain, where the heavy chain variable domain comprises the CDR-H1 of SEQ ID NO: 147, CDR-H2 of SEQ ID NO: 153 or a CDR-H2 amino acid sequence having at least 0, 1, 2, 3, 4, or 5 alterations relative to SEQ ID NO: 153, and the CDR-H3 of SEQ ID NO:156 (3B2g2m1).

In an embodiment, the CDR-H2 amino acid sequence has at least 0, 1, 2, 3, 4, or 5 alterations relative to SEQ ID NO: 153. In accordance with this embodiment, the CDR-H2 amino acid sequence has at least 0, 1, 2, 3, 4, or 5 alterations relative to SEQ ID NO: 153, wherein said alterations are present at residues 1, 2, 6, 7, 8, 9, 10, 11, 12, 13, 14, 15, 16, 17, or any combination thereof.

In an embodiment, the antibody-based molecule that binds to human muscle-specific tyrosine-protein kinase (MUSK) comprises a heavy chain variable domain, where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO:156 or having 1, 2, 3, 4 or 5 amino acid alterations relative to SEQ ID NO:156 (3K2g2m1).

In an embodiment, the CDR-H2 of the antibody comprises a proline (P) at position 3, a tryptophan (W) at position 4, and a serine (5) or asparagine (N) at position 5.

In an embodiment, the antibody-based molecule that binds to human muscle-specific tyrosine-protein kinase (MUSK) comprises a heavy chain variable domain, where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (31B2g2m1).

The sequences of the heavy chain CDR sequences are provided in Table 1 below.

| | HCDR1 | | HCDR2 | | HCDR3 | |
|---|---|---|---|---|---|---|
| | | SEQ | | SEQ | | SEQ |
| mAb/Fab | | ID | | ID | | ID |
| name | Sequence | NO: | Sequence | NO: | Sequence | NO: |
| X1 | SSSIH | 1 | SISSSSGSTSYADSVKG | 17 | KYWSQYYWAHYYGGLDY | 33 |
| X2 | SSSIH | 2 | SISSSYGSTSYADSVKG | 18 | SEGDRYVSGYMGMDY | 34 |
| X2m1 | SSSIH | 2 | SISSSYGSTSYADSVKG | 18 | SEGDRYVSGYFGFDY | 240 |
| X2m2 | SSSIH | 2 | SISSSYGSTSYADSVKG | 18 | SEGDRYVSGYFGLDY | 241 |
| X2m3 | SSSIH | 2 | SISSSYGSTSYADSVKG | 18 | SEGDRYVSGYSGFDY | 242 |
| X2m4 | SSSIH | 2 | SISSSYGSTSYADSVKG | 18 | SEGDRYVSGYSGLDY | 243 |
| X2m5 | SSSIH | 2 | SISSSYGSTSYADSVKG | 18 | SEGDRYVSGYFGMDY | 244 |
| X2m6 | SSSIH | 2 | SISSSYGSTSYADSVKG | 18 | SEGDRYVSGYSGMDY | 245 |
| X2m7 | SSSIH | 2 | SISSSYGSTSYADSVKG | 18 | SEGDRYVSGYMGFDY | 246 |
| X2m8 | SSSIH | 2 | SISSSYGSTSYADSVKG | 18 | SEGDRYVSGYMGLDY | 247 |
| X3 | SSSIH | 3 | SISSSSGYTYYADSVKG | 19 | SWYEMWMSGYFGFDY | 35 |
| X4 | SSSIH | 4 | SISSSSGSTYYADSVKG | 20 | GEHDYYVFGYLGMDY | 36 |
| X5 | SSSIH | 5 | SISSSSGSTSYADSVKG | 21 | SYTMFYYGGWYGSGYFGM | 37 |
| | | | | | DY | |
| X6 | SSSIH | 6 | SISSYSGYTYYADSVKG | 22 | TYGSYYVSSYTGMDY | 38 |
| X7 | SSSIH | 7 | SISSSYSSTYYADSVKG | 23 | LAGLYHYPGYLGLDY | 39 |
| X8 | SSSIH | 8 | SISSSSGSTSYADSVKG | 24 | SWSYHPWYYHVGWYTGLD | 40 |
| | | | | | Y | |
| X9 | SSSIH | 9 | SIYSSSGSTYYADSVKG | 25 | SGGEFYITSYYGMDY | 41 |
| X10 | SSSIH | 10 | SISSSYSSTSYADSVKG | 26 | KYYRWRHNKYQGFDY | 42 |
| X11 | SSSIH | 11 | SISSYSGSTYYADSVKG | 27 | SWGSYYVSGFVGFDY | 43 |
| X12 | SSSIH | 12 | YISPSSGYTSYADSVKG | 28 | QYWVPQWWITQYFGMDY | 44 |
| X13 | SSSIH | 13 | SISSSSGSTSYADSVKG | 29 | SSEHWYTIGYYGIDY | 45 |
| X14 | SSSIH | 14 | SISSSSGYTYYADSVKG | 30 | GSHHWFLWIYSGLDY | 46 |
| X15 | SSSIH | 15 | SISSSYGSTSYADSVKG | 31 | SEGDRYVSGYMGMDY | 47 |
| X16 | SSSIH | 16 | SIYSSYGYTSYADSVKG | 32 | NWGYYMYWGWYYALDY | 48 |
| X17 | YSSIH | 135 | SIYSSSGSTYYADSVKG | 137 | GDHGYYVFGYLGMDY | 139 |
| X17m1 | YSSIH | 135 | SIYSSSGSTYYADSVKG | 137 | GDHGYYVSGYLGMDY | 248 |
| X17m2 | YSSIH | 135 | SIYSSSGSTYYADSVKG | 137 | GDHGYYVYGYLGMDY | 249 |
| X17m3 | YSSIH | 135 | SIYSSSGSTYYADSVKG | 137 | GDHGYYVSGYLGFDY | 250 |
| X17m6 | YSSIH | 135 | SIYSSSGSTYYADSVKG | 137 | GEHGYYVSGYLGFDY | 251 |
| X18 | SSSIH | 136 | SISSSSGYTSYADSVKG | 138 | KYSKRAYPDYYWRGLDY | 140 |
| 14D10 | DYGMS | 147 | AIPWNGGSTYYKESVKG | 150 | RSGRIAFGALDA | 156 |
| 7G4 | DYGMS | 147 | AIPWNGGSTYYKESVKG | 150 | RSGRIAFGALDA | 156 |
| 3C4 | DYGMS | 147 | AIPWNGGSTYYKESVKG | 150 | RSGRIAFGALDA | 156 |
| 3B2 | DYGMS | 147 | AIPWNGGSTYYKESVKG | 150 | RSGRIAFGALDA | 156 |
| 3G3 | DYGMS | 147 | AIPWNGGSTYYKESVKG | 150 | RSGRIAFGALDA | 156 |
| 31G2 | DYGMS | 147 | AIPWNGGSTYYKESVKG | 150 | RSGRIAFGALDA | 156 |
| 31B7 | DYGMS | 147 | AIPWNGGSTYYKESVKG | 150 | RSGRIAFGALDA | 156 |
| 17H10 | ARYYS | 148 | VIAYDGSTYYSPSLKS | 151 | GSSRVAAAFDS | 157 |
| | WS | | | | | |
| 23B6 | ARYYS | 148 | VIAYDGSTYYSPSLKS | 151 | GSSRVAAAFDS | 157 |
| | WS | | | | | |
| 30E1 | ARYYS | 148 | VIAYDGSTYYSPSLKS | 151 | GSSRVAAAFDS | 157 |
| | WS | | | | | |
| 30A11 | ARYYS | 148 | VIAYDGSTYYSPSLKS | 151 | GSSRVAAAFDS | 157 |
| | WS | | | | | |
| 16F11 | LYYMN | 149 | VIDTHSIAYYADSVKG | 152 | GRTALVR | 158 |
| 4C11 | LYYMN | 149 | VIDTHSIAYYADSVKG | 152 | GRTALVR | 158 |
| 7A12 | LYYMN | 149 | VIDTHSIAYYADSVKG | 152 | GRTALVR | 158 |
| 7G12 | LYYMN | 149 | VIDTHSIAYYADSVKG | 152 | GRTALVR | 158 |
| 7B8 | LYYMN | 149 | VIDTHSIAYYADSVKG | 152 | GRTALVR | 158 |
| 3B2g1m1 | DYGMS | 147 | AIPWSGGSTYYKESVKG | 153 | RSGRIAFGALDA | 156 |
| 3B2g1m2 | DYGMS | 147 | AIPGSGGSTYYKESVKG | 154 | RSGRIAFGALDA | 156 |
| 3B2g1m4 | DYGMS | 147 | AIPWQGGSTYYKESVKG | 155 | RSGRIAFGALDA | 156 |
| 3B2g2m1 | DYGMS | 147 | AIPWSGGSTYYKESVKG | 153 | RSGRIAFGALDA | 156 |
| 3B2g2m2 | DYGMS | 147 | AIPGSGGSTYYKESVKG | 154 | RSGRIAFGALDA | 156 |
| 3B2g2m4 | DYGMS | 147 | AIPWQGGSTYYKESVKG | 155 | RSGRIAFGALDA | 156 |

In some embodiments, the MuSK antibody-based molecules as disclosed herein further comprise a light chain variable domain. The light chain variable domain comprises
(i) a complementarity-determining region 1 (CDR-L1) having an amino acid sequence of any one of SEQ ID NOs: 49-64, 141, 142, 159-169, or a modified amino acid sequence of any one of SEQ ID NO: 49-64, 141, 142, or 159-169, said modified sequence having at least 80% sequence identity to any one of SEQ ID NO: 49-64, 141, 142, or 159-169;
(ii) a complementarity-determining region 2 (CDR-L2) having an amino acid sequence of any one of SEQ ID NOs: 65-80, 143, 144, 170-179, or a modified amino acid sequence of any one of SEQ ID NO: 65-80, 143, 144 or 170-179, said modified sequence having at least 80% sequence identity to any one of SEQ ID NO: 65-80, 143, 144 or 170-179; and
(iii) a complementarity-determining region 3 (CDR-L3) having an amino acid sequence of any one of SEQ ID NOs: 81-96, 145, 146, 180-195, or a modified amino acid sequence of any one of SEQ ID NO: 81-96, 145, 146, or 180-195, said modified sequence having at least 80% sequence identity to any one of SEQ ID NO: 81-96, 145, 146 or 180-195.

In some embodiments, the MuSK antibody-based molecules as disclosed herein further comprise a light chain variable domain. The light chain variable domain comprises
(iv) a complementarity-determining region 1 (CDR-L1) having an amino acid sequence of any one of SEQ ID NOs: 49-64, 141, 142, 159-169, or a modified amino acid sequence of any one of SEQ ID NO: 49-64, 141, 142, or 159-169, said modified sequence having 1, 2, 3, 4 or 5 amino acid alterations relative to to any one of SEQ ID NO: 49-64, 141, 142, or 159-169;
(v) a complementarity-determining region 2 (CDR-L2) having an amino acid sequence of any one of SEQ ID NOs: 65-80, 143, 144, 170-179, or a modified amino acid sequence of any one of SEQ ID NO: 65-80, 143, 144 or 170-179, said modified sequence having 1, 2, 3, 4 or 5 amino acid alterations relative to any one of SEQ ID NO: 65-80, 143, 144 or 170-179; and
(vi) a complementarity-determining region 3 (CDR-L3) having an amino acid sequence of any one of SEQ ID NOs: 81-96, 145, 146, 180-195, or a modified amino acid sequence of any one of SEQ ID NO: 81-96, 145, 146, or 180-195, said modified sequence having 1, 2, 3, 4 or 5 amino acid alterations relative to any one of SEQ ID NO: 81-96, 145, 146 or 180-195.

In an embodiment, the light chain variable domain of the MuSK antibody based molecule disclosed herein comprises (i) a light chain variable domain comprising the CDR-L1 of SEQ ID NO: 49 or having 1, 2, 3, 4 or 5 amino acid alterations relative to SEQ ID NO:49, the CDR-L2 of SEQ ID NO: 65 or having 1, 2, 3, 4 or 5 amino acid alterations relative to SEQ ID NO:65, and the CDR-L3 of SEQ ID NO: 81 or having 1, 2, 3, 4 or 5 amino acid alterations relative to SEQ ID NO:81; (ii) a light chain variable domain comprising the CDR-L1 of SEQ ID NO: 50 or having 1, 2, 3, 4 or 5 amino acid alterations relative to SEQ ID NO:50, the CDR-L2 of SEQ ID NO: 66 or having 1, 2, 3, 4 or 5 amino acid alterations relative to SEQ ID NO:66, and the CDR-L3 of SEQ ID NO: 82 or having 1, 2, 3, 4 or 5 amino acid alterations relative to SEQ ID NO:82; (iii) a light chain variable domain comprising the CDR-L1 of SEQ ID NO: 51 or having 1, 2, 3, 4 or 5 amino acid alterations relative to SEQ ID NO:51, the CDR-L2 of SEQ ID NO: 67 or having 1, 2, 3, 4 or 5 amino acid alterations relative to SEQ ID NO:67, and the CDR-L3 of SEQ ID NO: 83 or having 1, 2, 3, 4 or 5 amino acid alterations relative to SEQ ID NO:83; (iv) a light chain variable domain comprising the CDR-L1 of SEQ ID NO: 52 or having 1, 2, 3, 4 or 5 amino acid alterations relative to SEQ ID NO:52, the CDR-L2 of SEQ ID NO: 68 or having 1, 2, 3, 4 or 5 amino acid alterations relative to SEQ ID NO:68, and the CDR-L3 of SEQ ID NO: 84 or having 1, 2, 3, 4 or 5 amino acid alterations relative to SEQ ID NO:84; (v) a light chain variable domain comprising the CDR-L1 of SEQ ID NO: 53 or having 1, 2, 3, 4 or 5 amino acid alterations relative to SEQ ID NO:53, the CDR-L2 of SEQ ID NO: 69 or having 1, 2, 3, 4 or 5 amino acid alterations relative to SEQ ID NO:69, and the CDR-L3 of SEQ ID NO: 85 or having 1, 2, 3, 4 or 5 amino acid alterations relative to SEQ ID NO:85; (vi) a light chain variable domain comprising the CDR-L1 of SEQ ID NO: 54 or having 1, 2, 3, 4 or 5 amino acid alterations relative to SEQ ID NO:54, the CDR-L2 of SEQ ID NO: 70 or having 1, 2, 3, 4 or 5 amino acid alterations relative to SEQ ID NO:70, and the CDR-L3 of SEQ ID NO: 86 or having 1, 2, 3, 4 or 5 amino acid alterations relative to SEQ ID NO:86; (vii) a light chain variable domain comprising the CDR-L1 of SEQ ID NO: 55 or having 1, 2, 3, 4 or 5 amino acid alterations relative to SEQ ID NO:55, the CDR-L2 of SEQ ID NO: 71 or having 1, 2, 3, 4 or 5 amino acid alterations relative to SEQ ID NO:71, and the CDR-L3 of SEQ ID NO: 87 or having 1, 2, 3, 4 or 5 amino acid alterations relative to SEQ ID NO:87; (viii) a light chain variable domain comprising the CDR-L1 of SEQ ID NO: 56 or having 1, 2, 3, 4 or 5 amino acid alterations relative to SEQ ID NO:56, the CDR-L2 of SEQ ID NO: 72 or having 1, 2, 3, 4 or 5 amino acid alterations relative to SEQ ID NO:72, and the CDR-L3 of SEQ ID NO: 88 or having 1, 2, 3, 4 or 5 amino acid alterations relative to SEQ ID NO:88; (ix) a light chain variable domain comprising the CDR-L1 of SEQ ID NO: 57 or having 1, 2, 3, 4 or 5 amino acid alterations relative to SEQ ID NO:57, the CDR-L2 of SEQ ID NO: 73 or having 1, 2, 3, 4 or 5 amino acid alterations relative to SEQ ID NO:73, and the CDR-L3 of SEQ ID NO: 89 or having 1, 2, 3, 4 or 5 amino acid alterations relative to SEQ ID NO:89; (x) a light chain variable domain comprising the CDR-L1 of SEQ ID NO: 58 or having 1, 2, 3, 4 or 5 amino acid alterations relative to SEQ ID NO:58, the CDR-L2 of SEQ ID NO: 74 or having 1, 2, 3, 4 or 5 amino acid alterations relative to SEQ ID NO:74, and the CDR-L3 of SEQ ID NO: 90 or having 1, 2, 3, 4 or 5 amino acid alterations relative to SEQ ID NO:90; (xi) a light chain variable domain comprising the CDR-L1 of SEQ ID NO: 59 or having 1, 2, 3, 4 or 5 amino acid alterations relative to SEQ ID NO:59, the CDR-L2 of SEQ ID NO: 75 or having 1, 2, 3, 4 or 5 amino acid alterations relative to SEQ ID NO:75, and the CDR-L3 of SEQ ID NO: 91 or having 1, 2, 3, 4 or 5 amino acid alterations relative to SEQ ID NO:91; (xii) a light chain variable domain comprising the CDR-L1 of SEQ ID NO: 60 or having 1, 2, 3, 4 or 5 amino acid alterations relative to SEQ ID NO:60, the CDR-L2 of SEQ ID NO: 76 or having 1, 2, 3, 4 or 5 amino acid alterations relative to SEQ ID NO:76, and the CDR-L3 of SEQ ID NO: 92 or having 1, 2, 3, 4 or 5 amino acid alterations relative to SEQ ID NO:92; (xiii) a light chain variable domain comprising the CDR-L1 of SEQ ID NO: 61 or having 1, 2, 3, 4 or 5 amino acid alterations relative to SEQ ID NO:61, the CDR-L2 of SEQ ID NO: 77 or having 1, 2, 3, 4 or 5 amino acid alterations relative to SEQ ID NO:77, and the CDR-L3 of SEQ ID NO: 93 or having 1, 2, 3, 4 or 5 amino acid alterations relative to SEQ ID NO:93; (xiv) a light chain variable domain comprising the CDR-L1 of SEQ ID NO: 62 or having 1, 2, 3, 4 or 5 amino acid alterations relative to SEQ ID NO:62, the CDR-L2 of SEQ ID NO: 78 or having 1, 2, 3, 4 or amino acid alterations relative to SEQ ID NO:78, and the CDR-L3 of SEQ ID NO: 94 or having 1, 2, 3, 4 or 5 amino acid alterations relative to SEQ ID NO:94; (xv) a light chain variable domain comprising the CDR-L1 of SEQ ID NO: 63 or having 1, 2, 3, 4 or 5 amino acid alterations relative to SEQ ID NO:63, the CDR-L2 of SEQ ID NO: 79 or having 1, 2, 3, 4 or 5 amino acid alterations relative to SEQ ID NO:79, and the CDR-L3 of SEQ ID NO: 95 or having 1, 2, 3, 4 or 5 amino acid alterations relative to SEQ ID NO:95; (xvi) a light chain variable domain comprising the CDR-L1 of SEQ ID NO: 64 or having 1, 2, 3, 4 or 5 amino acid alterations relative to SEQ ID NO:64, the CDR-L2 of SEQ ID NO: 80 or having 1, 2, 3, 4 or 5 amino acid alterations relative to SEQ ID NO:80, and the CDR-L3 of SEQ ID NO: 96 or having 1, 2, 3, 4 or 5 amino acid alterations relative to SEQ ID NO:96; (xvii) a light chain variable domain comprising the CDR-L1 of SEQ ID NO: 141 or having 1, 2, 3, 4 or 5 amino acid alterations relative to SEQ ID NO:141, the CDR-L2 of SEQ ID NO: 143 or having 1, 2, 3, 4 or 5 amino acid alterations relative to SEQ ID NO:143, and the CDR-L3 of SEQ ID NO: 145 or having 1, 2, 3, 4 or 5 amino acid alterations relative to SEQ ID NO:145; (xviii) a light chain variable domain comprising the CDR-L1 of SEQ ID NO: 142 or having 1, 2, 3, 4 or 5 amino acid alterations relative to SEQ ID NO:142, the CDR-L2 of SEQ ID NO: 144 or having 1, 2, 3, 4 or 5 amino acid alterations relative to SEQ ID NO:144, and the CDR-L3 of SEQ ID NO: 146 or having 1, 2, 3, 4 or 5 amino acid alterations relative to SEQ ID NO:146. The sequences of the light chain CDRs are provided in Table 2 below.

In an embodiment, the light chain variable domain of the MuSK antibody based molecule disclosed herein comprises (xix) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 170 or having 1, 2, 3, 4 or 5 amino acid alterations relative to SEQ ID NO:170, and the CDR-L3 of SEQ ID NO: 180 or having 1, 2, 3, 4 or 5 amino acid alterations relative to SEQ ID NO:180; (xx) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 171 or having 1, 2, 3, 4 or 5 amino acid alterations relative to SEQ ID NO:171, and the CDR-L3 of SEQ ID NO: 181 or having 1, 2, 3, 4 or amino acid alterations relative to SEQ ID NO:181; (xxi) a light chain variable domain comprising the CDR-L1 of SEQ ID NO: 160 or having 1, 2, 3, 4 or 5 amino acid alterations relative to SEQ ID NO:160, the CDR-L2 of SEQ ID NO: 172 or having 1, 2, 3, 4 or 5 amino acid alterations relative to SEQ ID NO:172, and the CDR-L3 of SEQ ID NO: 182 or having 1, 2, 3, 4 or 5 amino acid alterations relative to SEQ ID NO:182; (xxii) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 172 or having 1, 2, 3, 4 or 5 amino acid alterations relative to SEQ ID NO:172, and the CDR-L3 of SEQ ID NO: 183 or having 1, 2, 3, 4 or 5 amino acid alterations relative to SEQ ID NO:183; (xxiii) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 171 or having 1, 2, 3, 4 or 5 amino acid alterations relative to SEQ ID NO:171, and the CDR-L3 of SEQ ID NO: 184 or having 1, 2, 3, 4 or amino acid alterations relative to SEQ ID NO:184; (xxiv) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 173 or having 1, 2, 3, 4 or 5 amino acid alterations relative to SEQ ID NO:173, and the CDR-L3 of SEQ ID NO: 185 or having 1, 2, 3, 4 or 5 amino acid alterations relative to SEQ ID NO:185; (xxv) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 173 or having 1, 2, 3, 4 or 5 amino acid alterations relative to SEQ ID NO:173, and the CDR-L3 of SEQ ID NO: 186 or having 1, 2, 3, 4 or 5 amino acid alterations relative to SEQ ID NO:186; (xxvi) a light chain variable domain comprising the CDR-L1 of SEQ ID NO: 161 or having 1, 2, 3, 4 or 5 amino acid alterations relative to SEQ ID NO:161, the CDR-L2 of SEQ ID NO: 174 or having 1, 2, 3, 4 or 5 amino acid alterations relative to SEQ ID NO:174, and the CDR-L3 of SEQ ID NO: 187 or having 1, 2, 3, 4 or amino acid alterations relative to SEQ ID NO:187; (xxvii) a light chain variable domain comprising the CDR-L1 of SEQ ID NO: 162 or having 1, 2, 3, 4 or 5 amino acid alterations relative to SEQ ID NO:162, the CDR-L2 of SEQ ID NO: 174 or having 1, 2, 3, 4 or 5 amino acid alterations relative to SEQ ID NO:174, and the CDR-L3 of SEQ ID NO: 188 or having 1, 2, 3, 4 or 5 amino acid alterations relative to SEQ ID NO:188; (xxviii) a light chain variable domain comprising the CDR-L1 of SEQ ID NO: 163 or having 1, 2, 3, 4 or 5 amino acid alterations relative to SEQ ID NO:163, the CDR-L2 of SEQ ID NO: 174 or having 1, 2, 3, 4 or 5 amino acid alterations relative to SEQ ID NO:174, and the CDR-L3 of SEQ ID NO: 188 or having 1, 2, 3, 4 or 5 amino acid alterations relative to SEQ ID NO:188; (xxix) a light chain variable domain comprising the CDR-L1 of SEQ ID NO: 164 or having 1, 2, 3, 4 or 5 amino acid alterations relative to SEQ ID NO:164, the CDR-L2 of SEQ ID NO: 174 or having 1, 2, 3, 4 or 5 amino acid alterations relative to SEQ ID NO:174, and the CDR-L3 of SEQ ID NO: 189 or having 1, 2, 3, 4 or amino acid alterations relative to SEQ ID NO:189; (xxx) a light chain variable domain comprising the CDR-L1 of SEQ ID NO: 165 or having 1, 2, 3, 4 or 5 amino acid alterations relative to SEQ ID NO:165, the CDR-L2 of SEQ ID NO: 175 or having 1, 2, 3, 4 or 5 amino acid alterations relative to SEQ ID NO:175, and the CDR-L3 of SEQ ID NO: 190 or having 1, 2, 3, 4 or 5 amino acid alterations relative to SEQ ID NO:190; (xxxi) a light chain variable domain comprising the CDR-L1 of SEQ ID NO: 166 or having 1, 2, 3, 4 or 5 amino acid alterations relative to SEQ ID NO:166, the CDR-L2 of SEQ ID NO: 176 or having 1, 2, 3, 4 or 5 amino acid alterations relative to SEQ ID NO:176, and the CDR-L3 of SEQ ID NO: 191 or having 1, 2, 3, 4 or 5 amino acid alterations relative to SEQ ID NO:191; (xxxi) a light chain variable domain comprising the CDR-L1 of SEQ ID NO: 167 or having 1, 2, 3, 4 or 5 amino acid alterations relative to SEQ ID NO:167, the CDR-L2 of SEQ ID NO: 177 or having 1, 2, 3, 4 or 5 amino acid alterations relative to SEQ ID NO:177, and the CDR-L3 of SEQ ID NO: 192 or having 1, 2, 3, 4 or amino acid alterations relative to SEQ ID NO:192; (xxxii) a light chain variable domain comprising the CDR-L1 of SEQ ID NO: 168 or having 1, 2, 3, 4 or 5 amino acid alterations relative to SEQ ID NO:168, the CDR-L2 of SEQ ID NO: 178 or having 1, 2, 3, 4 or 5 amino acid alterations relative to SEQ ID NO:178, and the CDR-L3 of SEQ ID NO: 193 or having 1, 2, 3, 4 or 5 amino acid alterations relative to SEQ ID NO:193; (xxxiii) a light chain variable domain comprising the CDR-L1 of SEQ ID NO: 169 or having 1, 2, 3, 4 or 5 amino acid alterations relative to SEQ ID NO:169, the CDR-L2 of SEQ ID NO: 179 or having 1, 2, 3, 4 or 5 amino acid alterations relative to SEQ ID NO:179, and the CDR-L3 of SEQ ID NO: 194 or having 1, 2, 3, 4 or 5 amino acid alterations relative to SEQ ID NO:194.

In an embodiment, the light chain variable domain of the MuSK antibody based molecule disclosed herein comprises (i) a light chain variable domain comprising the CDR-L1 of SEQ ID NO: 49, the CDR-L2 of SEQ ID NO: 65, and the CDR-L3 of SEQ ID NO: 81; (ii) a light chain variable domain comprising the CDR-L1 of SEQ ID NO: 50, the CDR-L2 of SEQ ID NO: 66, and the CDR-L3 of SEQ ID NO: 82; (iii) a light chain variable domain comprising the CDR-L1 of SEQ ID NO: 51, the CDR-L2 of SEQ ID NO: 67, and the CDR-L3 of SEQ ID NO: 83; (iv) a light chain variable domain comprising the CDR-L1 of SEQ ID NO: 52, the CDR-L2 of SEQ ID NO: 68, and the CDR-L3 of SEQ ID NO: 84; (v) a light chain variable domain comprising the CDR-L1 of SEQ ID NO: 53, the CDR-L2 of SEQ ID NO: 69, and the CDR-L3 of SEQ ID NO: 85; (vi) a light chain variable domain comprising the CDR-L1 of SEQ ID NO: 54, the CDR-L2 of SEQ ID NO: 70, and the CDR-L3 of SEQ ID NO: 86; (vii) a light chain variable domain comprising the CDR-L1 of SEQ ID NO: 55, the CDR-L2 of SEQ ID NO: 71, and the CDR-L3 of SEQ ID NO: 87; (viii) a light chain variable domain comprising the CDR-L1 of SEQ ID NO: 56, the CDR-L2 of SEQ ID NO: 72, and the CDR-L3 of SEQ ID NO: 88; (ix) a light chain variable domain comprising the CDR-L1 of SEQ ID NO: 57, the CDR-L2 of SEQ ID NO: 73, and the CDR-L3 of SEQ ID NO: 89; (x) a light chain variable domain comprising the CDR-L1 of SEQ ID NO: 58, the CDR-L2 of SEQ ID NO: 74, and the CDR-L3 of SEQ ID NO: 90; (xi) a light chain variable domain comprising the CDR-L1 of SEQ ID NO: 59, the CDR-L2 of SEQ ID NO: 75, and the CDR-L3 of SEQ ID NO: 91; (xii) a light chain variable domain comprising the CDR-L1 of SEQ ID NO: 60, the CDR-L2 of SEQ ID NO: 76, and the CDR-L3 of SEQ ID NO: 92; (xiii) a light chain variable domain comprising the CDR-L1 of SEQ ID NO: 61, the CDR-L2 of SEQ ID NO: 77, and the CDR-L3 of SEQ ID NO: 93; (xiv) a light chain variable domain comprising the CDR-L1 of SEQ ID NO: 62, the CDR-L2 of SEQ ID NO: 78, and the CDR-L3 of SEQ ID NO: 94; (xv) a light chain variable domain comprising the CDR-L1 of SEQ ID NO: 63, the CDR-L2 of SEQ ID NO: 79, and the CDR-L3 of SEQ ID NO: 95; (xvi) a light chain variable domain comprising the CDR-L1 of SEQ ID NO: 64, the CDR-L2 of SEQ ID NO: 80, and the CDR-L3 of SEQ ID NO: 96; (xvii) a light chain variable domain comprising the CDR-L1 of SEQ ID NO: 141, the CDR-L2 of SEQ ID NO: 143, and the CDR-L3 of SEQ ID NO: 145; (xviii) a light chain variable domain comprising the CDR-L1 of SEQ ID NO: 142, the CDR-L2 of SEQ ID NO: 144, and the CDR-L3 of SEQ ID NO: 146. The sequences of the light chain CDRs are provided in Table 2 below.

In an embodiment, the light chain variable domain of the MuSK antibody based molecule disclosed herein comprises (xix) a light chain variable domain comprising the CDR-L1 of SEQ ID NO: 159, the CDR-L2 of SEQ ID NO: 170, and the CDR-L3 of SEQ ID NO: 180; (xx) a light chain variable domain comprising the CDR-L1 of SEQ ID NO: 159, the CDR-L2 of SEQ ID NO: 171, and the CDR-L3 of SEQ ID NO: 181; (xxi) a light chain variable domain comprising the CDR-L1 of SEQ ID NO: 160, the CDR-L2 of SEQ ID NO: 172, and the CDR-L3 of SEQ ID NO: 182; (xxii) a light chain variable domain comprising the CDR-L1 of SEQ ID NO: 159, the CDR-L2 of SEQ ID NO: 172, and the CDR-L3 of SEQ ID NO: 183; (xxiii) a light chain variable domain comprising the CDR-L1 of SEQ ID NO: 159, the CDR-L2 of SEQ ID NO: 171, and the CDR-L3 of SEQ ID NO: 184; (xxiv) a light chain variable domain comprising the CDR-L1 of SEQ ID NO: 159, the CDR-L2 of SEQ ID NO: 173, and the CDR-L3 of SEQ ID NO: 185; (xxv) a light chain variable domain comprising the CDR-L1 of SEQ ID NO: 159, the CDR-L2 of SEQ ID NO: 173, and the CDR-L3 of SEQ ID NO: 186; (xxvi) a light chain variable domain comprising the CDR-L1 of SEQ ID NO: 161, the CDR-L2 of SEQ ID NO: 174, and the CDR-L3 of SEQ ID NO: 187; (xxvii) a light chain variable domain comprising the CDR-L1 of SEQ ID NO: 162, the CDR-L2 of SEQ ID NO: 174, and the CDR-L3 of SEQ ID NO: 188; (xxviii) a light chain variable domain comprising the CDR-L1 of SEQ ID NO: 163, the CDR-L2 of SEQ ID NO: 174, and the CDR-L3 of SEQ ID NO: 188; (xxix) a light chain variable domain comprising the CDR-L1 of SEQ ID NO: 164, the CDR-L2 of SEQ ID NO: 174, and the CDR-L3 of SEQ ID NO: 189; (xxx) a light chain variable domain comprising the CDR-L1 of SEQ ID NO: 165, the CDR-L2 of SEQ ID NO: 175, and the CDR-L3 of SEQ ID NO: 190; (xxxi) a light chain variable domain comprising the CDR-L1 of SEQ ID NO: 166, the CDR-L2 of SEQ ID NO: 176, and the CDR-L3 of SEQ ID NO: 191; (xxxi) a light chain variable domain comprising the CDR-L1 of SEQ ID NO: 167, the CDR-L2 of SEQ ID NO: 177, and the CDR-L3 of SEQ ID NO: 192; (xxxii) a light chain variable domain comprising the CDR-L1 of SEQ ID NO: 168, the CDR-L2 of SEQ ID NO: 178, and the CDR-L3 of SEQ ID NO: 193; (xxxiii) a light chain variable domain comprising the CDR-L1 of SEQ ID NO: 169, the CDR-L2 of SEQ ID NO: 179, and the CDR-L3 of SEQ ID NO: 194.

In an embodiment, the light chain variable domain of the MuSK antibody based molecule disclosed herein comprises the CDR-L1 of SEQ ID NO: 159, the CDR-L2 of SEQ ID NO: 172, and the CDR-L3 of SEQ ID NO: 195 or a CDR-L3 having 1, 2, 3, 4 or 5 amino acid alterations relative to the amino acid sequence of SEQ ID NO: 195, wherein said alteration is present at residue 1, 2, 6, 7, 8, 9, 10, 11, 12, 13, 14, 15, 16, 17, or any combination thereof.

In an embodiment, the antibody-based molecule that binds to human muscle-specific tyrosine-protein kinase (MuSK) comprises a light chain variable domain, where the light chain variable domain comprises:
a CDR-L1 amino acid sequence comprising SEQ ID NO:159 or having 1, 2, 3, 4 or 5 amino acid alternations relative to SEQ ID NO: 159,
a CDR-L2 amino acid sequence comprising SEQ ID NO: 172 or having 1, 2, 3, 4 or 5 amino acid alternations relative to SEQ ID NO: 172, and
a CDR-L3 amino acid sequence comprising SEQ ID NO: 195 or having 1, 2, 3, 4 or 5 amino acid alternations relative SEQ ID NO:195 (3B2g2m1).

In an embodiment, the CDR-L1, CDR-L2, CDR-L3 amino acid sequence has at least 0, 1, 2, 3, 4 or 5 amino acid alternations relative to SEQ ID NO: 159, 172 or 195 (respectively).

In an embodiment, the antibody-based molecule that binds to human muscle-specific tyrosine-protein kinase (MuSK) comprises a light chain variable domain, where the light chain variable domain comprises:
a CDR-L1 amino acid sequence comprising or consisting of SEQ ID NO: 159,
a CDR-L2 amino acid sequence comprising or consisting of SEQ ID NO: 172, and a CDR-L3 amino acid sequence comprising or consisting of SEQ ID NO:195 (3B2g2m1).

The sequences of the light chain CDR sequences are provided in Table 2 below.

| | LCDR1 | | LCDR2 | | LCDR3 | |
|---|---|---|---|---|---|---|
| | | SEQ | | SEQ | | SEQ |
| mAb/Fab | | ID | | ID | | ID |
| name | Sequence | NO: | Sequence | NO: | Sequence | NO: |
| X1 | RASQSVSSAVA | 49 | SASSLYS | 65 | QQSSSSLIT | 81 |
| X2 | RASQSVSSAVA | 50 | SASSLYS | 66 | QQSGVWLIT | 82 |
| X3 | RASQSVSSAVA | 51 | SASSLYS | 67 | QQSSSSLIT | 83 |
| X4 | RASQSVSSAVA | 52 | SASSLYS | 68 | QQSYKPGALIT | 84 |
| X5 | RASQSVSSAVA | 53 | SASSLYS | 69 | QQSSSSLIT | 85 |
| X6 | RASQSVSSAVA | 54 | SASSLYS | 70 | QQSSSSLIT | 86 |
| X7 | RASQSVSSAVA | 55 | SASSLYS | 71 | QQSSRSSLLT | 87 |
| X8 | RASQSVSSAVA | 56 | SASSLYS | 72 | QQSSSSLIT | 88 |
| X9 | RASQSVSSAVA | 57 | SASSLYS | 73 | QQSSSSLIT | 89 |
| X10 | RASQSVSSAVA | 58 | SASSLYS | 74 | QQSLWYPVT | 90 |
| X11 | RASQSVSSAVA | 59 | SASSLYS | 75 | QQNSYYLIT | 91 |
| X12 | RASQSVSSAVA | 60 | SASSLYS | 76 | QQSSSSLIT | 92 |
| X13 | RASQSVSSAVA | 61 | SASSLYS | 77 | QQSYGSFSLIT | 93 |
| X14 | RASQSVSSAVA | 62 | SASSLYS | 78 | QQGSYHLIT | 94 |
| X15 | RASQSVSSAVA | 63 | SASSLYS | 79 | QQSGVWLIT | 95 |
| X16 | RASQSVSSAVA | 64 | SASSLYS | 80 | QQWSSAQALIT | 96 |
| X17 | RASQSVSSAVA | 141 | SASSLYS | 143 | QQSYKPGALIT | 145 |
| X18 | RASQSVSSAVA | 142 | SASSLYS | 144 | QQSYWWPIT | 146 |
| 14D10 | GLSSGSVTSSNYPD | 159 | TTNSRHS | 170 | ALYMGGGSNVYV | 180 |
| 7G4 | GLSSGSVTSSNYPD | 159 | STNSRHS | 171 | ALYMGRGSNKDYV | 181 |
| 3C4 | GLSSGSVTASNYPD | 160 | STDSRHS | 172 | ALYMYSDSKLYV | 182 |
| 3B2 | GLSSGSVTSSNYPD | 159 | STDSRHS | 172 | GLYMYSGSKNYV | 183 |
| 3G3 | GLSSGSVTSSNYPD | 159 | STNSRHS | 171 | ALYMGSDIRNYV | 184 |
| 31G2 | GLSSGSVTSSNYPD | 159 | STNSRLS | 173 | ALYMGSGSRNYV | 185 |
| 31B7 | GLSSGSVTSSNYPD | 159 | STNSRLS | 173 | ALYMGSESRNYV | 186 |
| 17H10 | GGNRIGGKSVQ | 161 | ADSRRPS | 174 | HVWGSTASAD | 187 |
| 23B6 | GGDNIGSKNAQ | 162 | ADSRRPS | 174 | HVWDSSTNAW | 188 |
| 30E1 | GGDNIGSKNTQ | 163 | ADSRRPS | 174 | HVWDSSTNAW | 188 |
| 30A11 | GGDNIASKNVQ | 164 | ADSRRPS | 174 | QVWDSSTNVAV | 189 |
| 16F11 | KSSQSVVFGSNQKSY | 165 | YASTQES | 175 | QQAYSAPT | 190 |
| | LN | | | | | |
| 4C11 | RSSQSVLYSSNQKNY | 166 | WASARES | 176 | QQSYKPPYG | 191 |
| | LN | | | | | |
| 7A12 | ESSQSVLYNQKNYLN | 167 | WASTRQS | 177 | QQAYNAPLT | 192 |
| 7G12 | KSSQRVQLGSNQKSY | 168 | YASTQQS | 178 | QQGYSAPFT | 193 |
| | LN | | | | | |
| 7B8 | KSSQSVLYNQKNYLA | 169 | WASTRES | 179 | QQGYSVPYT | 194 |
| 3B2g1m1 | GLSSGSVTSSNYPD | 159 | STDSRHS | 172 | GLYMYSGSKNYV | 183 |
| 3B2g1m2 | GLSSGSVTSSNYPD | 159 | STDSRHS | 172 | GLYMYSGSKNYV | 183 |
| 3B2g1m4 | GLSSGSVTSSNYPD | 159 | STDSRHS | 172 | GLYMYSGSKNYV | 183 |
| 3B2g2m1 | GLSSGSVTSSNYPD | 159 | STDSRHS | 172 | GLYSYSGSKNYV | 195 |
| 3B2g2m2 | GLSSGSVTSSNYPD | 159 | STDSRHS | 172 | GLYSYSGSKNYV | 195 |
| 3B2g2m4 | GLSSGSVTSSNYPD | 159 | STDSRHS | 172 | GLYSYSGSKNYV | 195 |

Suitable amino acid modifications to the heavy chain CDR sequences and/or the light chain CDR sequences of the MUSK antibody-based molecule disclosed herein include, for example, conservative substitutions or functionally equivalent amino acid residue substitutions that result in variant CDR sequences having similar or enhanced binding characteristics to those of the CDR sequences disclosed herein as described above. Encompassed by the present invention are CDRs of Tables 1 and 2 containing 1, 2, 3, 4, 5, or more amino acid alterations (depending on the length of the CDR) that maintain or enhance MUSK binding of the antibody. Suitable amino acid modifications to the heavy chain CDR sequences of Table 1 and/or the light chain CDR sequences of Tables 1 and 2 include, for example, conservative substitutions or functionally equivalent amino acid residue substitutions that result in variant CDR sequences having similar or enhanced binding characteristics to those of the CDR sequences of Table 1 and Table 2. Conservative substitutions are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally grouped separately as aliphatic-hydroxyl; (4) aromatic (phenylalanine, tyrosine, tryptophan); (5) amide (asparagine, glutamine); and (6) sulfur-containing (cysteine and methionine) (Stryer (ed.), Biochemistry, 2nd ed, WH Freeman and Co., 1981, which is hereby incorporated by reference in its entirety). Non-conservative substitutions can also be made to the heavy chain CDR sequences of Table 1 and the light chain CDR sequences of Table 2. Non-conservative substitutions involve substituting one or more amino acid residues of the CDR with one or more amino acid residues from a different class of amino acids to improve or enhance the binding properties of CDR. The amino acid sequences of the heavy chain CDRs of Table 1 and/or the light chain CDRs of Table 2 may further comprise one or more internal neutral amino acid insertions or deletions that maintain or enhance MuSK binding.

In an embodiment, the MuSK antibody-based molecule comprises:
(i) a heavy chain variable domain comprising the CDR-H1 of SEQ ID NO: 1, the CDR-H2 of SEQ ID NO: 17, and the CDR-H3 of SEQ ID NO: 33, and a light chain variable domain comprising the CDR-L1 of SEQ ID NO: 49, the CDR-L2 of SEQ ID NO: 65, and the CDR-L3 of SEQ ID NO: 81;
(ii) a heavy chain variable domain comprising the CDR-H1 of SEQ ID NO: 2, the CDR-H2 of SEQ ID NO: 18, and the CDR-H3 of SEQ ID NO: 34, and a light chain variable domain comprising the CDR-L1 of SEQ ID NO: 50, the CDR-L2 of SEQ ID NO: 66, and the CDR-L3 of SEQ ID NO: 82;
(iii) a heavy chain variable domain comprising the CDR-H1 of SEQ ID NO: 3, the CDR-H2 of SEQ ID NO: 19, and the CDR-H3 of SEQ ID NO: 35, and a light chain variable domain comprising the CDR-L1 of SEQ ID NO: 51, the CDR-L2 of SEQ ID NO: 67, and the CDR-L3 of SEQ ID NO: 83;
(iv) a heavy chain variable domain comprising the CDR-H1 of SEQ ID NO: 4, the CDR-H2 of SEQ ID NO: 20, and the CDR-H3 of SEQ ID NO: 36, and a light chain variable domain comprising the CDR-L1 of SEQ ID NO: 52, the CDR-L2 of SEQ ID NO: 68, and the CDR-L3 of SEQ ID NO: 84;
(v) a heavy chain variable domain comprising the CDR-H1 of SEQ ID NO: 5, the CDR-H2 of SEQ ID NO: 21, and the CDR-H3 of SEQ ID NO: 37, and a light chain variable domain comprising the CDR-L1 of SEQ ID NO: 53, the CDR-L2 of SEQ ID NO: 69, and the CDR-L3 of SEQ ID NO: 85;
(vi) a heavy chain variable domain comprising the CDR-H1 of SEQ ID NO: 6, the CDR-H2 of SEQ ID NO: 22, and the CDR-H3 of SEQ ID NO: 38, and a light chain variable domain comprising the CDR-L1 of SEQ ID NO: 54, the CDR-L2 of SEQ ID NO: 70, and the CDR-L3 of SEQ ID NO: 86;
(vii) a heavy chain variable domain comprising the CDR-H1 of SEQ ID NO: 7, the CDR-H2 of SEQ ID NO: 23, and the CDR-H3 of SEQ ID NO: 39, and a light chain variable domain comprising the CDR-L1 of SEQ ID NO: 55, the CDR-L2 of SEQ ID NO:71, and the CDR-L3 of SEQ ID NO: 87;
(viii) a heavy chain variable domain comprising the CDR-H1 of SEQ ID NO: 8, the CDR-H2 of SEQ ID NO: 24, and the CDR-H3 of SEQ ID NO: 40, and a light chain variable domain comprising the CDR-L1 of SEQ ID NO: 56, the CDR-L2 of SEQ ID NO: 72, and the CDR-L3 of SEQ ID NO: 88;
(ix) a heavy chain variable domain comprising the CDR-H1 of SEQ ID NO: 9, the CDR-H2 of SEQ ID NO: 25, and the CDR-H3 of SEQ ID NO: 41, and a light chain variable domain comprising the CDR-L1 of SEQ ID NO: 57, the CDR-L2 of SEQ ID NO: 73, and the CDR-L3 of SEQ ID NO: 89;
(x) a heavy chain variable domain comprising the CDR-H1 of SEQ ID NO: 10, the CDR-H2 of SEQ ID NO: 26, and the CDR-H3 of SEQ ID NO: 42, and a light chain variable domain comprising the CDR-L1 of SEQ ID NO: 58, the CDR-L2 of SEQ ID NO: 74, and the CDR-L3 of SEQ ID NO: 90;
(xi) a heavy chain variable domain comprising the CDR-H1 of SEQ ID NO: 11, the CDR-H2 of SEQ ID NO: 27, and the CDR-H3 of SEQ ID NO: 43, and a light chain variable domain comprising the CDR-L1 of SEQ ID NO: 59, the CDR-L2 of SEQ ID NO: 75, and the CDR-L3 of SEQ ID NO: 91;
(xii) a heavy chain variable domain comprising the CDR-H1 of SEQ ID NO: 12, the CDR-H2 of SEQ ID NO: 28, and the CDR-H3 of SEQ ID NO: 44, and a light chain variable domain comprising the CDR-L1 of SEQ ID NO: 60, the CDR-L2 of SEQ ID NO: 76, and the CDR-L3 of SEQ ID NO: 92;
(xiii) a heavy chain variable domain comprising the CDR-H1 of SEQ ID NO: 13, the CDR-H2 of SEQ ID NO: 29, and the CDR-H3 of SEQ ID NO: 45, and a light chain variable domain comprising the CDR-L1 of SEQ ID NO: 61, the CDR-L2 of SEQ ID NO: 77, and the CDR-L3 of SEQ ID NO: 93;
(xiv) a heavy chain variable domain comprising the CDR-H1 of SEQ ID NO: 14, the CDR-H2 of SEQ ID NO: 30, and the CDR-H3 of SEQ ID NO: 46, and a light chain variable domain comprising the CDR-L1 of SEQ ID NO: 62, the CDR-L2 of SEQ ID NO: 78, and the CDR-L3 of SEQ ID NO: 94;
(xv) a heavy chain variable domain comprising the CDR-H1 of SEQ ID NO: 15, the CDR-H2 of SEQ ID NO: 31, and the CDR-H3 of SEQ ID NO: 47, and a light chain variable domain comprising the CDR-L1 of SEQ ID NO: 63, the CDR-L2 of SEQ ID NO: 79, and the CDR-L3 of SEQ ID NO: 95;
(xvi) a heavy chain variable domain comprising the CDR-H1 of SEQ ID NO: 16, the CDR-H2 of SEQ ID NO: 32, and the CDR-H3 of SEQ ID NO: 48, and a light chain variable domain comprising the CDR-L1 of SEQ ID NO: 64, the CDR-L2 of SEQ ID NO: 80, and the CDR-L3 of SEQ ID NO: 96;
(xvii) a heavy chain variable domain comprising the CDR-H1 of SEQ ID NO: 135, the CDR-H2 of SEQ ID NO: 137, and the CDR-H3 of SEQ ID NO: 139, and a light chain variable domain comprising the CDR-L1 of SEQ ID NO: 141, the CDR-L2 of SEQ ID NO: 143, and the CDR-L3 of SEQ ID NO: 145; and
(xviii) a heavy chain variable domain comprising the CDR-H1 of SEQ ID NO: 136, the CDR-H2 of SEQ ID NO: 138, and the CDR-H3 of SEQ ID NO: 140, and a light chain variable domain comprising the CDR-L1 of SEQ ID NO: 142, the CDR-L2 of SEQ ID NO: 144, and the CDR-L3 of SEQ ID NO: 146.

In an embodiment, the MuSK antibody-based molecule comprises:
(ii.a) a heavy chain variable domain comprising the CDR-H1 of SEQ ID NO: 2, the CDR-H2 of SEQ ID NO: 18, and the CDR-H3 of SEQ ID NO: 240, and a light chain variable domain comprising the CDR-L1 of SEQ ID NO: 50, the CDR-L2 of SEQ ID NO: 66, and the CDR-L3 of SEQ ID NO: 82 (X2m1);
(ii.b) a heavy chain variable domain comprising the CDR-H1 of SEQ ID NO: 2, the CDR-H2 of SEQ ID NO: 18, and the CDR-H3 of SEQ ID NO: 241, and a light chain variable domain comprising the CDR-L1 of SEQ ID NO: 50, the CDR-L2 of SEQ ID NO: 66, and the CDR-L3 of SEQ ID NO: 82 (X2m2);
(ii.c) a heavy chain variable domain comprising the CDR-H1 of SEQ ID NO: 2, the CDR-H2 of SEQ ID NO: 18, and the CDR-H3 of SEQ ID NO: 242, and a light chain variable domain comprising the CDR-L1 of SEQ ID NO: 50, the CDR-L2 of SEQ ID NO: 66, and the CDR-L3 of SEQ ID NO: 82 (X2m3);
(ii.d) a heavy chain variable domain comprising the CDR-H1 of SEQ ID NO: 2, the CDR-H2 of SEQ ID NO: 18, and the CDR-H3 of SEQ ID NO: 243, and a light chain variable domain comprising the CDR-L1 of SEQ ID NO: 50, the CDR-L2 of SEQ ID NO: 66, and the CDR-L3 of SEQ ID NO: 82 (X2m4);
(ii.e) a heavy chain variable domain comprising the CDR-H1 of SEQ ID NO: 2, the CDR-H2 of SEQ ID NO: 18, and the CDR-H3 of SEQ ID NO: 244, and a light chain variable domain comprising the CDR-L1 of SEQ ID NO: 50, the CDR-L2 of SEQ ID NO: 66, and the CDR-L3 of SEQ ID NO: 82 (X2m5);
(ii.f) a heavy chain variable domain comprising the CDR-H1 of SEQ ID NO: 2, the CDR-H2 of SEQ ID NO: 18, and the CDR-H3 of SEQ ID NO: 245, and a light chain variable domain comprising the CDR-L1 of SEQ ID NO: 50, the CDR-L2 of SEQ ID NO: 66, and the CDR-L3 of SEQ ID NO: 82 (X2m6);
(ii.g) a heavy chain variable domain comprising the CDR-H1 of SEQ ID NO: 2, the CDR-H2 of SEQ ID NO: 18, and the CDR-H3 of SEQ ID NO: 246, and a light chain variable domain comprising the CDR-L1 of SEQ ID NO: 50, the CDR-L2 of SEQ ID NO: 66, and the CDR-L3 of SEQ ID NO: 82 (X2m7);
(ii.f) a heavy chain variable domain comprising the CDR-H1 of SEQ ID NO: 2, the CDR-H2 of SEQ ID NO: 18, and the CDR-H3 of SEQ ID NO: 247, and a light chain variable domain comprising the CDR-L1 of SEQ ID NO: 50, the CDR-L2 of SEQ ID NO: 66, and the CDR-L3 of SEQ ID NO: 82 (X2m8).

In an embodiment, the MuSK antibody-based molecule comprises:
(xvii.a) a heavy chain variable domain comprising the CDR-H1 of SEQ ID NO: 135, the CDR-H2 of SEQ ID NO: 137, and the CDR-H3 of SEQ ID NO: 248, and a light chain variable domain comprising the CDR-L1 of SEQ ID NO: 141, the CDR-L2 of SEQ ID NO: 143, and the CDR-L3 of SEQ ID NO: 145 (X17m1);
(xvii.b) a heavy chain variable domain comprising the CDR-H1 of SEQ ID NO: 135, the CDR-H2 of SEQ ID NO: 137, and the CDR-H3 of SEQ ID NO: 249, and a light chain variable domain comprising the CDR-L1 of SEQ ID NO: 141, the CDR-L2 of SEQ ID NO: 143, and the CDR-L3 of SEQ ID NO: 145 (X17m2);
(xvii.c) a heavy chain variable domain comprising the CDR-H1 of SEQ ID NO: 135, the CDR-H2 of SEQ ID NO: 137, and the CDR-H3 of SEQ ID NO: 250, and a light chain variable domain comprising the CDR-L1 of SEQ ID NO: 141, the CDR-L2 of SEQ ID NO: 143, and the CDR-L3 of SEQ ID NO: 145 (X17m3);
(xvii.d) a heavy chain variable domain comprising the CDR-H1 of SEQ ID NO: 135, the CDR-H2 of SEQ ID NO: 137, and the CDR-H3 of SEQ ID NO: 251, and a light chain variable domain comprising the CDR-L1 of SEQ ID NO: 141, the CDR-L2 of SEQ ID NO: 143, and the CDR-L3 of SEQ ID NO: 145 (X17m6).

In an embodiment, the MuSK antibody-based molecule comprises:
(i) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 170, and the CDR-L3 of SEQ ID NO: 180 (14D10);
(ii) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 171, and the CDR-L3 of SEQ ID NO: 181 (7G4);
(iii) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 160, the CDR-L2 of SEQ ID NO: 172, and the CDR-L3 of SEQ ID NO: 182 (3C4);
(iv) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2);
(v) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 171, and the CDR-L3 of SEQ ID NO: 184 (3G3);
(vi) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 173, and the CDR-L3 of SEQ ID NO: 185 (31G2);
(vii) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 173, and the CDR-L3 of SEQ ID NO: 186 (31B7);
(viii) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 161, the CDR-L2 of SEQ ID NO: 174, and the CDR-L3 of SEQ ID NO: 187 (17H10);
(ix) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 162, the CDR-L2 of SEQ ID NO: 174, and the CDR-L3 of SEQ ID NO: 188 (23B6);
(x) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 163, the CDR-L2 of SEQ ID NO: 174, and the CDR-L3 of SEQ ID NO: 188 (30E1);
(xi) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 164, the CDR-L2 of SEQ ID NO: 174, and the CDR-L3 of SEQ ID NO: 189 (30A11);
(xii) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 165, the CDR-L2 of SEQ ID NO: 175, and the CDR-L3 of SEQ ID NO: 190 (16F11);
(xiii) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 166, the CDR-L2 of SEQ ID NO: 176, and the CDR-L3 of SEQ ID NO: 191 (4C11);
(xiv) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 167, the CDR-L2 of SEQ ID NO: 177, and the CDR-L3 of SEQ ID NO: 192 (7A12);
(xv) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 168, the CDR-L2 of SEQ ID NO: 178, and the CDR-L3 of SEQ ID NO: 193 (7G12);
(xvi) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 169, the CDR-L2 of SEQ ID NO: 179, and the CDR-L3 of SEQ ID NO: 194 (7B8);
(xvii) a heavy chain variable domain comprising the CDR-H1 of SEQ ID NO: 147, the CDR-H2 of SEQ ID NO: 153, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1 m1);
(xviii) a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1 m2);
(xvix) a heavy chain variable domain comprising the CDR-H1 of SEQ ID NO: 147, the CDR-H2 of SEQ ID NO: 155, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m4);
(xx) a heavy chain variable domain comprising the CDR-H1 of SEQ ID NO: 147, the CDR-H2 of SEQ ID NO: 153, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m1);
(xxi) a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m2); and
(xxii) a heavy chain variable domain comprising the CDR-H1 of SEQ ID NO: 147, the CDR-H2 of SEQ ID NO: 155, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m4)

In a preferred embodiment, the antibody-based molecule that binds to human muscle-specific tyrosine-protein kinase (MuSK) comprises a heavy chain variable domain and a light chain variable domain, where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO:156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the CDR-H2 of the antibody comprises a proline (P) at position 3, a tryptophan (W) at position 4, and a serine (S) or asparagine (N) at position 5.

In a more preferred embodiment, the antibody-based molecule that binds to human muscle-specific tyrosine-protein kinase (MuSK) comprises a heavy chain variable domain and a light chain variable domain,

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

The MuSK antibody-based molecule as described herein may comprise a variable light (VL) chain, a variable heavy (VH) chain, or a combination of VL and VH chains. In some embodiments, the VH chain of the MuSK antibody-based molecule comprises any one of the VH amino acid sequences provided in Table 3 below, or an amino acid sequence that is at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, identical or similar to any one of the VH amino acid sequences listed in Table 3. In some embodiments, the VL chain of the MuSK antibody-based molecule comprises any one of the VL amino acid sequences provided in Table 3 below, or an amino acid sequence that is at least 60%, identical or similar to any one of the VL amino acid sequences listed in Table 3. In an embodiment, the identity or similarity is at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%.

| mAb/Fab | | | SEQ ID |
|---|---|---|---|
| name | Domain | Sequence | NO: |
| X1 | VH | EVQLVESGGGLVQPGGSLRLSCAASGFTFSSSSIHWRQAPGKGLEWVA | 97 |
| | | SISSSSGSTSYADSVKGRFTISADTSKNTAYLQMNSLRAEDTAVYYCARKY | |
| | | WSQYYWAHYYGGLDYWGQGTLVTVSS | |
| | VL | DIQMTQSPSSLSASVGDRVTITCRASQSVSSAVAWYQQKPGKAPKLLIYSA | 98 |
| | | SSLYSGVPSRFSGSRSGTDFTLTISSLQPEDFATYYCQQSSSSLITFGQGT | |
| | | KVEIK | |
| X2 | VH | EVQLVESGGGLVQPGGSLRLSCAASGFTVSSSSIHWVRQAPGKGLEWVA | 99 |
| | | SISSSYGSTSYADSVKGRFTISADTSKNTAYLQMNSLRAEDTAVYYCARSE | |
| | | GDRYVSGYMGMDYWGQGTLVTVSS | |
| X2m1 | VH | EVQLVESGGGLVQPGGSLRLSCAASGFTVSSSSIHWVRQAPGKGLEWVA | 252 |
| | | SISSSYGSTSYADSVKGRFTISADTSKNTAYLQMNSLRAEDTAVYYCARSE | |
| | | GDRYVSGYFGFDYWGQGTLVTVSS | |
| X2m2 | VH | EVQLVESGGGLVQPGGSLRLSCAASGFTVSSSSIHWVRQAPGKGLEWVA | 253 |
| | | SISSSYGSTSYADSVKGRFTISADTSKNTAYLQMNSLRAEDTAVYYCARSE | |
| | | GDRYVSGYFGLDYWGQGTLVTVSS | |
| X2m3 | VH | EVQLVESGGGLVQPGGSLRLSCAASGFTVSSSSIHWVRQAPGKGLEWVA | 254 |
| | | SISSSYGSTSYADSVKGRFTISADTSKNTAYLQMNSLRAEDTAVYYCARSE | |
| | | GDRYVSGYSGFDYWGQGTLVTVSS | |
| X2m4 | VH | EVQLVESGGGLVQPGGSLRLSCAASGFTVSSSSIHWVRQAPGKGLEWVA | 255 |
| | | SISSSYGSTSYADSVKGRFTISADTSKNTAYLQMNSLRAEDTAVYYCARSE | |
| | | GDRYVSGYSGLDYWGQGTLVTVSS | |
| X2m5 | VH | EVQLVESGGGLVQPGGSLRLSCAASGFTVSSSSIHWVRQAPGKGLEWVA | 256 |
| | | SISSSYGSTSYADSVKGRFTISADTSKNTAYLQMNSLRAEDTAVYYCARSE | |
| | | GDRYVSGYFGMDYWGQGTLVTVSS | |
| X2m6 | VH | EVQLVESGGGLVQPGGSLRLSCAASGFTVSSSSIHWVRQAPGKGLEWVA | 257 |
| | | SISSSYGSTSYADSVKGRFTISADTSKNTAYLQMNSLRAEDTAVYYCARSE | |
| | | GDRYVSGYSGMDYWGQGTLVTVSS | |
| X2m7 | VH | EVQLVESGGGLVQPGGSLRLSCAASGFTVSSSSIHWVRQAPGKGLEWVA | 258 |
| | | SISSSYGSTSYADSVKGRFTISADTSKNTAYLQMNSLRAEDTAVYYCARSE | |
| | | GDRYVSGYMGFDYWGQGTLVTVSS | |
| X2m8 | VH | EVQLVESGGGLVQPGGSLRLSCAASGFTVSSSSIHWVRQAPGKGLEWVA | 259 |
| | | SISSSYGSTSYADSVKGRFTISADTSKNTAYLQMNSLRAEDTAVYYCARSE | |
| | | GDRYVSGYMGLDYWGQGTLVTVSS | |
| X2 | VL | DIQMTQSPSSLSASVGDRVTITCRASQSVSSAVAWYQQKPGKAPKLLIYSA | 100 |
| | | SSLYSGVPSRFSGSRSGTDFTLTISSLQPEDFATYYCQQSGVWLITFGQGT | |
| | | KVEIK | |
| X3 | VH | EVQLVESGGGLVQPGGSLRLSCAASGFTISSSSIHWVRQAPGKGLEWVAS | 101 |
| | | ISSSSGYTYYADSVKGRFTISADTSKNTAYLQMNSLRAEDTAVYYCARSWY | |
| | | EMWMSGYFGFDYWGQGTLVTVSS | |
| | VL | DIQMTQSPSSLSASVGDRVTITCRASQSVSSAVAWYQQKPGKAPKLLIYSA | 102 |
| | | SSLYSGVPSRFSGSRSGTDFTLTISSLQPEDFATYYCQQSSSSLITFGQGT | |
| | | KVEIK | |
| X4 | VH | EVQLVESGGGLVQPGGSLRLSCAASGFTVSSSSIHWVRQAPGKGLEWVA | 103 |
| | | SISSSSGSTYYADSVKGRFTISADTSKNTAYLQMNSLRAEDTAVYYCARGE | |
| | | HDYYVFGYLGMDYWGQGTLVTVSS | |
| | VL | DIQMTQSPSSLSASVGDRVTITCRASQSVSSAVAWYQQKPGKAPKLLIYSA | 104 |
| | | SSLYSGVPSRFSGSRSGTDFTLTISSLQPEDFATYYCQQSYKPGALITFGQ | |
| | | GTKVEIK | |
| X5 | VH | EVQLVESGGGLVQPGGSLRLSCAASGFTFYSSSIHWVRQAPGKGLEWVA | 105 |
| | | SISSSSGSTSYADSVKGRFTISADTSKNTAYLQMNSLRAEDTAVYYCARSY | |
| | | TMFYYGGWYGSGYFGMDYWGQGTLVTVSS | |
| | VL | DIQMTQSPSSLSASVGDRVTITCRASQSVSSAVAWYQQKPGKAPKLLIYSA | 106 |
| | | SSLYSGVPSRFSGSRSGTDFTLTISSLQPEDFATYYCQQSSSSLITFGQGT | |
| | | KVEIK | |
| X6 | VH | EVQLVESGGGLVQPGGSLRLSCAASGFTFSSSSIHWVRQAPGKGLEWVA | 107 |
| | | SISSYSGYTYYADSVKGRFTISADTSKNTAYLQMNSLRAEDTAVYYCARTY | |
| | | GSYYVSSYTGMDYWGQGTLVTVSS | |
| | VL | DIQMTQSPSSLSASVGDRVTITCRASQSVSSAVAWYQQKPGKAPKLLIYSA | 108 |
| | | SSLYSGVPSRFSGSRSGTDFTLTISSLQPEDFATYYCQQSSSSLITFGQGT | |
| | | KVEIK | |
| X7 | VH | EVQLVESGGGLVQPGGSLRLSCAASGFTLYSSSIHWVRQAPGKGLEWVA | 109 |
| | | SISSSYSSTYYADSVKGRFTISADTSKNTAYLQMNSLRAEDTAVYYCARLA | |
| | | GLYHYPGYLGLDYWGQGTLVTVSS | |
| | VL | DIQMTQSPSSLSASVGDRVTITCRASQSVSSAVAWYQQKPGKAPKLLIYSA | 110 |
| | | SSLYSGVPSRFSGSRSGTDFTLTISSLQPEDFATYYCQQSSRSSLLTFGQG | |
| | | TKVEIK | |
| X8 | VH | EVQLVESGGGLVQPGGSLRLSCAASGFTVSSSSIHWVRQAPGKGLEWVA | 111 |
| | | SISSSSGSTSYADSVKGRFTISADTSKNTAYLQMNSLRAEDTAVYYCARSW | |
| | | SYHPWYYHVGWYTGLDYWGQGTLVTVSS | |
| | VL | DIQMTQSPSSLSASVGDRVTITCRASQSVSSAVAWYQQKPGKAPKLLIYSA | 112 |
| | | SSLYSGVPSRFSGSRSGTDFTLTISSLQPEDFATYYCQQSSSSLITFGQGT | |
| | | KVEIK | |
| X9 | VH | EVQLVESGGGLVQPGGSLRLSCAASGFTFSSSSIHWVRQAPGKGLEWVA | 113 |
| | | SIYSSSGSTYYADSVKGRFTISADTSKNTAYLQMNSLRAEDTAVYYCARSG | |
| | | GEFYITSYYGMDYWGQGTLVTVSS | |
| | VL | DIQMTQSPSSLSASVGDRVTITCRASQSVSSAVAWYQQKPGKAPKLLIYSA | 114 |
| | | SSLYSGVPSRFSGSRSGTDFTLTISSLQPEDFATYYCQQSSSSLITFGQGT | |
| | | KVEIK | |
| X10 | VH | EVQLVESGGGLVQPGGSLRLSCAASGFTFSSSSIHWVRQAPGKGLEWVA | 115 |
| | | SISSSYSSTSYADSVKGRFTISADTSKNTAYLQMNSLRAEDTAVYYCARKY | |
| | | YRWRHNKYQGFDYWGQGTLVTVSS | |
| | VL | DIQMTQSPSSLSASVGDRVTITCRASQSVSSAVAWYQQKPGKAPKLLIYSA | 116 |
| | | SSLYSGVPSRFSGSRSGTDFTLTISSLQPEDFATYYCQQSLWYPVTFGQG | |
| | | TKVEIK | |
| X11 | VH | EVQLVESGGGLVQPGGSLRLSCAASGFTISSSSIHWVRQAPGKGLEWVAS | 117 |
| | | ISSYSGSTYYADSVKGRFTISADTSKNTAYLQMNSLRAEDTAVYYCARSWG | |
| | | SYYVSGFVGFDYWGQGTLVTVSS | |
| | VL | DIQMTQSPSSLSASVGDRVTITCRASQSVSSAVAWYQQKPGKAPKLLIYSA | 118 |
| | | SSLYSGVPSRFSGSRSGTDFTLTISSLQPEDFATYYCQQNSYYLITFGQGT | |
| | | KVEIK | |
| X12 | VH | EVQLVESGGGLVQPGGSLRLSCAASGFTISSSSIHWVRQAPGKGLEWVAY | 119 |
| | | ISPSSGYTSYADSVKGRFTISADTSKNTAYLQMNSLRAEDTAVYYCARQYW | |
| | | VPQWWITQYFGMDYWGQGTLVTVSS | |
| | VL | DIQMTQSPSSLSASVGDRVTITCRASQSVSSAVAWYQQKPGKAPKLLIYSA | 120 |
| | | SSLYSGVPSRFSGSRSGTDFTLTISSLQPEDFATYYCQQSSSSLITFGQGT | |
| | | KVEIK | |
| X13 | VH | EVQLVESGGGLVQPGGSLRLSCAASGFTISSSSIHWVRQAPGKGLEWVAS | 121 |
| | | ISSSSGSTSYADSVKGRFTISADTSKNTAYLQMNSLRAEDTAVYYCARSSE | |
| | | HWYTIGYYGIDYWGQGTLVTVSS | |
| | VL | DIQMTQSPSSLSASVGDRVTITCRASQSVSSAVAWYQQKPGKAPKLLIYSA | 122 |
| | | SSLYSGVPSRFSGSRSGTDFTLTISSLQPEDFATYYCQQSYGSFSLITFGQ | |
| | | GTKVEIK | |
| X14 | VH | EVQLVESGGGLVQPGGSLRLSCAASGFTFSSSSIHWVRQAPGKGLEWVA | 123 |
| | | SISSSSGYTYYADSVKGRFTISADTSKNTAYLQMNSLRAEDTAVYYCARGS | |
| | | HHWFLWIYSGLDYWGQGTLVTVSS | |
| | VL | DIQMTQSPSSLSASVGDRVTITCRASQSVSSAVAWYQQKPGKAPKLLIYSA | 124 |
| | | SSLYSGVPSRFSGSRSGTDFTLTISSLQPEDFATYYCQQGSYHLITFGQGT | |
| | | KVEIK | |
| X15 | VH | EVQLVESGGGLVQPGGSLRLSCAASGFTVSSSSIHWVRQAPGKGLEWVA | 125 |
| | | SISSSYGSTSYADSVKGRFTISADTSKNTAYLQMNSLRAEDTAVYYCARSE | |
| | | GDRYVSGYMGMDYWGQGTLVTVSS | |
| | VL | DIQMTQSPSSLSASVGDRVTITCRASQSVSSAVAWYQQKPGKAPKLLIYSA | 126 |
| | | SSLYSGVPSRFSGSRSGTDFTLTISSLQPEDFATYYCQQSGVWLITFGQGT | |
| | | KVEIK | |
| X16 | VH | EVQLVESGGGLVQPGGSLRLSCAASGFTFSSSSIHWVRQAPGKGLEWVA | 127 |
| | | SIYSSYGYTSYADSVKGRFTISADTSKNTAYLQMNSLRAEDTAVYYCARNW | |
| | | GYYMYWGWYYALDYWGQGTLVTVSS | |
| | VL | DIQMTQSPSSLSASVGDRVTITCRASQSVSSAVAWYQQKPGKAPKLLIYSA | 128 |
| | | SSLYSGVPSRFSGSRSGTDFTLTISSLQPEDFATYYCQQWSSAQALITFGQ | |
| | | GTKVEIK | |
| X17 | VH | EVQLVESGGGLVQPGGSLRLSCAASGFTISYSSIHWVRQAPGKGLEWVAS | 131 |
| | | IYSSSGSTYYADSVKGRFTISADTSKNTAYLQMNSLRAEDTAVYYCARGDH | |
| | | GYYVFGYLGMDYWGQGTLVTVSS | |
| X17m1 | VH | EVQLVESGGGLVQPGGSLRLSCAASGFTISYSSIHWVRQAPGKGLEWVAS | 260 |
| | | IYSSSGSTYYADSVKGRFTISADTSKNTAYLQMNSLRAEDTAVYYCARGDH | |
| | | GYYVSGYLGMDYWGQGTLVTVSS | |
| X17m2 | VH | EVQLVESGGGLVQPGGSLRLSCAASGFTISYSSIHWVRQAPGKGLEWVAS | 261 |
| | | IYSSSGSTYYADSVKGRFTISADTSKNTAYLQMNSLRAEDTAVYYCARGDH | |
| | | GYYVYGYLGMDYWGQGTLVTVSS | |
| X17m3 | VH | EVQLVESGGGLVQPGGSLRLSCAASGFTISYSSIHWVRQAPGKGLEWVAS | 262 |
| | | IYSSSGSTYYADSVKGRFTISADTSKNTAYLQMNSLRAEDTAVYYCARGDH | |
| | | GYYVSGYLGFDYWGQGTLVTVSS | |
| X17m6 | VH | EVQLVESGGGLVQPGGSLRLSCAASGFTISYSSIHWVRQAPGKGLEWVAS | 263 |
| | | IYSSSGSTYYADSVKGRFTISADTSKNTAYLQMNSLRAEDTAVYYCARGEH | |
| | | GYYVSGYLGFDYWGQGTLVTVSS | |
| X17 | VL | DIQMTQSPSSLSASVGDRVTITCRASQSVSSAVAWYQQKPGKAPKLLIYSA | 132 |
| | | SSLYSGVPSRFSGSRSGTDFTLTISSLQPEDFATYYCQQSYKPGALITFGQ | |
| | | GTKVEIK | |
| X18 | VH | EVQLVESGGGLVQPGGSLRLSCAASGFTISSSSIHWVRQAPGKGLEWVAS | 133 |
| | | ISSSSGYTSYADSVKGRFTISADTSKNTAYLQMNSLRAEDTAVYYCARKYS | |
| | | KRAYPDYYWRGLDYWGQGTLVTVSS | |
| | VL | DIQMTQSPSSLSASVGDRVTITCRASQSVSSAVAWYQQKPGKAPKLLIYSA | 134 |
| | | SSLYSGVPSRFSGSRSGTDFTLTISSLQPEDFATYYCQQSYWWPITFGQGT | |
| | | KVEIK | |
| 14D10 | VH | ELQLVESGGGLVQPGGSLRLSCAASGFTFDDYGMSWVRQAPGKGLEWV | 196 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRTLI | 197 |
| | | YTTNSRHSGVPSRFSGSISGNKAALTITGAQPEDEADYYCALYMGGGSNV | |
| | | YVFGGGTKLTVL | |
| 7G4 | VH | ELQLVESGGGLVQPGGSLRLSCAASGFTFDDYGMSWVRQAPGKGLEWV | 198 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRALI | 199 |
| | | YSTNSRHSGVPSRFSGSISGNKAALTITGAQPEDEADYYCALYMGRGSNK | |
| | | DYVFGGGTKLTVL | |
| 3C4 | VH | ELQLVESGGGLVQPGGSLRLSCAASGFTFDDYGMSWVRQAPGKGLEWV | 200 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTASNYPDWYQQTPGQAPRGLI | 201 |
| | | YSTDSRHSGVPSRFSGSISGNKAALTITGAQPEDEADYYCALYMYSDSKLY | |
| | | VFGGGTKLTVL | |
| 3B2 | VH | ELQLVESGGGLVQPGGSLRLSCAASGFTFDDYGMSWVRQAPGKGLEWV | 202 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRGLI | 203 |
| | | YSTDSRHSGVPSRFSGSISGNKAALTITGAQSEDEADYYCGLYMYSGSKN | |
| | | YVFGGGTKLTVL | |
| 3G3 | VH | ELQLVESGGGLVQPGGSLRLSCAASGFTFDDYGMSWVRQAPGKGLEWV | 204 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QTVVTQEPSLSVSPGGTVTLTCGLSSGSVTSSNYPDWYQQTPGQAPRALI | 205 |
| | | YSTNSRHSGVPSRFSGSTSGNKAALTITGAQPEDEADYYCALYMGSDIRN | |
| | | YVFGGGTKLTVL | |
| 31G2 | VH | ELQLVESGGGLVQPGGSLRLSCAASGFTFDDYGMSWVRQAPGKGLEWV | 206 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRALI | 207 |
| | | YSTNSRLSGVPSRFSGSFSGNKAALTITGAQPEDEADYYCALYMGSGSRN | |
| | | YVFGGGTKLTVL | |
| 31B7 | VH | ELQLVESGGGLVQPGGSLRLSCAASGFTFDDYGMSWVRQAPGKGLEWV | 208 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRALI | 209 |
| | | YSTNSRLSGVPSRFSGSFSGNKAALTITGAQPEDEADYYCALYMGSESRN | |
| | | YVFGGGTKLTVL | |
| 17H10 | VH | QVQVQESGPGLVKPSQTLSLTCTVSGGSITARYYSWSWIRQPPGKGLEW | 210 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVSVALRQTAKITCGGNRIGGKSVQWYQQKPGQAPMLVIYAD | 211 |
| | | SRRPSGIPERFTGSNSGNTATLTITGAQAEDEADYYCHVWGSTASADFGG | |
| | | GTHLTVL | |
| 23B6 | VH | QVQVQESGPGLVKPSQTLSLTCTVSGGSITARYYSWSWIRQPPGKGLEW | 212 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVSVALRQTAKITCGGDNIGSKNAQWYQQKPGQAPVMVLYAD | 213 |
| | | SRRPSGIPERFSGSNSGNTATLTISGAQAEDEADYYCHVWDSSTNAWFGG | |
| | | GTHLTVL | |
| 30E1 | VH | QVQVQESGPGLVKPSQTLSLTCTVSGGSITARYYSWSWIRQPPGKGLEW | 214 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVSVALRRTAKITCGGDNIGSKNTQWYQQKPGQAPVLVIYADS | 215 |
| | | RRPSGIPERFSGSNSGNTATLTISGAQAEDEADYYCHVWDSSTNAWFGGG | |
| | | THLTVL | |
| 30A11 | VH | QVQVQESGPGLVKPSQTLSLTCTVSGGSITARYYSWSWIRQPPGKGLEW | 216 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVTVALRQTAKITCGGDNIASKNVQWYQQKPGQAPSLVIWAD | 217 |
| | | SRRPSGIPVRFSGSNFGNTATLTISGAQAEDEADYYCQVWDSSTNVAVFG | |
| | | GGTHLTVL | |
| 16F11 | VH | EVQLVESGGGLVQPGGSLSLSCVASGFTFSLYYMNWVRQAPGKGLEWLS | 218 |
| | | VIDTHSIAYYADSVKGRFTISRDNVKNTLYLQLNNLKPEDTALYYCVLGRTAL | |
| | | VRWGQGTQVTVSS | |
| | VL | DIVMTQSPSSVTASVGEKVTINCKSSQSVVFGSNQKSYLNWYQQRPGQSP | 219 |
| | | RLLIYYASTQESGIPDRFSGSGSTTDFTLTISSVQPEDAAVYYCQQAYSAPT | |
| | | FGSGTRLEIK | |
| 4C11 | VH | EVQLVESGGGLVQPGGSLSLSCVASGFTFSLYYMNWVRQAPGKGLEWLS | 220 |
| | | VIDTHSIAYYADSVKGRFTISRDNVKNTLYLQLNNLKPEDTALYYCVLGRTAL | |
| | | VRWGQGTQVTVSS | |
| | VL | DIVMTQSPSSVTASAGERVTINCRSSQSVLYSSNQKNYLNWYQQRLGQSP | 221 |
| | | RLLIYWASARESGVPDRFSGSGSTTNFTLTISSFQPEDAAVYYCQQSYKPP | |
| | | YGFGSGTRLEIK | |
| 7A12 | VH | EVQLVESGGGLVQPGGSLSLSCVASGFTFSLYYMNWVRQAPGKGLEWLS | 222 |
| | | VIDTHSIAYYADSVKGRFTISRDNVKNTLYLQLNNLKPEDTALYYCVLGRTAL | |
| | | VRWGQGTQVTVSS | |
| | VL | EIVLTQSPSSVTASIGEKVTINCESSQSVLYNQKNYLNWYQQRPGQSPRLLI | 223 |
| | | YWASTRQSGVPDRFSGSGSGSTTDFTLTISSFQPEDVAVYYCQQAYNAPL | |
| | | TFGPGTKVELK | |
| 7G12 | VH | EVQLVESGGGLVQPGGSLSLSCVASGFTFSLYYMNWVRQAPGKGLEWLS | 224 |
| | | VIDTHSIAYYADSVKGRFTISRDNVKNTLYLQLNNLKPEDTALYYCVLGRTAL | |
| | | VRWGQGTQVTVSS | |
| | VL | EIVLTQSPNSVTASVGEKVTINCKSSQRVQLGSNQKSYLNWYQQRPGQSP | 225 |
| | | RLLIYYASTQQSGIPDRFSGSGSATDFTLTINSVQPEDAAVYYCQQGYSAP | |
| | | FTFGQGTKVELK | |
| 7B8 | VH | EVQLVESGGGLVQPGGSLSLSCVASGFTFSLYYMNWVRQAPGKGLEWLS | 226 |
| | | VIDTHSIAYYADSVKGRFTISRDNVKNTLYLQLNNLKPEDTALYYCVLGRTAL | |
| | | VRWGQGTQVTVSS | |
| | VL | EIVLTQSPSSVTASAGEKVTINCKSSQSVLYNQKNYLAWYQQRPGQSPRLL | 227 |
| | | IYWASTRESGVPDRFSGSGSTTDFTLTISSFQPEDVAVYYCQQGYSVPYTF | |
| | | GSGTRLEIK | |
| 3B2g1m1 | VH | EVQLLESGGGLVQPGGSLRLSCAASGFTFSDYGMSWVRQAPGKGLEWVS | 228 |
| | | AIPWSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 229 |
| | | YSTDSRHSGVPDRFSGSILGNKAALTITGAQADDESDYYCGLYMYSGSKN | |
| | | YVFGGGTKLTVL | |
| 3B2g1m2 | VH | EVQLLESGGGLVQPGGSLRLSCAASGFTFSDYGMSWVRQAPGKGLEWVS | 230 |
| | | AIPGSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 231 |
| | | YSTDSRHSGVPDRFSGSILGNKAALTITGAQADDESDYYCGLYMYSGSKN | |
| | | YVFGGGTKLTVL | |
| 3B2g1m4 | VH | EVQLLESGGGLVQPGGSLRLSCAASGFTFSDYGMSWVRQAPGKGLEWVS | 232 |
| | | AIPWQGGSTYYKESVKGRFTISRDNSKNTLYLQMNSLRAEDTAVYYCAKR | |
| | | SGRIAFGALDAWGQGTLVTVSS | |
| | VL | QTVVTQEPSFSVSPGGTVTLTCGLSSGSVTSSNYPDWYQQTPGQAPRTLI | 233 |
| | | YSTDSRHSGVPDRFSGSILGNKAALTITGAQADDESDYYCGLYMYSGSKN | |
| | | YVFGGGTKLTVL | |
| 3B2g2m1 | VH | EVQLLESGGGLVQPGGSLRLSCAASGFTFSDYGMSWVRQAPGKGLEWVS | 234 |
| | | AIPWSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 235 |
| | | YSTDSRHSGVPDRFSGSILGNKAALTITGAQADDESDYYCGLYSYSGSKNY | |
| | | VFGGGTKLTVL | |
| 3B2g2m2 | VH | EVQLLESGGGLVQPGGSLRLSCAASGFTFSDYGMSWVRQAPGKGLEWVS | 236 |
| | | AIPGSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 237 |
| | | YSTDSRHSGVPDRFSGSILGNKAALTITGAQADDESDYYCGLYSYSGSKNY | |
| | | VFGGGTKLTVL | |
| 3B2g2m4 | VH | EVQLLESGGGLVQPGGSLRLSCAASGFTFSDYGMSWVRQAPGKGLEWVS | 238 |
| | | AIPWQGGSTYYKESVKGRFTISRDNSKNTLYLQMNSLRAEDTAVYYCAKR | |
| | | SGRIAFGALDAWGQGTLVTVSS | |
| | VL | QTVVTQEPSFSVSPGGTVTLTCGLSSGSVTSSNYPDWYQQTPGQAPRTLI | 239 |
| | | YSTDSRHSGVPDRFSGSILGNKAALTITGAQADDESDYYCGLYSYSGSKNY | |
| | | VFGGGTKLTVL | |

In an embodiment, the MuSK antibody-based molecule disclosed herein comprises:
a heavy chain variable domain comprising an amino acid sequence that is at least 80% identical to SEQ ID NO: 97 and a light chain variable domain comprising an amino acid sequence that is at least 80% identical to SEQ ID NO: 98; (ii) a heavy chain variable domain comprising an amino acid sequence that is at least 80% identical to any one of SEQ ID NOs: 99 and 252-259 and a light chain variable domain comprising an amino acid sequence that is at least 80% identical to SEQ ID NO: 100; (iii) a heavy chain variable domain comprising an amino acid sequence that is at least 80% identical to SEQ ID NO: 101 and a light chain variable domain comprising an amino acid sequence that is at least 80% identical to SEQ ID NO: 102; (iv) a heavy chain variable domain comprising an amino acid sequence that is at least 80% identical to SEQ ID NO: 103 and a light chain variable domain comprising an amino acid sequence that is at least 80% identical to SEQ ID NO: 104; (v) a heavy chain variable domain comprising an amino acid sequence that is at least 80% identical to SEQ ID NO: 105 and a light chain variable domain comprising an amino acid sequence that is at least 80% identical to SEQ ID NO: 106; (vi) a heavy chain variable domain comprising an amino acid sequence that is at least 80% identical to SEQ ID NO: 107 and a light chain variable domain comprising an amino acid sequence that is at least 80% identical to SEQ ID NO: 108; or (vii) a heavy chain variable domain comprising an amino acid sequence that is at least 80% identical to SEQ ID NO: 109 and a light chain variable domain comprising an amino acid sequence that is at least 80% identical to SEQ ID NO: 110; (viii) a heavy chain variable domain comprising an amino acid sequence that is at least 80% identical to SEQ ID NO: 111 and a light chain variable domain comprising an amino acid sequence that is at least 80% identical to SEQ ID NO: 112; (ix) a heavy chain variable domain comprising an amino acid sequence that is at least 80% identical to SEQ ID NO: 113 and a light chain variable domain comprising an amino acid sequence that is at least 80% identical to SEQ ID NO: 114; (x) a heavy chain variable domain comprising an amino acid sequence that is at least 80% identical to SEQ ID NO: 115 and a light chain variable domain comprising an amino acid sequence that is at least 80% identical to SEQ ID NO: 116; (xi) a heavy chain variable domain comprising an amino acid sequence that is at least 80% identical to SEQ ID NO: 117 and a light chain variable domain comprising an amino acid sequence that is at least 80% identical to SEQ ID NO: 118; (xii) a heavy chain variable domain comprising an amino acid sequence that is at least 80% identical to SEQ ID NO: 119 and a light chain variable domain comprising an amino acid sequence that is at least 80% identical to SEQ ID NO: 120; (xiii) a heavy chain variable domain comprising an amino acid sequence that is at least 80% identical to SEQ ID NO: 121 and a light chain variable domain comprising an amino acid sequence that is at least 80% identical to SEQ ID NO: 122; (xiv) a heavy chain variable domain comprising an amino acid sequence that is at least 80% identical to SEQ ID NO: 123 and a light chain variable domain comprising an amino acid sequence that is at least 80% identical to SEQ ID NO: 124; (xv) a heavy chain variable domain comprising an amino acid sequence that is at least 80% identical to SEQ ID NO: 125 and a light chain variable domain comprising an amino acid sequence that is at least 80% identical to SEQ ID NO: 126; (xvi) a heavy chain variable domain comprising an amino acid sequence that is at least 80% identical to SEQ ID NO: 127 and a light chain variable domain comprising an amino acid sequence that is at least 80% identical to SEQ ID NO: 128; (xvii) a heavy chain variable domain comprising an amino acid sequence that is at least 80% identical to any one of SEQ ID NOs: 131 and 260-263 and a light chain variable domain comprising an amino acid sequence that is at least 80% identical to SEQ ID NO: 132; and (xviii) a heavy chain variable domain comprising an amino acid sequence that is at least 80% identical to SEQ ID NO: 133 and a light chain variable domain comprising an amino acid sequence that is at least 80% identical to SEQ ID NO: 134.

In some embodiments, the MuSK antibody-based molecule disclosed herein comprises: (i) a heavy chain variable domain comprising an amino acid sequence that is at least 80% identical to SEQ ID NO: 196 and a light chain variable domain comprising an amino acid sequence that is at least 80% identical to SEQ ID NO: 197; (ii) a heavy chain variable domain comprising an amino acid sequence that is at least 80% identical to SEQ ID NO: 198 and a light chain variable domain comprising an amino acid sequence that is at least 80% identical to SEQ ID NO: 199; (iii) a heavy chain variable domain comprising an amino acid sequence that is at least 80% identical to SEQ ID NO: 200 and a light chain variable domain comprising an amino acid sequence that is at least 80% identical to SEQ ID NO: 201; (iv) a heavy chain variable domain comprising an amino acid sequence that is at least 80% identical to SEQ ID NO: 202 and a light chain variable domain comprising an amino acid sequence that is at least 80% identical to SEQ ID NO: 203; (v) a heavy chain variable domain comprising an amino acid sequence that is at least 80% identical to SEQ ID NO: 204 and a light chain variable domain comprising an amino acid sequence that is at least 80% identical to SEQ ID NO: 205; (vi) a heavy chain variable domain comprising an amino acid sequence that is at least 80% identical to SEQ ID NO: 206 and a light chain variable domain comprising an amino acid sequence that is at least 80% identical to SEQ ID NO: 207; (vii) a heavy chain variable domain comprising an amino acid sequence that is at least 80% identical to SEQ ID NO: 208 and a light chain variable domain comprising an amino acid sequence that is at least 80% identical to SEQ ID NO: 209; (viii) a heavy chain variable domain comprising an amino acid sequence that is at least 80% identical to SEQ ID NO: 210 and a light chain variable domain comprising an amino acid sequence that is at least 80% identical to SEQ ID NO: 211; (vix) a heavy chain variable domain comprising an amino acid sequence that is at least 80% identical to SEQ ID NO: 212 and a light chain variable domain comprising an amino acid sequence that is at least 80% identical to SEQ ID NO: 213; (x) a heavy chain variable domain comprising an amino acid sequence that is at least 80% identical to SEQ ID NO: 214 and a light chain variable domain comprising an amino acid sequence that is at least 80% identical to SEQ ID NO: 215; (xi) a heavy chain variable domain comprising an amino acid sequence that is at least 80% identical to SEQ ID NO: 216 and a light chain variable domain comprising an amino acid sequence that is at least 80% identical to SEQ ID NO: 217; (xii) a heavy chain variable domain comprising an amino acid sequence that is at least 80% identical to SEQ ID NO: 218 and a light chain variable domain comprising an amino acid sequence that is at least 80% identical to SEQ ID NO: 219; (xiii) a heavy chain variable domain comprising an amino acid sequence that is at least 80% identical to SEQ ID NO: 220 and a light chain variable domain comprising an amino acid sequence that is at least 80% identical to SEQ ID NO: 221; (xiv) a heavy chain variable domain comprising an amino acid sequence that is at least 80% identical to SEQ ID NO: 222 and a light chain variable domain comprising an amino acid sequence that is at least 80% identical to SEQ ID NO: 223; (xv) a heavy chain variable domain comprising an amino acid sequence that is at least 80% identical to SEQ ID NO: 224 and a light chain variable domain comprising an amino acid sequence that is at least 80% identical to SEQ ID NO: 225; (xvi) a heavy chain variable domain comprising an amino acid sequence that is at least 80% identical to SEQ ID NO: 226 and a light chain variable domain comprising an amino acid sequence that is at least 80% identical to SEQ ID NO: 227; (xvii) a heavy chain variable domain comprising an amino acid sequence that is at least 80% identical to SEQ ID NO: 228 and a light chain variable domain comprising an amino acid sequence that is at least 80% identical to SEQ ID NO: 229; (xviii) a heavy chain variable domain comprising an amino acid sequence that is at least 80% identical to SEQ ID NO: 230 and a light chain variable domain comprising an amino acid sequence that is at least 80% identical to SEQ ID NO: 231; (xix) a heavy chain variable domain comprising an amino acid sequence that is at least 80% identical to SEQ ID NO: 232 and a light chain variable domain comprising an amino acid sequence that is at least 80% identical to SEQ ID NO: 233; (xx) a heavy chain variable domain comprising an amino acid sequence that is at least 80% identical to SEQ ID NO: 234 and a light chain variable domain comprising an amino acid sequence that is at least 80% identical to SEQ ID NO: 235; (xxi) a heavy chain variable domain comprising an amino acid sequence that is at least 80% identical to SEQ ID NO: 236 and a light chain variable domain comprising an amino acid sequence that is at least 80% identical to SEQ ID NO: 237; (xxii) a heavy chain variable domain comprising an amino acid sequence that is at least 80% identical to SEQ ID NO: 238 and a light chain variable domain comprising an amino acid sequence that is at least 80% identical to SEQ ID NO: 239.

In a preferred embodiment, the MuSK antibody-based molecule (or the anti-MuSK antibody or antigen binding fragment thereof) disclosed herein comprises
a heavy chain variable domain (VH) comprising an amino acid sequence that is at least 80% identical or similar to SEQ ID NO: 234 and a light chain variable domain (VL) comprising an amino acid sequence that is at least 80% identical or similar to SEQ ID NO: 235.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the MuSK antibody-based molecule disclosed herein comprises
a heavy chain variable domain comprising amino acid sequence SEQ ID NO: 234 and a light chain variable domain comprising amino acid sequence SEQ ID NO: 235.

In a preferred embodiment, the antibody-based molecule that binds to human muscle-specific tyrosine-protein kinase (MuSK) comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In a more preferred embodiment, the antibody-based molecule that binds to human muscle-specific tyrosine-protein kinase (MuSK) comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising at least 80% sequence identity to SEQ ID NO: 266 or 267, where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the CDR-H2 of the antibody comprises a proline (P) at position 3, a tryptophan (W) at position 4, and a serine (S) or asparagine (N) at position 5.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the CDR-H2 of the antibody comprises a proline (P) at position 3, a tryptophan (W) at position 4, and a serine (S) or asparagine (N) at position 5.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, where the heavy chain variable domain comprising SEQ ID NO: 234 and the light chain variable domain comprising SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the CDR-H2 of the antibody comprises a proline (P) at position 3, a tryptophan (W) at position 4, and a serine (S) or asparagine (N) at position 5.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, where the heavy chain variable domain comprising SEQ ID NO: 234 and the light chain variable domain comprising SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).
a CDR-L3 amino acid sequence comprising SEQ ID NO: 195 or having 1, 2, 3, 4 or 5 amino acid alterations relative to SEQ ID NO:195 (3B2g2m1).

In an embodiment, the antibody-based molecule that binds to human muscle-specific tyrosine-protein kinase (MuSK) comprises wild-type human IgG constant Fc region, a heavy chain variable domain and a light chain variable domain,

where the wild-type human IgG constant Fc region comprising at least 80% sequence identity to SEQ ID NO: 266 or 267, wherein a), wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A, and
where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises wild-type human IgG constant Fc region, a heavy chain variable domain and a light chain variable domain,

where the wild-type human IgG constant Fc region comprising SEQ ID NO: 266 or 267, wherein L234A and/or L235A substitution(s) is(are) numbered according the EU numbering system introduced into said Fc region, and
where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, wherein L234A and L235A substitutions numbered according the EU numbering system are introduced into said Fc region, and

where the heavy chain variable domain comprising SEQ ID NO: 234 and the light chain variable domain comprising SEQ ID NO: 235, and
where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).
a CDR-L3 amino acid sequence comprising SEQ ID NO: 195 or having 1, 2, 3, 4 or 5 amino acid alterations relative to SEQ ID NO:195 (3B2g2m1).

In an embodiment, the anti-MuSK antibody or antigen binding fragment thereof, comprises:
a) A full length heavy chain comprising an amino acid sequence that is at least 80% identical or similar to SEQ ID NO: 268 and
b) A full length light chain comprising an amino acid sequence that is at least 80% identical or similar to SEQ ID NO: 269, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the anti-MuSK antibody or antigen binding fragment thereof, comprises:
a) A full length heavy chain comprising SEQ ID NO: 268 and
b) A full length light chain comprising SEQ ID NO: 269, and
c) Wherein the full length heavy chain comprises L234A and L235A mutations numbered according the EU numbering system.

In an embodiment, the anti-MuSK antibody or antigen binding fragment thereof, comprises:
a) A full length heavy chain comprising an amino acid sequence that is at least 80% identical or similar to SEQ ID NO: 270 and
b) A full length light chain comprising an amino acid sequence that is at least 80% identical or similar to SEQ ID NO: 271, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the anti-MuSK antibody or antigen binding fragment thereof, comprises:
a) A full length heavy chain comprising SEQ ID NO: 270 and
b) A full length light chain comprising SEQ ID NO: 271, and
c) Wherein the full length heavy chain comprises L234A and L235A mutations numbered according the EU numbering system.

### Polynucleotides

Another aspect of the present invention is directed to isolated polynucleotides encoding the MuSK antibody-based molecules described herein. In one embodiment, the polynucleotide encoding the MuSK antibody of the present invention comprises a nucleotide sequence encoding any one, any two, any three, any four, any five, or any six of the CDRs described supra, including the heavy chain CDRs of SEQ ID NOs: 1-48, 135-140, 147-158, 240-251 and the light chain CDRs of SEQ ID NOs: 49-96, 141-146, and 159-195.

Accordingly, the invention provides a polynucleotide for use in the treatment of a neuromuscular disease in a human subject, which polynucleotide comprises a nucleotide sequence encoding the anti-MuSK antibody or antigen binding fragment or VH, VL or CDRs domain thereof.

In an embodiment, the polynucleotide comprises a nucleotide sequence encoding a VH domain, where the VH domain comprises (i) a heavy chain variable domain comprising the CDR-H1 of SEQ ID NO: 1, the CDR-H2 of SEQ ID NO: 17, and the CDR-H3 of SEQ ID NO: 33; (ii) a heavy chain variable domain comprising the CDR-H1 of SEQ ID NO: 2, the CDR-H2 of SEQ ID NO: 18, and the CDR-H3 of SEQ ID NO: 34; (iii) a heavy chain variable domain comprising the CDR-H1 of SEQ ID NO: 3, the CDR-H2 of SEQ ID NO: 19, and the CDR-H3 of SEQ ID NO: 35; (iv) a heavy chain variable domain comprising the CDR-H1 of SEQ ID NO: 4, the CDR-H2 of SEQ ID NO: 20, and the CDR-H3 of SEQ ID NO: 36; (v) a heavy chain variable domain comprising the CDR-H1 of SEQ ID NO: 5, the CDR-H2 of SEQ ID NO: 21, and the CDR-H3 of SEQ ID NO: 37; (vi) a heavy chain variable domain comprising the CDR-H1 of SEQ ID NO: 6, the CDR-H2 of SEQ ID NO: 22, and the CDR-H3 of SEQ ID NO: 38; (vii) a heavy chain variable domain comprising the CDR-H1 of SEQ ID NO: 7, the CDR-H2 of SEQ ID NO: 23, and the CDR-H3 of SEQ ID NO: 39; (viii) a heavy chain variable domain comprising the CDR-H1 of SEQ ID NO: 8, the CDR-H2 of SEQ ID NO: 24, and the CDR-H3 of SEQ ID NO: 40; (ix) a heavy chain variable domain comprising the CDR-H1 of SEQ ID NO: 9, the CDR-H2 of SEQ ID NO: 25, and the CDR-H3 of SEQ ID NO: 41; (x) a heavy chain variable domain comprising the CDR-H1 of SEQ ID NO: 10, the CDR-H2 of SEQ ID NO: 26, and the CDR-H3 of SEQ ID NO: 42; (xi) a heavy chain variable domain comprising the CDR-H1 of SEQ ID NO: 11, the CDR-H2 of SEQ ID NO: 27, and the CDR-H3 of SEQ ID NO: 43; (xii) a heavy chain variable domain comprising the CDR-H1 of SEQ ID NO: 12, the CDR-H2 of SEQ ID NO: 28, and the CDR-H3 of SEQ ID NO: 44; (xiii) a heavy chain variable domain comprising the CDR-H1 of SEQ ID NO: 13, the CDR-H2 of SEQ ID NO: 29, and the CDR-H3 of SEQ ID NO: 45; (xiv) a heavy chain variable domain comprising the CDR-H1 of SEQ ID NO: 14, the CDR-H2 of SEQ ID NO: 30, and the CDR-H3 of SEQ ID NO: 46; (xv) a heavy chain variable domain comprising the CDR-H1 of SEQ ID NO: 15, the CDR-H2 of SEQ ID NO: 31, and the CDR-H3 of SEQ ID NO: 47; (xvi) a heavy chain variable domain comprising the CDR-H1 of SEQ ID NO: 16, the CDR-H2 of SEQ ID NO: 32, and the CDR-H3 of SEQ ID NO: 48; (xvii) a heavy chain variable domain comprising the CDR-H1 of SEQ ID NO: 135, the CDR-H2 of SEQ ID NO: 137, and the CDR-H3 of SEQ ID NO: 139; and (xviii) a heavy chain variable domain comprising the CDR-H1 of SEQ ID NO: 136, the CDR-H2 of SEQ ID NO: 138, and the CDR-H3 of SEQ ID NO: 140.

In some embodiments, the polynucleotide comprises a nucleotide sequence encoding a VH domain, where the VH domain comprises (ii.a) a heavy chain variable domain comprising the CDR-H1 of SEQ ID NO: 2, the CDR-H2 of SEQ ID NO: 18, and the CDR-H3 of SEQ ID NO: 240 (X2m1); (ii.b) a heavy chain variable domain comprising the CDR-H1 of SEQ ID NO: 2, the CDR-H2 of SEQ ID NO: 18, and the CDR-H3 of SEQ ID NO: 241 (X2m2); (ii.c) a heavy chain variable domain comprising the CDR-H1 of SEQ ID NO: 2, the CDR-H2 of SEQ ID NO: 18, and the CDR-H3 of SEQ ID NO: 242 (X2m3); (ii.d) a heavy chain variable domain comprising the CDR-H1 of SEQ ID NO: 2, the CDR-H2 of SEQ ID NO: 18, and the CDR-H3 of SEQ ID NO: 243 (X2m4); (ii.e) a heavy chain variable domain comprising the CDR-H1 of SEQ ID NO: 2, the CDR-H2 of SEQ ID NO: 18, and the CDR-H3 of SEQ ID NO: 244 (X2m5); (ii.f) a heavy chain variable domain comprising the CDR-H1 of SEQ ID NO: 2, the CDR-H2 of SEQ ID NO: 18, and the CDR-H3 of SEQ ID NO: 245 (X2m6); (ii.g) a heavy chain variable domain comprising the CDR-H1 of SEQ ID NO: 2, the CDR-H2 of SEQ ID NO: 18, and the CDR-H3 of SEQ ID NO: 246 (X2m7); (ii.h) a heavy chain variable domain comprising the CDR-H1 of SEQ ID NO: 2, the CDR-H2 of SEQ ID NO: 18, and the CDR-H3 of SEQ ID NO: 247 (X2m8).

In some embodiments, the polynucleotide comprises a nucleotide sequence encoding a VH domain, where the VH domain comprises (xvii.a) a heavy chain variable domain comprising the CDR-H1 of SEQ ID NO: 135, the CDR-H2 of SEQ ID NO: 137, and the CDR-H3 of SEQ ID NO: 248 (X17m1); (xvii.b) a heavy chain variable domain comprising the CDR-H1 of SEQ ID NO: 135, the CDR-H2 of SEQ ID NO: 137, and the CDR-H3 of SEQ ID NO: 249(X17m2); (xvii.c) a heavy chain variable domain comprising the CDR-H1 of SEQ ID NO: 135, the CDR-H2 of SEQ ID NO: 137, and the CDR-H3 of SEQ ID NO: 250 (X17m3); (xvii.d) a heavy chain variable domain comprising the CDR-H1 of SEQ ID NO: 135, the CDR-H2 of SEQ ID NO: 137, and the CDR-H3 of SEQ ID NO: 251 (X17m6).

In an embodiment, the polynucleotide comprises a nucleotide sequence encoding a VH domain, where the VH domain comprises: (xix) a heavy chain variable domain comprising the CDR-H1 of SEQ ID NO: 147, the CDR-H2 of SEQ ID NO: 150, and the CDR-H3 of SEQ ID NO: 156; (xx) a heavy chain variable domain comprising the CDR-H1 of SEQ ID NO: 148, the CDR-H2 of SEQ ID NO: 151, and the CDR-H3 of SEQ ID NO: 157; (xxi) a heavy chain variable domain comprising the CDR-H1 of SEQ ID NO: 149, the CDR-H2 of SEQ ID NO: 152, and the CDR-H3 of SEQ ID NO: 158.

In an embodiment, the polynucleotide comprises a nucleotide sequence encoding a VH domain, where the VH domain comprises: (xxii) a heavy chain variable domain comprising the CDR-H1 of SEQ ID NO: 147, the CDR-H2 of SEQ ID NO: 153, and the CDR-H3 of SEQ ID NO:156; (xxiii) a heavy chain variable domain comprising the CDR-H1 of SEQ ID NO: 147, the CDR-H2 of SEQ ID NO: 154, and the CDR-H3 of SEQ ID NO: 156; (xxiv) a heavy chain variable domain comprising the CDR-H1 of SEQ ID NO: 147, the CDR-H2 of SEQ ID NO: 155, and the CDR-H3 of SEQ ID NO: 156.

In an embodiment, the polynucleotide comprises a nucleotide sequence encoding a VL domain, where the VL domain comprises (i) a light chain variable domain comprising the CDR-L1 of SEQ ID NO: 49, the CDR-L2 of SEQ ID NO: 65, and the CDR-L3 of SEQ ID NO: 81; (ii) a light chain variable domain comprising the CDR-L1 of SEQ ID NO: 50, the CDR-L2 of SEQ ID NO: 66, and the CDR-L3 of SEQ ID NO: 82; (iii) a light chain variable domain comprising the CDR-L1 of SEQ ID NO: 51, the CDR-L2 of SEQ ID NO: 67, and the CDR-L3 of SEQ ID NO: 83; (iv) a light chain variable domain comprising the CDR-L1 of SEQ ID NO: 52, the CDR-L2 of SEQ ID NO: 68, and the CDR-L3 of SEQ ID NO: 84; (v) a light chain variable domain comprising the CDR-L1 of SEQ ID NO: 53, the CDR-L2 of SEQ ID NO: 69, and the CDR-L3 of SEQ ID NO: 85; (vi) a light chain variable domain comprising the CDR-L1 of SEQ ID NO: 54, the CDR-L2 of SEQ ID NO: 70, and the CDR-L3 of SEQ ID NO: 86; (vii) a light chain variable domain comprising the CDR-L1 of SEQ ID NO: 55, the CDR-L2 of SEQ ID NO:71, and the CDR-L3 of SEQ ID NO: 87; (viii) a light chain variable domain comprising the CDR-L1 of SEQ ID NO: 56, the CDR-L2 of SEQ ID NO: 72, and the CDR-L3 of SEQ ID NO: 88; (ix) a light chain variable domain comprising the CDR-L1 of SEQ ID NO: 57, the CDR-L2 of SEQ ID NO: 73, and the CDR-L3 of SEQ ID NO: 89; (x) a light chain variable domain comprising the CDR-L1 of SEQ ID NO: 58, the CDR-L2 of SEQ ID NO: 74, and the CDR-L3 of SEQ ID NO: 90; (xi) a light chain variable domain comprising the CDR-L1 of SEQ ID NO: 59, the CDR-L2 of SEQ ID NO: 75, and the CDR-L3 of SEQ ID NO: 91; (xii) a light chain variable domain comprising the CDR-L1 of SEQ ID NO: 60, the CDR-L2 of SEQ ID NO: 76, and the CDR-L3 of SEQ ID NO: 92; (xiii) a light chain variable domain comprising the CDR-L1 of SEQ ID NO: 61, the CDR-L2 of SEQ ID NO: 77, and the CDR-L3 of SEQ ID NO: 93; (xiv) a light chain variable domain comprising the CDR-L1 of SEQ ID NO: 62, the CDR-L2 of SEQ ID NO: 78, and the CDR-L3 of SEQ ID NO: 94; (xv) a light chain variable domain comprising the CDR-L1 of SEQ ID NO: 63, the CDR-L2 of SEQ ID NO: 79, and the CDR-L3 of SEQ ID NO: 95; (xvi) a light chain variable domain comprising the CDR-L1 of SEQ ID NO: 64, the CDR-L2 of SEQ ID NO: 80, and the CDR-L3 of SEQ ID NO: 96; (xvii) a light chain variable domain comprising the CDR-L1 of SEQ ID NO: 141, the CDR-L2 of SEQ ID NO: 143, and the CDR-L3 of SEQ ID NO: 145; and (xviii) a light chain variable domain comprising the CDR-L1 of SEQ ID NO: 142, the CDR-L2 of SEQ ID NO: 144, and the CDR-L3 of SEQ ID NO: 146.

In an embodiment, the polynucleotide comprises a nucleotide sequence encoding a VL domain, where the VL domain comprises (xix) a light chain variable domain comprising the CDR-L1 of SEQ ID NO: 159, the CDR-L2 of SEQ ID NO: 170, and the CDR-L3 of SEQ ID NO: 180; (xx) a light chain variable domain comprising the CDR-L1 of SEQ ID NO: 159, the CDR-L2 of SEQ ID NO: 171, and the CDR-L3 of SEQ ID NO: 181; (xxi) a light chain variable domain comprising the CDR-L1 of SEQ ID NO: 160, the CDR-L2 of SEQ ID NO: 172, and the CDR-L3 of SEQ ID NO: 182; (xxii) a light chain variable domain comprising the CDR-L1 of SEQ ID NO: 159, the CDR-L2 of SEQ ID NO: 172, and the CDR-L3 of SEQ ID NO: 183; (xxiii) a light chain variable domain comprising the CDR-L1 of SEQ ID NO: 159, the CDR-L2 of SEQ ID NO: 171, and the CDR-L3 of SEQ ID NO: 184; (xxiv) a light chain variable domain comprising the CDR-L1 of SEQ ID NO: 159, the CDR-L2 of SEQ ID NO: 173, and the CDR-L3 of SEQ ID NO: 185; (xxv) a light chain variable domain comprising the CDR-L1 of SEQ ID NO: 159, the CDR-L2 of SEQ ID NO: 173, and the CDR-L3 of SEQ ID NO: 186; (xxvi) a light chain variable domain comprising the CDR-L1 of SEQ ID NO: 161, the CDR-L2 of SEQ ID NO: 174, and the CDR-L3 of SEQ ID NO: 187; (xxvii) a light chain variable domain comprising the CDR-L1 of SEQ ID NO: 162, the CDR-L2 of SEQ ID NO: 174, and the CDR-L3 of SEQ ID NO: 188; (xxviii) a light chain variable domain comprising the CDR-L1 of SEQ ID NO: 163, the CDR-L2 of SEQ ID NO: 174, and the CDR-L3 of SEQ ID NO: 188; (xxix) a light chain variable domain comprising the CDR-L1 of SEQ ID NO: 164, the CDR-L2 of SEQ ID NO: 174, and the CDR-L3 of SEQ ID NO: 189; (xxx) a light chain variable domain comprising the CDR-L1 of SEQ ID NO: 165, the CDR-L2 of SEQ ID NO: 175, and the CDR-L3 of SEQ ID NO: 190; (xxxi) a light chain variable domain comprising the CDR-L1 of SEQ ID NO: 166, the CDR-L2 of SEQ ID NO: 176, and the CDR-L3 of SEQ ID NO: 191; (xxxi) a light chain variable domain comprising the CDR-L1 of SEQ ID NO: 167, the CDR-L2 of SEQ ID NO: 177, and the CDR-L3 of SEQ ID NO: 192; (xxxii) a light chain variable domain comprising the CDR-L1 of SEQ ID NO: 168, the CDR-L2 of SEQ ID NO: 178, and the CDR-L3 of SEQ ID NO: 193; (xxxiii) a light chain variable domain comprising the CDR-L1 of SEQ ID NO: 169, the CDR-L2 of SEQ ID NO: 179, and the CDR-L3 of SEQ ID NO: 194.

In an embodiment, the polynucleotide comprises a nucleotide sequence encoding a VL domain, where the VL domain comprises (xxxiv) a light chain variable domain comprising the CDR-L1 of SEQ ID NO: 159, the CDR-L2 of SEQ ID NO: 172, and the CDR-L3 of SEQ ID NO: 183; (xxxv) a light chain variable domain comprising the CDR-L1 of SEQ ID NO: 159, the CDR-L2 of SEQ ID NO: 172, and the CDR-L3 of SEQ ID NO: 195.

In an embodiment, the isolated polynucleotide encoding the MuSK antibody based molecule encodes any one of the VH and/or VL domain sequences as provided in Table 3 infra. The nucleic acid molecules described herein include isolated polynucleotides, portions of expression vectors or portions of linear DNA sequences, including linear DNA sequences used for in vitro transcription/translation, and vectors compatible with prokaryotic, eukaryotic or filamentous phage expression, secretion, and/or display of the antibodies or binding fragments thereof described herein.

In a preferred embodiment, the polynucleotide comprises a nucleotide sequence encoding a VH that comprises an amino acid sequence that is at least 80% identical or similar to SEQ ID NO: 234. In another preferred embodiment, the polynucleotide comprises a nucleotide sequence encoding a VL that comprises an amino acid sequence that is at least 80% identical or similar to SEQ ID NO: 235

In a preferred embodiment, the polynucleotide comprises a nucleotide sequence encoding an antibody-based molecule that binds to human muscle-specific tyrosine-protein kinase (MuSK), said molecule comprising a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the polynucleotide comprises a nucleotide sequence encoding an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the polynucleotide comprises or consists of a nucleotide sequence that is at least 80% 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 264. In an even more preferred embodiment, the polynucleotide comprises or consists of SEQ ID NO:264.

In another more preferred embodiment, the polynucleotide comprises or consists of a nucleotide sequence that is at least 80% 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 265. In an even more preferred embodiment, the polynucleotide comprises or consists of SEQ ID NO:265.

In a more preferred embodiment, the polynucleotide comprises a nucleotide sequence encoding an antibody-based molecule that binds to human muscle-specific tyrosine-protein kinase (MuSK), that comprises a heavy chain variable domain and a light chain variable domain, wherein the heavy chain variable domain is encoded by a nucleotide sequence that is at least 80% identical to SEQ ID NO: 264 and the light chain variable domain is encoded by a nucleotide sequence that is at least 80% identical to SEQ ID NO: 265. In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:276 encoding the full length heavy chain, and
b) a nucleotide sequence that is at least 80% identical to SEQ ID NO:278 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:277 encoding the heavy chain variable domain, and
b) a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:276 encoding the full length heavy chain, wherein said nucleotide sequence comprising a nucleotide sequence that is at least 80% identical to SEQ ID NO: 277 encoding the heavy chain variable domain, and
b) a nucleotide sequence that is at least 80% identical to SEQ ID NO:278 encoding the full length light chain, wherein said nucleotide sequence comprising a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence SEQ ID NO:276 and
b) a nucleotide sequence SEQ ID NO:278.

The polynucleotides of the invention may be produced by chemical synthesis such as solid phase polynucleotide synthesis on an automated polynucleotide synthesizer and assembled into complete single or double stranded molecules. Alternatively, the polynucleotides may be produced by other techniques such a PCR followed by routine cloning. Techniques for producing or obtaining polynucleotides of a given sequence are well known in the art.

The polynucleotides may comprise at least one non-coding sequence, such as a promoter or enhancer sequence, intron, polyadenylation signal, a cis sequence facilitating RepA binding, and the like. The polynucleotide sequences may also comprise additional sequences encoding for example a linker sequence, a marker or a tag sequence, such as a histidine tag or an HA tag to facilitate purification or detection of the protein, a signal sequence, a fusion protein partner such as RepA, Fc portion, or bacteriophage coat protein such as pIX or pIll.

### Vector

In another aspect, there is provided a vector (preferably an expression vector) for use in the treatment of a neuromuscular disorder in a human subject comprising the polynucleotide encoding the MuSK antibody-based molecule (or Anti-MuSK antibody or antigen binding fragment thereof) as described herein.

Such vectors include, without limitation, plasmid vectors, viral vectors, including without limitation, vaccina vector, lentiviral vector, adenoviral vector, adeno-associated viral vector, vectors for baculovirus expression, transposon based vectors or any other vector suitable for introduction of the polynucleotides described herein into a given organism or genetic background by any means to facilitate expression of the encoded antibody polypeptide. In one embodiment, the polynucleotide encoding the heavy chain variable domain, alone or together with the polynucleotide encoding the light chain variable domain as described herein, are combined with sequences of a promoter, a translation initiation segment (e.g., a ribosomal binding sequence and start codon), a 3′ untranslated region, polyadenylation signal, a termination codon, and transcription termination to form one or more expression vector constructs.

In one embodiment, the vector is an adenoviral-associated viral (AAV) vector. A number of therapeutic AAV vectors suitable for delivery of the polynucleotides encoding antibodies described herein to the central nervous system are known in the art. See e.g., Deverman et al., “Gene Therapy for Neurological Disorders: Progress and Prospects,” Nature Rev. 17:641-659 (2018), which in hereby incorporated by reference in its entirety. Suitable AAV vectors include serotypes AAV1, AAV2, AAV3, AAV4, AAV5, AAV6, AAV7, AAV8, AAV9, AAV10, or AAV11 in their native form or engineered for enhanced tropism. AAV vectors known to have tropism for the CNS that are particularly suited for therapeutic expression of the MuSK antibodies described herein include, AAV1, AAV2, AAV4, AAV5, AAV8 and AAV9 in their native form or engineered for enhanced tropism. In one embodiment, the AAV vector is an AAV2 vector. In another embodiment, the AAV vector is an AAV5 vector (Vitale et al., “Anti-tau Conformational scFv MC1 Antibody Efficiently Reduces Pathological Tau Species in Adult JNPL3 Mice,” Acta Neuropathol. Commun. 6:82 (2018), which is hereby incorporate by reference in its entirety). In another embodiment, the AAV vector is an AAV9 vector (Haiyan et al., “Targeting Root Cause by Systemic scAAV9-hlDS Gene Delivery: Functional Correction and Reversal of Severe MPSII in Mice,” Mol. Ther. Methods Clin. Dev. 10:327-340 (2018), which is hereby incorporated by reference in its entirety). In another embodiment, the AAV vector is an AAVrh10 vector (Liu et al., “Vectored Intracerebral Immunizations with the Anti-Tau Monoclonal Antibody PHF1 Markedly Reduces Tau Pathology in Mutant Transgenic Mice,” J. Neurosci. 36(49): 12425-35 (2016), which is hereby incorporated by reference in its entirety).

In another embodiment the AAV vector is a hybrid vector comprising the genome of one serotype, e.g., AAV2, and the capsid protein of another serotype, e.g., AAV1 or AAV3-9 to control tropism. See e.g., Broekman et al., “Adeno-associated Virus Vectors Serotyped with AAV8 Capsid are More Efficient than AAV-1 or −2 Serotypes for Widespread Gene Delivery to the Neonatal Mouse Brain,” Neuroscience 138:501-510 (2006), which is hereby incorporated by reference in its entirety. In one embodiment, the AAV vector is an AAV2/8 hybrid vector (Ising et al., “AAV-mediated Expression of Anti-Tau ScFv Decreases Tau Accumulation in a Mouse Model of Tauopathy,” J. Exp. Med. 214(5):1227 (2017), which is hereby incorporated by reference in its entirety). In another embodiment the AAV vector is an AAV2/9 hybrid vector (Simon et al., “A Rapid Gene Delivery-Based Mouse Model for Early-Stage Alzheimer Disease-Type Tauopathy,” J. Neuropath. Exp. Neurol. 72(11): 1062-71 (2013), which is hereby incorporated by reference in its entirety).

In another embodiment, the AAV vector is one that has been engineered or selected for its enhanced CNS transduction after intraparenchymal administration, e.g., AAV-DJ (Grimm et al., J. Viol. 82:5887-5911 (2008), which is hereby incorporated by reference in its entirety); increased transduction of neural stem and progenitor cells, e.g., SCH9 and AAV4.18 (Murlidharan et al., J. Virol. 89: 3976-3987 (2015) and Ojala et al., Mol. Ther. 26:304-319 (2018), which are hereby incorporated by reference in their entirety); enhanced retrograde transduction, e.g., rAAV2-retro (Muller et al., Nat. Biotechnol. 21:1040-1046 (2003), which is hereby incorporated by reference in its entirety); selective transduction into brain endothelial cells, e.g., AAV-BRI (Korbelin et al., EMBO Mol. Med. 8: 609-625 (2016), which is hereby incorporated by reference in its entirety); or enhanced transduction of the adult CNS after IV administration, e.g., AAV-PHP.B and AAVPHP.eB (Deverman et al., Nat. Biotechnol. 34: 204-209 (2016) and Chan et al., Nat. Neurosci. 20: 1172-1179 (2017), which are hereby incorporated by reference in their entirety.

In accordance with this embodiment, the expression vector construct encoding the MuSK antibody-based molecule includes the polynucleotide encoding the heavy chain polypeptide, a functional fragment thereof, a variant thereof, or combinations thereof. The expression construct can alternatively include a nucleic acid sequence encoding the light chain polypeptide, a functional fragment thereof, a variant thereof, or combinations thereof. In an embodiment, the expression vector construct includes a nucleic acid sequence encoding the heavy chain polypeptide, a functional fragment thereof, or a variant thereof, and the light chain polypeptide, a functional fragment thereof, or a variant thereof.

In an embodiment, the expression construct further comprises a promoter sequence suitable for driving expression of the MuSK antibody-based molecule. Suitable promoter sequences include, without limitation, the elongation factor 1-alpha promoter (EF1a) promoter, a phosphoglycerate kinase-1 promoter (PGK) promoter, a cytomegalovirus immediate early gene promoter (CMV), a chimeric liver-specific promoter (LSP), a cytomegalovirus enhancer/chicken beta-actin promoter (CAG), a tetracycline responsive promoter (TRE), a transthyretin promoter (TTR), a simian virus 40 promoter (SV40) and a CK6 promoter. Other promoters suitable for driving gene expression in mammalian cells that are known in the art are also suitable for incorporation into the expression constructs disclosed herein.

In an embodiment, the expression construct (or expression vector) further encodes a linker sequence. The linker sequence can encode an amino acid sequence that spatially separates and/or links the one or more components of the expression construct (heavy chain and light chain components of the encoded antibody).

In a preferred embodiment, the expression vector comprises a polynucleotide that encodes an antibody-based molecule that binds to human muscle-specific tyrosine-protein kinase (MuSK) and that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the expression vector comprises a nucleotide encoding an antibody-based molecule that binds to human muscle-specific tyrosine-protein kinase (MuSK) and comprising a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:276 encoding the full length heavy chain, and
b) a nucleotide sequence that is at least 80% identical to SEQ ID NO:278 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:277 encoding the heavy chain variable domain, and
b) a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
c) a nucleotide sequence that is at least 80% identical to SEQ ID NO:276 encoding the full length heavy chain, wherein said nucleotide sequence comprising a nucleotide sequence that is at least 80% identical to SEQ ID NO: 277 encoding the heavy chain variable domain, and
d) a nucleotide sequence that is at least 80% identical to SEQ ID NO:278 encoding the full length light chain, wherein said nucleotide sequence comprising a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence SEQ ID NO:276 and
b) a nucleotide sequence SEQ ID NO:278.

### Host Cell

Another aspect of the present invention is a host cell or cell-free expression system for use in the treatment of a neuromuscular disease in a human subject, wherein the cell contains the expression vector encoding the MuSK antibodies (or antigen binding fragment thereof) and optionally producing said MuSK antibodies as described herein.

The MuSK antibody-based molecules described herein can optionally be produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety).

In some embodiments, the host cell chosen for expression may be of mammalian origin. Suitable mammalian host cells include, without limitation, COS-1 cells, COS-7 cells, HEK293 cells, BHK21 cells, CHO cells, BSC-1 cells, HeG2 cells, SP2/0 cells, HeLa cells, mammalian myeloma cells, mammalian lymphoma cells, or any derivative, immortalized or transformed cell thereof. Other suitable host cells include, without limitation, yeast cells, insect cells, and plant cells. Alternatively, the host cell may be selected from a species or organism incapable of glycosylating polypeptides, e.g., a prokaryotic cell or organism, such as BL21, BL21(DE3), BL21-GOLD(DE3), XL1-Blue, JM109, HMS174, HMS174(DE3), and any of the natural or engineered E. coli spp, Klebsiella spp., or Pseudomonas spp strains.

The MuSK antibody-based molecules described herein can be prepared by any of a variety of techniques using the isolated polynucleotides, vectors, and host cells described supra. In general, antibodies can be produced by cell culture techniques, including the generation of monoclonal antibodies via conventional techniques, or via transfection of antibody genes, heavy chains and/or light chains into suitable bacterial or mammalian cell hosts, in order to allow for the production of antibodies, wherein the antibodies may be recombinant. In an embodiment, the MuSK antibody-based molecule described herein is a monoclonal antibody or functional binding fragment thereof. Standard molecular biology techniques are used to prepare the recombinant expression vector, transfect the host cells, select for transformants, culture the host cells and recover the antibody from the culture medium. Transfecting the host cell can be carried out using a variety of techniques commonly used for the introduction of exogenous DNA into a prokaryotic or eukaryotic host cell, e.g., by electroporation, calcium-phosphate precipitation, DEAE-dextran transfection and the like. Although it is possible to express the antibodies described herein in either prokaryotic or eukaryotic host cells, expression of antibodies in eukaryotic cells, in particular mammalian cells is sometimes preferable, because such eukaryotic cells (and in particular mammalian cells) are more likely than prokaryotic cells to assemble and secrete a properly folded and immunologically active antibody.

As noted above, exemplary mammalian host cells for expressing the recombinant antibodies of the invention include Chinese Hamster Ovary (CHO cells) (including dhfr-CHO cells, described in Urlaub and Chasin, Proc. Natl. Acad. Sci. USA, 77: 4216-4220 (1980), which is hereby incorporated by reference in its entirety). Other suitable mammalian host cells include, without limitation, NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown.

Host cells can also be used to produce functional antibody fragments, such as Fab fragments or scFv molecules. It is understood that variations on the above procedure are within the scope of the present invention. For example, it may be desirable to transfect a host cell with DNA encoding functional fragments of either the light chain and/or the heavy chain of an antibody described herein. Recombinant DNA technology may also be used to remove some or all of the DNA encoding either or both of the light and heavy chains that is not necessary for binding to the antigens of interest. The molecules expressed from such truncated DNA molecules are also encompassed by the antibodies described herein.

The antibodies and antibody binding fragments are recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

In a preferred embodiment, the host cell expresses an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the host cell expresses an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:276 encoding the full length heavy chain, and
b) a nucleotide sequence that is at least 80% identical to SEQ ID NO:278 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:277 encoding the heavy chain variable domain, and
b) a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:276 encoding the full length heavy chain, wherein said nucleotide sequence comprising a nucleotide sequence that is at least 80% identical to SEQ ID NO: 277 encoding the heavy chain variable domain, and
b) a nucleotide sequence that is at least 80% identical to SEQ ID NO:278 encoding the full length light chain, wherein said nucleotide sequence comprising a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence SEQ ID NO:276 and
b) a nucleotide sequence SEQ ID NO:278.

### Compositions Comprising MuSK Antibody-Based Molecules

The MuSK antibody-based molecules or polynucleotide encoding the MuSK antibody-based molecules of the present invention may be advantageously administered as compositions.

Therefore in a further aspect, there is provided a composition for use in the treatment of a neuromuscular disorder in a human subject, said composition comprising an antibody or antigen binding fragment, a polynucleotide, an expression vector or a host cell or cell free expression system as defined herein.

In an embodiment, said composition is a pharmaceutical composition. In an embodiment, said pharmaceutical composition comprising at least one pharmaceutically acceptable carrier or excipients.

In an embodiment, such compositions are pharmaceutical compositions comprising an active therapeutic agent (i.e., the MuSK antibody) and one or more of a variety of other pharmaceutically acceptable components. See REMINGTON: THE SCIENCE AND PRACTICE OF PHARMACY (21st Edition) (2005) (Troy, D. B. et al. (Eds.) Lippincott Williams & Wilkins (Publs.), Baltimore MD), which is hereby incorporated by reference in its entirety. The preferred form depends on the intended mode of administration and therapeutic application. The compositions can also include, depending on the formulation desired, pharmaceutically acceptable, non-toxic carriers, excipients, diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition, and which are vehicles commonly used to formulate pharmaceutical compositions for animal or human administration. The diluent is selected to not affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, or non-toxic, nontherapeutic, non-immunogenic stabilizers and the like. Examples of suitable aqueous and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well-known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

The compositions may also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Suitability for carriers and other components of pharmaceutical compositions is determined based on the lack of significant negative impact on the desired biological properties of the active antibody-based molecule of the present invention (e.g., less than a substantial impact (e.g., 10% or less relative inhibition, 5% or less relative inhibition, etc.) on antigen binding).

The pharmaceutical compositions of the present invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. The pharmaceutical compositions of the present invention may also comprise isotonicity agents, such as sugars, polyalcohols, such as mannitol, sorbitol, glycerol or sodium chloride in the compositions. The pharmaceutical compositions of the present invention may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The antibodies of the present invention may be prepared with carriers that will protect the antibodies against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well-known in the art. Methods for the preparation of such formulations are generally known to those skilled in the art. See, e.g., SUSTAINED AND CONTROLLED RELEASE DRUG DELIVERY SYSTEMS, J. R. Robinson, ed., Marcel Dekker, Inc., New York, 1978. In one embodiment, the antibodies of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art.

Pharmaceutical compositions for injection must typically be sterile and stable under the conditions of manufacture and storage. The composition may be formulated as a solution, microemulsion, liposome, or other ordered structure suitable to achieve high drug concentration. The carrier may be an aqueous or non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

For parenteral administration, agents of the present invention are typically formulated as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the invention can be administered in the form of a depot injection or implant preparation which can be formulated in such a manner as to permit a sustained release of the active ingredient.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles, such as polylactide, polyglycolide, or copolymer, for enhanced adjuvant effect (Langer, et al., Science 249:1527 (1990); Hanes, et al., Advanced Drug Delivery Reviews 28:97-119 (1997), which are hereby incorporated by reference in their entirety). Additional formulations suitable for other modes of administration include oral, intranasal, and pulmonary formulations, suppositories, and transdermal applications.

In an embodiment, the composition comprises the anti-MuSK antibody (or antigen-binding fragment) which comprises a heavy chain variable domain comprising the CDR-H1 of SEQ ID NO: 147, the CDR-H2 of SEQ ID NO: 153, and the CDR-H3 of SEQ ID NO: 156, and a light chain variable domain comprising the CDR-L1 of SEQ ID NO: 159, the CDR-L2 of SEQ ID NO: 172, and the CDR-L3 of SEQ ID NO: 195 (3B2g2m1).

In an embodiment, the composition comprises the anti-MuSK antibody (or antigen-binding fragment) which comprises a heavy chain variable domain comprising the CDR-H1 of SEQ ID NO: 147, the CDR-H2 of SEQ ID NO: 153, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m1).

In an embodiment, the anti-MuSK antibody (or antigen-binding fragment) which comprises a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m2).

In an embodiment, the anti-MuSK antibody (or antigen-binding fragment) which comprises a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m2).

In an embodiment, the anti-MuSK antibody (or antigen-binding fragment) which comprises a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2).

In a preferred embodiment, the composition comprises an antibody or antigen-binding fragment that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO:156 or having 1, 2, 3, 4 or 5 amino acid alterations relative to SEQ ID NO:156 (3B2g2m1) and
   where the light chain variable domain comprises:
a CDR-L1 amino acid sequence comprises SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, composition comprises an antibody or antigen-binding fragment that binds to human muscle-specific tyrosine-protein kinase (MuSK) that comprises wild-type human IgG constant Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises
an amino acid sequence that is at least 80% identical or similar to SEQ ID NO: 235, and
   where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK) that comprises wild-type human IgG constant Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the composition comprises an antibody or antigen-binding fragment that binds to human muscle-specific tyrosine-protein kinase (MuSK) that comprises wild-type human IgG constant Fc region wherein L234A and/or L235A substitution(s) numbered according the EU numbering system is(are) introduced into said Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK) that comprises wild-type human IgG constant Fc region wherein L234A and/or L235A substitution(s) numbered according the EU numbering system is(are) introduced into said Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the composition comprises an antibody or antigen-binding fragment that binds to human muscle-specific tyrosine-protein kinase (MuSK) that comprises:
a) A full length heavy chain comprising an amino acid sequence that is at least 80% identical or similar to SEQ ID NO: 268 and
b) A full length light chain comprising an amino acid sequence that is at least 80% identical or similar to SEQ ID NO: 269, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the composition comprises an antibody or antigen-binding fragment that binds to human muscle-specific tyrosine-protein kinase (MuSK) that comprises:
a) A full length heavy chain comprising SEQ ID NO: 268 and
b) A full length light chain comprising SEQ ID NO: 269, and
c) Wherein the full length heavy chain comprises L234A and L235A mutations numbered according the EU numbering system.

In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

In an embodiment, the composition comprises an antibody or antigen-binding fragment that binds to human muscle-specific tyrosine-protein kinase (MuSK) that comprises:
a) A full length heavy chain comprising an amino acid sequence that is at least 80% identical or similar to SEQ ID NO: 270 and
b) A full length light chain comprising an amino acid sequence that is at least 80% identical or similar to SEQ ID NO: 271, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the composition comprises an antibody or antigen-binding fragment that binds to human muscle-specific tyrosine-protein kinase (MuSK) that comprises:
a) A full length heavy chain comprising SEQ ID NO: 270 and
b) A full length light chain comprising SEQ ID NO: 271, and
c) Wherein the full length heavy chain comprises L234A and L235A mutations numbered according the EU numbering system.

In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

The MuSK antibody based molecules of the present invention can be administered by parenteral, topical, oral or intranasal means for therapeutic treatment. Intramuscular injection (for example, into the arm or leg muscles) and intravenous infusion are preferred methods of administration of the molecules of the present invention. In some methods, such molecules are administered as a sustained release composition or device, such as a Medipad™ device (Elan Pharm. Technologies, Dublin, Ireland). In some methods, the antibodies disclosed herein are injected directly into a particular tissue, for example intracranial injection.

In one embodiment, a pharmaceutical composition of the present invention is administered parenterally. The phrases “parenteral administration” and “administered parenterally” as used herein denote modes of administration other than enteral and topical administration, usually by injection, and include epidermal, intravenous, intramuscular, intraarterial, intrathecal, intracapsular, intracranial, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection, subcutaneous and infusion. In one embodiment that pharmaceutical composition is administered by intravenous or subcutaneous injection or infusion.

In an embodiment, an anti-MuSK antibody or antigen binding fragment thereof (or a polynucleotide, an expression vector, a host cell, or a composition) for use according to the invention is administered in combination with an anticholinergic compound. An anticholinergic compound is a compound that is able to inhibit the effect of the neurotransmitter acetylcholine at synapses or at neuroeffector junctions such as neuromuscular junctions. Preferably, an anticholinergic compound is a compound that is able to dampen muscarinic acetylcholine receptor activity.

The anticholinergic compound may also be formulated in a composition as the anti-MuSK antibody or antigen-binding fragment. The type of compositions disclosed herein for the anti-MuSK antibody or antigen-binding fragment may also be used for a composition comprising the anticholinergic compound. The two compounds may be present in a single composition. Alternatively, they may be formulated in separate compositions.

The use of a compound results in activating, inducing a mechanism that promotes NMJ (neuromuscular junction) stability and/or repair is attractive for the treatment of any neuromuscular disease, especially wherein such NMJ is affected. In a preferred embodiment, the use of two different compounds, each activating, inducing a mechanism that promotes NMJ stability and/or repair is even more attractive as it is demonstrated that such combined treatments is synergistic. Therefore, said combination is highly beneficial for the treatment of a neuromuscular disease, especially a neuromuscular disease or disorder with affected NMJ such as ALS.

In an embodiment, the anti-MuSK antibody or antigen binding fragment thereof, polynucleotide, expression vector, host cell, or composition for use according to any of the preceding claims, wherein the neuromuscular disorder is characterized by an impaired neuromuscular transmission and/or an NMJ denervation.

An impaired neuromuscular transmission may be characterized by at least one of:
a. muscarinic overexcitability,
b. motor neuron death,
c. NMJ denervation and
d. impaired synaptic transmission

In an embodiment, an impaired neuromuscular transmission or impaired synaptic transmission may be characterized by a deficient MuSK signaling, deficient MuSK dimerization, deficient MuSK phosphorylation, deficient MuSK signaling and/or deficient acetylcholine receptor clustering.

In an embodiment, an impaired neuromuscular transmission or impaired synaptic transmission may be characterized by a poor motor performance, a decreased grip strength, the poor contractile properties of a muscle at the NMJ, the poor resistance to fatigue of the muscle, a decreased muscle weight.

In an embodiment, a neuromuscular disorder is analyzed or assessed or diagnosed via electrophysiological assessment; pharmacodynamic assessment; the level of neurofilaments (e.g. neurofilament light chain (NFL)) in blood serum, plasma and/or cerebrospinal fluid (CSF); or NMJ biopsies.

A neuromuscular disorder may be selected from the group consisting of: amyotrophic lateral sclerosis (ALS), spinal muscular atrophy (SMA), myasthenia gravis (MG), congenital myasthenia, Lambert-Eaton myasthenic syndrome (LEMS), Lyme disease, poliomyelitis, post-poliomyelitis, heavy metal intoxication, Kennedy syndrome, adult-onset Tay-Sachs disease, hereditary spastic paraplegia, multifocal neuropathy, cervical spondylosis, extramedullary tumor with compressive radiculopathy and myelopathy, inclusion body myositis, progressive bulbar palsy, progressive muscular atrophy, motor neuron syndrome and thyrotoxic myopathy. A preferred neuromuscular disorder is ALS.

In an embodiment, an anti-MuSK antibody or antigen binding fragment thereof as defined herein may be administrated to an asymptomatic ALS subject. It means that such antibody or antigen binding fragment may be administrated prior to the onset of ALS in said subject. The same applies to other neuromuscular disorders.

In this context, an asymptomatic ALS subject may be a subject which has been diagnosed as being predisposed to develop a neuromuscular disorder or disease as ALS. Identifying an individual (or subject) with a neuromuscular disorder may mean that the identification is carried out using a diagnostic method. Such subject may be a symptomatic subject diagnosed at disease onset or after disease onset, or predisposed to develop a neuromuscular disorder or disease (i.e. an asymptomatic subject diagnosed prior to disease onset which is synonymous with disease pre-onset).

A neuromuscular disorder may be caused by a genetic defect. A genetic defect is caused in whole or in part by a change in the genomic DNA sequence relative to the genomic DNA sequence of a corresponding individual or subject not suffering from said genetic defect. A genetic defect can be caused by a mutation in one gene (monogenic disorder), by mutations in multiple genes (multifactorial inheritance disorder), by a combination of gene mutations and environmental factors, or by damage to chromosomes (changes in the number or structure of entire chromosomes, the structures that carry genes). Types of genetic mutation include base substitutions, deletions and insertions.

In an embodiment, the human subject is identified as having (or as being predisposed to develop) a neuromuscular disease caused by genetic defect. In one embodiment, the neuromuscular disease is ALS, and the genetic defect is in the SOD1 gene. Individuals or human subjects predisposed to develop ALS include those having one or more risk factors for developing ALS, including, growing older, having a personal or family history, or a genetic predisposition of one or more SOD-1 associated diseases. One underlying genetic cause or predisposition for ALS is a mutation(s) in the human SOD1 gene. Accordingly, identification of a subject suffering from or susceptible to or predisposed to develop ALS can be performed by genetic testing of the subject's SOD1 gene using assays known in the art, such as e.g., genetic sequencing. At least 180 mutations in human SOD1 are known in the art to be linked to ALS. In an embodiment, the SOD1 mutation is one or more of the mutations selected from the group consisting of: A4V, H46R, G93S, A4T, G141X, D133A, V148G, N139K, G85R, G93A, V14G, C6S, 1113T, D49K, G37R, A89V, E100G, D90A, T137A, E100K, G41A, G41D, G41S, G13R, G72S, L8V, F20C, Q22L, H48R, T54R, S591, V87A, T88deltaTAD, A89T, V97M, S105deltaSL, VI 18L, D124G, LI 14F, D90A, G12R G147R and G37R. In one embodiment, the mutation in the SOD1 gene is G37R.

Accordingly an asymptomatic individual or subject may be identified (prior to disease onset) when said subject has one SOD1 mutation selected from the group consisting of: A4V, H46R, G93S, A4T, G141X, D133A, V148G, N139K, G85R, G93A, V14G, C6S, 1113T, D49K, G37R, A89V, E100G, D90A, T137A, E100K, G41A, G41D, G41S, G13R, G72S, L8V, F20C, Q22L, H48R, T54R, S591, V87A, T88deltaTAD, A89T, V97M, S105deltaSL, VI 18L, D124G, LI 14F, D90A, G12R G147R and G37R.

Analysis of a subject's susceptibility to ALS disease (i.e. asymptomatic subject susceptible to develop ALS) may be performed by analyzing the family history of the subject for ALS. Analysis of the family history may include a three-generation pedigree documenting ALS, a review of medical records and autopsy studies of family members, and identification of an autosomal dominant pattern of SOD1 mutation.

Identification of an individual or subject asymptomatic for ALS (but predisposed to develop such disease) may also be analyzed by an ALS marker. For example, an ALS specific marker may be circulating micro-RNAs, circular RNAs (circRNAs) or messenger RNAs (mRNAs), TDP-42 aggregates, 8-oxo-deoxyguanosine (8-oxodG), 15-F2t-isoprostane (IsoP), plasma TNF-α, IL-10, TRAIL, plasma IL-1b, CSF TRAIL, pro-inflammatory T-helper (Th)17 cells, Th1 cells, anti-inflammatory Th2, regulatory T cells (Treg), pro-inflammatory IL-1b, IL-6, IFN-g, anti-inflammatory IL-10, cholesterol, LDL-cholesterol, apolipoprotein B, HDL-cholesterol, apolipoprotein-AI, plasma creatinine (PCr), plasma ferritin, transferrin, hepcidin, chitotriosidase-1 (CHIT1), chitinase-3-like protein 2 (CHI3L2/YKL39), total tau (tTau), phosphorylated tau (pTau), amyloidb (Ab), novel INHAT repressor (NIR), ubiquitin C-terminal hydrolase-L1 (UCHL1), microtubule-associated protein 2, capping actin protein, gelsolin-like (CAPG), or glycoprotein nonmetastatic melanoma protein B (GPNMB). The human subject may be considered susceptible to ALS disease, when at least one of such marker measurements deviates at least 10%, 20%, 30%, 40%, 50%, 60%, 70%, 80%, 90% from a normal human subject at the same age but without ALS. Analysis of a subject's susceptibility to ALS disease may also be analyzed by imaging. For example, such imaging analysis may be an MRI assessment of skeletal muscle, imaging-derived functional muscle scores, or tongue ultrasound predicted bulbar progression combined with or without MRI.

In an embodiment, the anticholinergic compound is administered separately, sequentially, or concurrently with the anti-MuSK antibody or antigen binding fragment thereof, polynucleotide, expression vector, host cell, cell-free expression system or composition.

In an embodiment, the anticholinergic compound is administered at disease onset or within 1, 2, 3, 4, 5, 6, 7, 8, 9, 10, 11, 12, 13, 14, 15, 16, 17, 18, 19, 20, 21 days or within 1, 2, 3, 4, 5, 6, 7, weeks; or within 1, 2, 3, 4, 5, 6, 7, 8, 9, 10, 11, or 12 months following disease onset. In an embodiment, the anticholinergic compound is administered at disease onset or within one week after disease onset. Surprisingly, attractive results were obtained when the anticholinergic compound was administered at disease onset or as soon as possible after disease onset. It is to be understood by the skilled person that the anticholinergic compound is preferably not used to reduce, diminish a symptom associated with the neuromuscular disorder (such as ALS). In an embodiment, the anticholinergic compound is not used to reduce, diminish urinary urgency. In an embodiment, the anticholinergic compound is not used to reduce or diminish urinary urgency in a neuromuscular disease such as ALS.

In an embodiment, the anticholinergic compound is administered at disease onset, or within 1, 2, 3, 4, 5, 6, 7, 8, 9, 10, 11, 12, 13, 14, 15, 16, 17, 18, 19, 20, or 21 days; within 1, 2, 3, 4, 5, 6, or 7 weeks; or within 1, 2, 3, 4, 5, 6, 7, 8, 9, 10, 11, or 12 months following disease onset, but prior to a diagnosis of the disease. In preferred embodiments, the administration is between diagnosis and 1, 2, 3, 4, 5, 6, 7, 8, 9, 10, 11, 12, 13, 14, 15, 16, 17, 18, 19, 20, or 21 days; within 1, 2, 3, 4, 5, 6, or 7 weeks; or within 1, 2, 3, 4, 5, 6, 7, 8, 9, 10, 11, or 12 months prior to diagnosis.

In an embodiment the anti-MuSK antibody or antigen binding fragment thereof, polynucleotide, expression vector, host cell, or composition is administered pre-onset of the disease or at disease onset. Pre-onset of the disease may mean 1, 2, 3, 4, 5, 6, 7, 8, 9, 10, 11, 12, 13, 14, 15, 16, 17, 18, 19, 20, 21 days pre-onset of the disease or 1, 2, 3, 4, 5, 6, 7, 8 weeks pre-disease onset or 1, 2, 3, 4, 5, 6, 7, 8, 9, 10, 11, 12, 13, 14, 15, 16, 17, 18, 19, 20 months pre-disease onset. In this context, a human subject at pre-onset may mean the human subject is asymptomatic for said neuromuscular disorder such as ALS.

Accordingly, in another aspect of this invention, there is provided an anti-MuSK antibody or antigen binding fragment thereof (polynucleotide, expression vector, host cell, cell-free expression system or composition) for use in the treatment of ALS in a human subject wherein said antibody or antigen binding fragment is administered at pre-onset of the disease, preferably within 1, 2, 3, 4, 5, or 6 months prior to the onset of the disease. In this context, a human subject at pre-onset may mean the human subject is asymptomatic for ALS. In an embodiment, the subject is diagnosed as being predisposed to develop a neuromuscular disorder or disease, such as ALS.

In an embodiment, the antibody or antigen binding fragment binds the MuSK Frizzled (Fz)-like domain sequence of SEQ ID NO: 129.

In an embodiment, the antibody or antigen binding fragment thereof comprises wild-type human IgG constant Fc region comprising at least 80% sequence identity to SEQ ID NO: 266 or 267.

In an embodiment, the antibody or antigen binding fragment is an agonist MuSK antibody and/or has reduced or eliminated effector function.

In an embodiment, the reduced or eliminated effector function is obtained by introducing one or more of the following mutations (all numbered according to the EU numbering system) into the human IgG constant Fc region SEQ ID NO: 266 or SEQ ID NO: 267 of the antibody-based molecule described herein: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In a preferred embodiment, L234A or L235A substitution is introduced into the human IgG constant Fc region of the antibody-based molecule described herein. In a more preferred embodiment, L234A and L235A substitutions are introduced into the human IgG constant Fc region of the antibody-based molecule described herein. This embodiment results in an antibody-based molecule with a heavy chain represented by SEQ ID NO:268 or 270.

In an embodiment, the antibody or antigen binding fragment comprises:
a) a heavy chain variable domain (VH) comprising an amino acid sequence that is at least 80% identical or similar to SEQ ID NO: 234 and
b) a light chain variable domain (VL) comprising an amino acid sequence that is at least 80% identical or similar to SEQ ID NO: 235.

In an embodiment, the antibody or antigen binding fragment comprises a heavy chain variable domain (VH) and a light chain variable domain (VL):
wherein the VH comprises:
   a CDR-H1 amino acid sequence which comprises SEQ ID NO:147 or has 1, 2, 3, 4 or 5 amino acid alterations relative to SEQ ID NO: 147,
   a CDR-H2 amino acid sequence which comprises SEQ ID NO: 153 or has 1, 2, 3, 4 or 5 amino acid alterations relative to SEQ ID NO: 153, and
   a CDR-H3 amino acid sequence which comprises SEQ ID NO: 156 or has 1, 2, 3, 4 or 5 amino acid alterations relative to SEQ ID NO:156 (3B2g2m1) and
wherein the VL comprises:
   a CDR-L1 amino acid sequence which comprises SEQ ID NO: 159 or has 1, 2, 3, 4 or 5 amino acid alterations relative to SEQ ID NO: 159,
   a CDR-L2 amino acid sequence which comprises SEQ ID NO: 172 or has 1, 2, 3, 4 or 5 amino acid alterations relative to SEQ ID NO: 172, and
   a CDR-L3 amino acid sequence which comprises SEQ ID NO: 195 or has 1, 2, 3, 4 or 5 amino acid alterations relative to SEQ ID NO:195 (3B2g2m1).

In an embodiment, the antibody or antigen binding fragment comprises a heavy chain variable domain (VH) and a light chain variable domain (VL):
wherein the VH comprises an amino acid sequence that is at least 80% identical or similar to SEQ ID NO: 234 and the VL comprises an amino acid sequence that is at least 80% identical or similar to SEQ ID NO: 235, and
wherein the VH comprises:
a CDR-H1 amino acid sequence which comprises SEQ ID NO: 147 or has 1, 2, 3, 4 or 5 amino acid alterations relative to SEQ ID NO: 147,
a CDR-H2 amino acid sequence which comprises SEQ ID NO: 153 or has 1, 2, 3, 4 or 5 amino acid alterations relative to SEQ ID NO: 153, and
a CDR-H3 amino acid sequence which comprises SEQ ID NO: 156 or has 1, 2, 3, 4 or 5 amino acid alterations relative to SEQ ID NO:156 (3B2g2m1) and
wherein the VL comprises:
a CDR-L1 amino acid sequence which comprises SEQ ID NO: 159 or has 1, 2, 3, 4 or 5 amino acid alterations relative to SEQ ID NO: 159,
a CDR-L2 amino acid sequence which comprises SEQ ID NO: 172 or has 1, 2, 3, 4 or 5 amino acid alterations relative to SEQ ID NO: 172, and
a CDR-L3 amino acid sequence which comprises SEQ ID NO: 195 or has 1, 2, 3, 4 or 5 amino acid alterations relative to SEQ ID NO:195 (3B2g2m1).

In an embodiment, the antibody or antigen binding fragment comprises:
a) a heavy chain variable domain (VH) comprising an amino acid sequence that is at least 80% identical or similar to SEQ ID NO: 234 and
b) a light chain variable domain (VL) comprising an amino acid sequence that is at least 80% identical or similar to SEQ ID NO: 235.

In an embodiment, the antibody or antigen binding fragment comprises:
a) A full length heavy chain comprising SEQ ID NO: 268 and
b) A full length light chain comprising SEQ ID NO: 269, and
c) Wherein the full length heavy chain comprises L234A and L235A mutations numbered according the EU numbering system.

Surprisingly, attractive results were obtained when the anti-MuSK antibody (or antigen binding fragment or polynucleotide or expression vector of host cell) was administered pre-disease onset. In an embodiment, the human subject is therefore asymptomatic for said neuromuscular disorder such as ALS. In such embodiment, the human subject has been diagnosed as susceptible to develop a neuromuscular disorder as ALS in view of his/her familial history, genetic background or in view of an increased level of neurofilaments (e.g. neurofilament light chain (NFL)) as determined in his/her blood serum or in his/her cerebrospinal fluid (CSF), or in view of a positive genetic test for ALS associated genetic mutation(s), or in view of a change in the level of biomarkers for ALS, or combinations thereof. However he/she does not yet have developed any visible symptoms; he/she is asymptomatic. In a preferred embodiment, the human subject is administered as early as possible after being diagnosed with the genetic defect or ALS associated genetic mutation(s) but not yet having developed any visible symptoms (i.e. asymptomatic subject). In a more preferred embodiment, the human subject is administered as early as possible after being diagnosed with the genetic defect or ALS associated genetic mutation(s) but not yet having developed any visible symptoms, and said human subject has a familial history of ALS. “Immediately” in this context may mean within 1, 2, 3, 4, 5, 6, 7, 8, 9, 10, 11, 12, 13, 14, 15, 16, 17, 18, 19, 20, 21, 22, 23, 24 hours or within 1, 2, 3, 4, 5, 6, or 7 days, or within 1, 2, 3, or 4 weeks. In this context, such treatment shows limited or no toxicity and/or side effects, or any deleterious effects in the eventuality that the ALS diagnosis is not confirmed.

In another aspect of the invention, there is provided a combination comprising an anti-MuSK antibody or antigen binding fragment thereof described herein and an anticholinergic compound. Said combination is preferably for use in the treatment of a neuromuscular disease in a human subject.

Said neuromuscular disorder is characterized by an impaired neuromuscular transmission and/or an denervation at the NMJ (neuromuscular junction). the neuromuscular disorder is characterized by at least one of:
a. muscarinic overexcitability,
b. motor neuron death,
c. neuromuscular junction (NMJ) denervation and
d. impaired synaptic transmission.

In an embodiment, the neuromuscular disorder is selected from the group consisting of: amyotrophic lateral sclerosis (ALS), spinal muscular atrophy (SMA), myasthenia gravis (MG), congenital myasthenia, Lambert-Eaton myasthenic syndrome (LEMS), Lyme disease, poliomyelitis, post-poliomyelitis, heavy metal intoxication, Kennedy syndrome, adult-onset Tay-Sachs disease, hereditary spastic paraplegia, multifocal neuropathy, cervical spondylosis, extramedullary tumor with compressive radiculopathy and myelopathy, inclusion body myositis, progressive bulbar palsy, progressive muscular atrophy, motor neuron syndrome and thyrotoxic myopathy. In preferred embodiment, the neuromuscular disease is ALS.

In this context, a combination does not require that an anti-MuSK antibody or antigen binding fragment thereof described herein, and an anticholinergic compound are physically present together in one composition.

In an embodiment, the anticholinergic compound is administered separately, sequentially, or concurrently. In an embodiment, the antibody or antigen binding fragment is administered at pre-onset of the disease, or within 1, 2, 3, 4, 5, or 6 months prior to the onset of the disease. In an embodiment, said antibody or antigen binding fragment is administered at pre-onset of the disease, or preferably within 1, 2, 3, 4, 5, or 6 months prior to the onset of the disease and/or wherein the anticholinergic compound is administered at disease onset or within 1, 2, 3, 4, 5, 6, or 7 weeks following disease onset. In this context, a human subject at pre-onset may mean the human subject is asymptomatic for said neuromuscular disorder. In an embodiment, the subject treated with the antibody had been first diagnosed as being predisposed to develop a neuromuscular disorder or disease.

In an embodiment, the anti-MuSK antibody or antigen binding fragment thereof binds the MuSK Frizzled (Fz)-like domain sequence of SEQ ID NO: 129.

In an embodiment, the antibody or antigen binding fragment thereof comprises wild-type human IgG constant Fc region comprising at least 80% sequence identity to SEQ ID NO: 266 or 267.

In an embodiment, the antibody or antigen binding fragment is an agonist MuSK antibody and/or has reduced or eliminated effector function.

In an embodiment, the reduced or eliminated effector function is obtained by introducing one or more of the following mutations (all numbered according to the EU numbering system) into the human IgG constant Fc region SEQ ID NO: 266 or SEQ ID NO: 267 of the antibody-based molecule described herein: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In a preferred embodiment, L234A or L235A substitution is introduced into the human IgG constant Fc region of the antibody-based molecule described herein. In a more preferred embodiment, L234A and L235A substitutions are introduced into the human IgG constant Fc region of the antibody-based molecule described herein. This embodiment results in an antibody-based molecule with a heavy chain represented by SEQ ID NO:268 or 270.

In an embodiment, the antibody or antigen binding fragment comprises:
a) a heavy chain variable domain (VH) comprising an amino acid sequence that is at least 80% identical or similar to SEQ ID NO: 234 and
b) a light chain variable domain (VL) comprising an amino acid sequence that is at least 80% identical or similar to SEQ ID NO: 235.

In an embodiment, the antibody or antigen binding fragment comprises a heavy chain variable domain (VH) and a light chain variable domain (VL):
wherein the VH comprises:
   a CDR-H1 amino acid sequence which comprises SEQ ID NO:147 or has 1, 2, 3, 4 or 5 amino acid alterations relative to SEQ ID NO: 147,
   a CDR-H2 amino acid sequence which comprises SEQ ID NO: 153 or has 1, 2, 3, 4 or 5 amino acid alterations relative to SEQ ID NO: 153, and
   a CDR-H3 amino acid sequence which comprises SEQ ID NO: 156 or has 1, 2, 3, 4 or 5 amino acid alterations relative to SEQ ID NO:156 (3B2g2m1) and
wherein the VL comprises:
   a CDR-L1 amino acid sequence which comprises SEQ ID NO: 159 or has 1, 2, 3, 4 or 5 amino acid alterations relative to SEQ ID NO: 159,
   a CDR-L2 amino acid sequence which comprises SEQ ID NO: 172 or has 1, 2, 3, 4 or 5 amino acid alterations relative to SEQ ID NO: 172, and
   a CDR-L3 amino acid sequence which comprises SEQ ID NO: 195 or has 1, 2, 3, 4 or 5 amino acid alterations relative to SEQ ID NO:195 (3B2g2m1).

In an embodiment, the antibody or antigen binding fragment comprises a heavy chain variable domain (VH) and a light chain variable domain (VL):
wherein the VH comprises an amino acid sequence that is at least 80% identical or similar to SEQ ID NO: 234 and the VL comprises an amino acid sequence that is at least 80% identical or similar to SEQ ID NO: 235, and
wherein the VH comprises:
a CDR-H1 amino acid sequence which comprises SEQ ID NO: 147 or has 1, 2, 3, 4 or 5 amino acid alterations relative to SEQ ID NO: 147,
a CDR-H2 amino acid sequence which comprises SEQ ID NO: 153 or has 1, 2, 3, 4 or 5 amino acid alterations relative to SEQ ID NO: 153, and
a CDR-H3 amino acid sequence which comprises SEQ ID NO: 156 or has 1, 2, 3, 4 or 5 amino acid alterations relative to SEQ ID NO:156 (3B2g2m1) and
wherein the VL comprises:
a CDR-L1 amino acid sequence which comprises SEQ ID NO: 159 or has 1, 2, 3, 4 or 5 amino acid alterations relative to SEQ ID NO: 159,
a CDR-L2 amino acid sequence which comprises SEQ ID NO: 172 or has 1, 2, 3, 4 or 5 amino acid alterations relative to SEQ ID NO: 172, and
a CDR-L3 amino acid sequence which comprises SEQ ID NO: 195 or has 1, 2, 3, 4 or 5 amino acid alterations relative to SEQ ID NO:195 (3B2g2m1).

In an embodiment, the antibody or antigen binding fragment comprises:
a) A full length heavy chain comprising SEQ ID NO: 268 and
b) A full length light chain comprising SEQ ID NO: 269, and
c) Wherein the full length heavy chain comprises L234A and L235A mutations numbered according the EU numbering system.

In an embodiment, the antibody or antigen binding fragment comprises:
a) A full length heavy chain comprising SEQ ID NO: 270 and
b) A full length light chain comprising SEQ ID NO: 271, and
c) Wherein the full length heavy chain comprises L234A and L235A mutations numbered according the EU numbering system.

In an embodiment, the anti-MuSK antibody or antigen fragment thereof (polynucleotide, expression vector, host cell, cell-free expression system or composition) is administered pre-onset of the disease(such as 1, 2, 3, 4, 5, 6, 7, 8, 9, 10, 11, 12, 13, 14, 15, 16, 17, 18, 19, 20, or 21 days pre-onset of the disease or 1, 2, 3, 4, 5, 6, or 7 weeks pre-onset of the disease), and the anticholinergic compound is administered at disease onset (such as within 1, 2, 3, 4, 5, 6, 7, 8, 9, 10, 11, 12, 13, 14, 15, 16, 17, 18, 19, 20, or 21 days or within 1, 2, 3, 4, 5, 6, or 7 weeks following disease onset). In this context, a human subject at pre-onset may mean the human subject is asymptomatic for said neuromuscular disorder such as ALS.

In a preferred embodiment, disease onset includes at least one of the symptoms selected from the group consisting of: muscle twitches, muscle cramps, spasticity, muscle weakness, slurred and/or nasal speech, difficulty chewing or swallowing, dysphagia, dysarthria and dyspnea. In a more preferred embodiment, the disease is ALS and disease onset includes at least one of the symptoms selected from the group consisting of: muscle twitches, muscle cramps, spasticity, muscle weakness, slurred and/or nasal speech, difficulty chewing or swallowing, dysphagia, dysarthria and dyspnea.

Disease onset may be assessed by a physician or veterinarian. In an embodiment, beginning of weight loss is considered as disease onset.

In a preferred embodiment, the neuromuscular disorder is ALS and the anti-MuSK antibody or antigen binding fragment comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the neuromuscular disorder is ALS and the anti-MuSK antibody or antigen binding fragment comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the neuromuscular disorder is ALS and the anti-MuSK antibody or antigen binding fragment comprises wild-type human IgG constant Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the neuromuscular disorder is ALS and the anti-MuSK antibody or antigen binding fragment comprises wild-type human IgG constant Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the neuromuscular disorder is ALS and the anti-MuSK antibody or antigen binding fragment comprises wild-type human IgG constant Fc region wherein L234A and/or L235A substitution(s) numbered according the EU numbering system is(are) introduced into said Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the neuromuscular disorder is ALS and the anti-MuSK antibody or antigen binding fragment comprises wild-type human IgG constant Fc region wherein L234A and/or L235A substitution(s) is(are) numbered according the EU numbering system introduced into said Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the neuromuscular disorder is ALS and the anti-MuSK antibody or antigen binding fragment comprises:
a) A full length heavy chain comprising an amino acid sequence that is at least 80% identical or similar to SEQ ID NO: 268 and
b) A full length light chain comprising an amino acid sequence that is at least 80% identical or similar to SEQ ID NO: 269, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the neuromuscular disorder is ALS and the anti-MuSK antibody or antigen binding fragment comprises:
a) A full length heavy chain comprising SEQ ID NO: 268 and
b) A full length light chain comprising SEQ ID NO: 269, and
c) Wherein the full length heavy chain comprises L234A and L235A mutations numbered according the EU numbering system.

In an embodiment, the neuromuscular disorder is ALS and the anti-MuSK antibody or antigen binding fragment comprises:
a) A full length heavy chain comprising an amino acid sequence that is at least 80% identical or similar to SEQ ID NO: 270 and
b) A full length light chain comprising an amino acid sequence that is at least 80% identical or similar to SEQ ID NO: 271, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the neuromuscular disorder is ALS and the anti-MuSK antibody or antigen binding fragment comprises:
a) A full length heavy chain comprising SEQ ID NO: 270 and
b) A full length light chain comprising SEQ ID NO: 271, and
c) Wherein the full length heavy chain comprises L234A and L235A mutations numbered according the EU numbering system.

In embodiments, the anticholinergic compound is a muscarinic receptor antagonist. A muscarinic receptor, also known as a muscarinic acetylcholine receptor or mAchR, is an acetylcholine receptor that forms a G-protein receptor complex in in the cell membrane of certain neurons and other cells. Muscarinic receptors play several roles in mediating the effect of the neurotransmitter acetylcholine. For example, muscarinic receptor are comprised in pre-synaptic membranes of somatic neurons in neuromuscular junctions, where they are involved in the regulation of acetylcholine release.

Five subtypes of muscarinic receptors, M1-M5, are commonly recognized. This classification originates from their different selectivity towards certain agonists and antagonists. M1, M3 and M5 receptors are coupled with Gq proteins in the cell membrane, while M2 and M4 receptors are coupled with Gi/o proteins in the cell membrane. Without being bound to this theory, genes CHRM1-5 encode for M1-M5 receptors, respectively.

The basal or constitutive activity of a muscarinic receptor is defined as the physical, biological and/or chemical activity of the receptor in the absence of acetylcholine, muscarinic receptor agonists and muscarinic receptor antagonists.

An agonist of a muscarinic receptor, also called a muscarinic receptor agonist, is defined as a compound that increases the physical, biological and/or chemical activity of the receptor when it contacts the receptor. An increased activity means an activity similar to the activity caused by contacting the receptor with acetylcholine.

An antagonist of a muscarinic receptor, also called a muscarinic receptor antagonist, is defined as a muscarinic receptor neutral antagonist or muscarinic receptor negative antagonist.

A muscarinic receptor neutral antagonist is a compound that competes with a muscarinic receptor neutral agonist or with a muscarinic receptor negative antagonist for binding to the receptor, thereby blocking the action of the agonist or the negative antagonist (i.e. increasing or decreasing the activity), while the neutral antagonist does not significantly alter the basal activity of the receptor upon binding alone.

In embodiments, the anticholinergic compound is a muscarinic receptor neutral antagonist.

A muscarinic receptor negative antagonist is a compound that decreases the physical, biological and/or chemical activity of the receptor when it contacts the receptor, even in the absence of a muscarinic receptor agonist. A decreased activity means an activity opposite to the activity caused by contacting the receptor with acetylcholine.

In embodiments, the anticholinergic compound is a muscarinic receptor negative antagonist.

A muscarinic receptor antagonist is defined as selective for one or more muscarinic receptor subtypes M1, M2, M3, M4 and/or M5 if the effect of the antagonist (blocking an agonist, blocking a negative antagonist or decreasing the activity) is only significant upon contacting a muscarinic receptor of the one or more subtypes, while there is significantly less or no effect upon contacting a muscarinic receptor of another subtype. Hence, is a muscarinic receptor antagonist is said to be selective for muscarinic receptor M3, it is understood that significantly less or no effect is obtained upon contacting the antagonist with a muscarinic receptor of subtype M1, M2, M4 or M5. In this context, significantly less may be at least 10-, 20-, 30-, 40-, 50-, 60-, 70-, 80-, 90-, 100-, 200-, 300-, 400-, 500-, 600-, 700-, 800-, 900-, 1000-, 10000-, 100000- or 1000000-fold, or at least 10%, 20%, 30%, 40%, 50%, 60%, 70%, 80%, 90%, 100%, 120%, 140%, 160%, 180%, 200%, 220%, 240%, 260%, 280%, 300%, 320%, 340%, 360%, 380%, 400%, 420%, 440%, 460%, 480%, 500%, 600%, 700%, 800%, 900%, 1000%, 1500%, 2000%, 2500%, 3000%, 3500%, 4000%, 4500%, 5000%, 5500%, 6000%, 6500%, 7000%, 7500%, 8000%, 8500%, 9000%, 9500%, 10000%, 20000%, 30000%, 40000%, 50000%, 60000%, 70000%, 80000%, 90000%, 100000%, 1000000%, 10000000% or 100000000% less.

The activity of a muscarinic receptors, preferably of subtype M1, M3 and M5, may be measured using dynamic Ca2+ imaging. These receptors regulate the level of IP3 which then control the release of Ca2+ from internal stores [7].

In embodiments, the anticholinergic compound is a muscarinic receptor antagonist which is:
selective for muscarinic receptor M1, or
selective for muscarinic receptor M3, or
selective for muscarinic receptor M5, or
selective for muscarinic receptor M1 and muscarinic receptor M3, or
selective for muscarinic receptor M1 and muscarinic receptor M5, or
selective for muscarinic receptor M3 and muscarinic receptor M5, or
selective for muscarinic receptor M1, muscarinic receptor M3, and muscarinic receptor M5.

In embodiments, the anticholinergic compound is a muscarinic receptor antagonist which is:
selective for muscarinic receptor M3, or
selective for muscarinic receptor M1 and muscarinic receptor M3, or
selective for muscarinic receptor M3 and muscarinic receptor M5, or
selective for muscarinic receptor M1, muscarinic receptor M3, and muscarinic receptor M5.

In embodiments, the anticholinergic compound is darifenacin, ipratropium bromide, tiotropium bromide, trospium, glycopyrronium, aclidinium, umeclidinium, solifenacin, dicylomine, fesoterodine, flavoxate, glycopyrrolate, propantheline, 1R,2R,4S,5S,7S)-7-[({4-fluoro-2-(thiophen-2-yl)phenyl}carbamoyl)oxy]-9,9-dimethyl-3-oxa-9-azatricyclo[3.3.1.02,4]nonan-9-ium formate (BS46 in [38]), N-(2-[3-([3R]-1-(cyclohexylmethyl)-3-piperidinyl]methylamino)-3-oxopropyl]amino-2-oxoethyl)-3,3,3-triphenyl-propioamide (J-115311 in [39]), 3,3,3-triphenylpropionamide derivatives with one or two amino acid residues between the triphenylpropionic acid moiety and the piperidinylmethylamine moiety ([40]), OrM3 ([41]) or (3R)-3-[[[(3-fluorophenyl)[(3,4,5-trifluorophenyl)methyl]amino]carbonyl]oxy]-1-[2-oxo-2-(2-thienyl)ethyl]-1-azoniabicyclo[2.2.2]octane bromide (CHF 5407 in [41]). Without being bound to this theory, these compounds can be considered muscarinic receptor antagonists.

In embodiments, the anticholinergic compound is darifenacin, ipratropium bromide, tiotropium bromide, trospium, glycopyrronium, aclidinium, umeclidinium, solifenacin, dicylomine, fesoterodine, flavoxate, glycopyrrolate, or propantheline. Without being bound to this theory, darifenacin, ipratropium bromide and tiotropium bromide can be considered muscarinic receptor antagonists.

In embodiments, the anticholinergic compound is darifenacin, ipratropium bromide, tiotropium bromide ortrospium. Without being bound to this theory, darifenacin, ipratropium bromide and tiotropium bromide can be considered muscarinic receptor antagonists.

In a preferred embodiment, the anticholinergic compound is darifenacin. Darifenacin may be represented by the following structure:

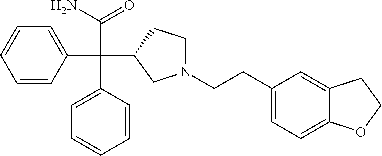 [SMILES: NC(=O)C(c1ccccc1)(c1ccccc1)[C@@H]1CCN(CCc2ccc3c(c2)CCO3)C1]

Preferably, darifenacin is darifenacin hydrobromide. Darifenacin hydrobromide may be represented by the following structure:

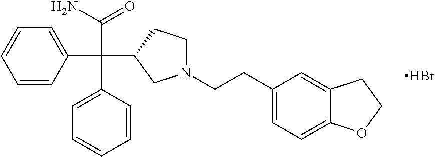 [SMILES: NC(=O)C(c1ccccc1)(c1ccccc1)[C@@H]1CCN(CCc2ccc3c(c2)CCO3)C1 ; Br]

In embodiments, any of the anticholinergic compounds disclosed in the embodiments above may be present as a pharmaceutically acceptable salt thereof. In particular, the anticholinergic compound is darifenacin or a pharmaceutically acceptable salt thereof.

Examples of pharmaceutically acceptable salts include, without limitation, alkali metal (for example, sodium, potassium or lithium) or alkaline earth metals (for example, calcium) salts, however, any salt that is generally non-toxic and effective when administered to the subject being treated is acceptable. Further salts may include, without limitation: (1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, urethane sulfonic acid, ethanesuifonic acid, p-toluenesulfonic acid salicylic acid, tartaric acid citric acid, succinic acid or malonic acid and the like, or (2) salts formed when an acidic proton present in the parent compound either is replaced by a metal ion, e.g., an alkali metal ion, an alkaline earth ion, or an aluminum ion, or coordinates with an organic base such as ethanol amine, diethanolamine, triethanolamine, trimethamine, N-methylglucamine, and the like. Pharmaceutically acceptable salts are well known to those skilled in the art, and any such pharmaceutically acceptable salts may be contemplated in connection with the embodiments described herein.

Acceptable salts may be obtained using standard procedures known in the art, including (without limitation) reacting a sufficiently acidic compound with a suitable base affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, lactate, maiate, maleate, malooate, mesylate, methylsulphate, naphthylate, 2-napsylate, nicotinale, nitrate, orotate, oxalate, palniitate, pamoate, phosphate/hydrogen phosphate/dihydrogen phosphate, saccharate, stearate, succinate, tartrate, tosyiate and trifluoroacetate salts. Suitable base salts of the compounds described herein are formed from bases that form non-toxic salts illustrative, albeit nonlimiting, examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

The anticholinergic compounds disclosed in the embodiments above may be administered as a composition, preferably a therapeutical composition. In embodiments, the composition is formulated as a once-a-day extended release tablet for oral use comprising darifenacin, preferably as darifenacin hydrobromide. Preferably, the compositions comprise one or more of the following excipients: dibasic calcium phosphate anhydrous, hypromellose, magnesium stearate, titanium dioxide, iron oxide yellow, iron oxide red, PEG 400 and/or talc. In an embodiment, the composition is known as ENABLEX™ ENABLEX™ is formulated as a 7.5 mg or 15 mg darifenacin (as darifenacin hydrobromide).

In a preferred embodiment, the neuromuscular disorder is ALS, the anti-MuSK antibody or antigen binding fragment comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1) and an anticholinergic compound is used.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the neuromuscular disorder is ALS, the anti-MuSK antibody or antigen binding fragment comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1) and an anticholinergic compound is used.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the neuromuscular disorder is ALS and the anti-MuSK antibody or antigen binding fragment comprises wild-type human IgG constant Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In a more preferred embodiment, the neuromuscular disorder is ALS and the anti-MuSK antibody or antigen binding fragment comprises wild-type human IgG constant Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the neuromuscular disorder is ALS and the anti-MuSK antibody or antigen binding fragment comprises wild-type human IgG constant Fc region wherein L234A and/or L235A substitution(s) is(are) numbered according the EU numbering system introduced into said Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and

where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1) and
   where the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 195 or having 1, 2, 3, 4 or 5 amino acid alterations relative to SEQ ID NO:195 (3B2g2m1).

In a more preferred embodiment, the neuromuscular disorder is ALS and the anti-MuSK antibody or antigen binding fragment comprises wild-type human IgG constant Fc region wherein L234A and/or L235A substitution(s) numbered according the EU numbering system is(are) introduced into said Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises
an amino acid sequence that is at least 80% identical or similar to SEQ ID NO: 235, and
   where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1) and
   where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the neuromuscular disorder is ALS and the anti-MuSK antibody or antigen binding fragment comprises:
a) A full length heavy chain comprising an amino acid sequence that is at least 80% identical or similar to SEQ ID NO: 268 and
b) A full length light chain comprising an amino acid sequence that is at least 80% identical or similar to SEQ ID NO: 269, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the neuromuscular disorder is ALS and the anti-MuSK antibody or antigen binding fragment comprises:
a) A full length heavy chain comprising SEQ ID NO: 268 and
b) A full length light chain comprising SEQ ID NO: 269, and
c) Wherein the full length heavy chain comprises L234A and L235A mutations numbered according the EU numbering system.

In an embodiment, the neuromuscular disorder is ALS and the anti-MuSK antibody or antigen binding fragment comprises:
a) A full length heavy chain comprising an amino acid sequence that is at least 80% identical or similar to SEQ ID NO: 270 and
b) A full length light chain comprising an amino acid sequence that is at least 80% identical or similar to SEQ ID NO: 271, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the neuromuscular disorder is ALS and the anti-MuSK antibody or antigen binding fragment comprises:
a) A full length heavy chain comprising SEQ ID NO: 270 and
b) A full length light chain comprising SEQ ID NO: 271, and
c) Wherein the full length heavy chain comprises L234A and L235A mutations numbered according the EU numbering system.

In an embodiment a human subject or a patient who has been diagnosed with a neuromuscular disorder such as one of those disclosed above, the MuSK antibody of the present invention optionally combined with the anticholinergic compound are administered to such patient in an amount sufficient to cure, treat, or at least partially arrest the symptoms of the disease (as adduced by biochemical, histologic and/or behavioral assessment), including its complications and intermediate pathological phenotypes in development of the disease. In some embodiments, the administration of the therapeutic molecules of the present invention reduces or eliminates the neuromuscular disorder.

Effective doses of the provided therapeutic molecules of the present invention (i.e. anti-MuSK antibody or antigen binding fragment thereof and anticholinergic compound)), for the treatment of the above-described conditions may vary depending upon many different factors, including means of administration, target site, physiological state of the patient, other medications administered. Treatment dosages are typically titrated to optimize their safety and efficacy. On any given day that a dosage is given, the dosage of the MuSK antibody based molecules as described herein may range from about 0.0001 to about 100 mg/kg, and more usually from about 0.01 to about 20 mg/kg, of the patient's body weight. For example, dosages can be 1 mg/kg body weight or 10 mg/kg body weight or within the range of 1-10 mg/kg body weight. Exemplary dosages thus include: from about 0.1 to about 10 mg/kg body weight, from about 0.1 to about 5 mg/kg body weight, from about 0.1 to about 2 mg/kg body weight, from about 0.1 to about 1 mg/kg body weight, for instance about 0.15 mg/kg body weight, about 0.2 mg/kg body weight, about 0.5 mg/kg body weight, about 1 mg/kg body weight, about 1.5 mg/kg body weight, about 2 mg/kg body weight, about 5 mg/kg body weight, or about 10 mg/kg body weight

A physician or veterinarian having ordinary skill in the art may readily determine and prescribe the effective amount of the pharmaceutical composition required. For example, the physician or veterinarian could start doses of antibody-based molecule in the pharmaceutical composition at levels lower than that required in order to achieve the desired therapeutic effect and gradually increase the dosage until the desired effect is achieved. In general, a suitable daily dose of a composition of the present invention will be that amount of the compound which is the lowest dose effective to produce a therapeutic effect. Such an effective dose will generally depend upon the factors described above. Administration may e.g. be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible the antibody-based molecule of the present invention to be administered alone, it is preferable to administer the antibody-based molecule as a pharmaceutical composition as described above.

For therapeutic purposes, the MuSK antibody-based molecules (and optionally the anticholinergic compound) of the present invention are usually administered on multiple occasions. Intervals between single dosages (e.g., a bolus or infusion) can be weekly, monthly, or yearly. In some embodiments, the MuSK antibody-based molecules (and optionally the anticholinergic compound) of the present invention are administered to the human subject at least 1, at least 2, at least 3, at least 4, at least 5, at least 6, at least 7, at least 8, at least 9, at least 10 times over the course of four months.

In certain embodiments, the human subject is administered a loading dose or loading doses of the pharmaceutical composition followed by a maintenance dose or maintenance doses. In some instances, three loading doses are administered, wherein the loading doses are separated by two weeks for e.g., on day 1, day 15, and day 29. In some instances, the maintenance doses are administered every 4 weeks beginning 4 weeks after the third loading dose (e.g., for 1 month, 2 months, three months, four months, five months, six months, seven months, eight months, nine months, ten months).

In certain embodiments, the human subject is administered three loading doses of the pharmaceutical composition followed by at least one (e.g., 1, 2, 3, 4, 5, 6, 7, 8, 9, 10, 11, 12) maintenance dose. In some instances, the three loading doses are administered two weeks apart. In some instances, the three loading doses are administered 14 days apart. In some instances, the maintenance dose/doses are administered every 4 weeks beginning 4 weeks after the third loading dose. In some instances, the maintenance dose/doses are administered every month beginning one month after the third loading dose. In some instances, the maintenance dose/doses are administered every 28 days beginning 28 days after the third loading dose.

In some methods, dosage is adjusted to achieve a plasma concentration of 1 ng/mL to 1000 μg/ml, preferably 1-1000 μg/mL, more preferably 25-300 μg/mL. Alternatively, the therapeutic molecules of the present invention can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and non-human antibodies. scFv molecules generally have short serum half-lives.

In another embodiment, a pharmaceutical composition comprising a recombinant nucleic acid sequence encoding the MuSK antibody-based molecule as described herein (and optionally in combination with an anticholinergic compound), is administered to a subject to facilitate in vivo expression and formation of the antibody-based molecule for the treatment of conditions mediated by reduced signaling and/or phosphorylation of MuSK. Expression vector constructs suitable for use in this embodiment of the invention are described supra.

The polynucleotide compositions can result in the generation of the MuSK antibody-based molecule in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the antibody-based molecule in the subject within at least about 1 day, 2 days, 3 days, 4 days, 5 days, 6 days, 7 days, 8 days, 9 days, or 10 days of administration of the composition to the subject. The composition can result in generation of the antibody-based molecule in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the persistent generation of the antibody-based molecule in the subject. The composition can result in the generation of the antibody-based molecule in the subject for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

The term “treatment” or “treating” as used herein means ameliorating, slowing or reversing the progress or severity of a disease or disorder, or ameliorating, slowing or reversing one or more symptoms or side effects of such disease or disorder. For purposes of this invention, “treatment” or “treating” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state, delay or slowing of the progression a disease or disorder state, amelioration or palliation of a disease or disorder state, and remission of a disease or disorder, whether partial or total, detectable or undetectable.

Accordingly, in an embodiment, anti-MuSK antibody or antigen binding fragment according to the invention or a composition according to the invention is for use in the treatment of a neuromuscular disorder in a human subject, wherein said treatment results in a stabilization of said disorder. The stabilization may be for at least 1, 2, 3, 4, 5, 6, 7, 8, 9, 10, 11 or 12 months or at least 1, 2 or 3 years.

Each of the therapeutic effects further characterized herein could be seen as a stabilization of the disorder.

In an embodiment, the use of an anti-MuSK antibody or antigen-binding fragment (or polynucleotice, expression vector, host cell, composition) exhibits a therapeutic effect on the treated human subject defined herein.

Such a therapeutic effect may be at least one of the effects disclosed below.

By binding to an epitope of MuSK, the anti-MuSK antibody or antigen binding fragment of the invention are able to elicit an agonistic MuSK activity. Within the context of the application “elicit an agonistic MuSK activity” may be replaced by “activate MuSK”. An agonistic MuSK activity or an activation of MuSK may be triggered at the molecular and/or at the cellular level and/or in a more biological complex system as a NMJ, a synapse, a living organism. In the context of the application, an agonistic MuSK activity may be replaced by the triggering of a MuSK-induced signal or by the induction of MuSK activation in a muscle cell at the NMJ. A MuSK-induced signal (or MuSK activation or MuSK activity) may be at least one of the induction of MuSKdimerization, the induction of MuSKtyrosine phosphorylation, the induction or increase of induction of AChRs clustering at the NMJ (or clustering in vitro in myotubes AChR patches), the increase of the number or percentage of fully innervated NMJ, the decrease of the number or percentage of fully denervated NMJ, maintenance of the number or percentage of fully innervated NMJ (disease stabilization/disease progression stabilization), an improvement of the reliability of synaptic transmission, an improvement of motor performance, a prevention/stabilization or even a reduction/decrease of motor neuron death, an extension of the lifespan of a treated subject.

A MuSK-induced signal by the anti-MuSK antibody of the invention may be the induction of MuSK dimerization, which may be assessed by western blotting. In the context of the invention, an agonistic activity of MuSK may have been assessed when the induction of MuSK dimerization is increased of at least 10%, 20%, 30%, 40%, 50%, 60%, 70%, 80%, 90%, or 100% or more in an experiment using the antibody of the invention by comparison with the same experimental setting without any antibody or with a negative control or with a negative control antibody. Alternatively, in the context of the invention, an agonistic activity of MuSK antibody may have been assessed when the induction of MuSK dimerization is the same or about the same (20% less, 10% less or the same or 10% more or 20% more) in an experiment using the antibody of the invention by comparison with the same experimental setting without a positive control antibody. Such a MuSK dimerization may be assessed without agrin. A positive control in the assessment of MuSK dimerization is agrin.

A MuSK-induced signal by the anti-MuSK antibody of the invention may be the induction of MuSK tyrosine phosphorylation and such phosphorylation may be assessed by western blotting using an antibody specific for tyrosine phosphorylation. In the context of the invention, an agonistic activity of MuSK may have been assessed when the induction of MuSK tyrosine phosphorylation is increased of at least 10%, 20%, 30%, 40%, 50%, 60%, 70%, 80%, 90%, 100%, 120%, 150%, 180%, 200% or more in an experiment using the antibody of the invention by comparison with the same experimental setting without any antibody. Alternatively, in the context of the invention, an agonistic activity of MuSK may have been assessed when the induction of MuSK tyrosine phosphorylation is the same or about the same (20% less, 10% less or the same or 10% more or 20% more) in an experiment using the antibody of the invention by comparison with the same experimental setting without a positive control antibody. Such a MuSK tyrosine phosphorylation may be assessed without agrin. A positive control in the assessment of MuSK tyrosine phosphorylation is agrin.

A MuSK-induced signal by the annti-MuSK antibody of the invention may be the induction of acetylcholine receptor (AChR) clustering at the NMJ and such clustering may be assessed by staining of AChR using an antibody specifically binding to AChR and visualising such staining in fluorescent microscopy using techniques known to the skilled person. Alternatively, the clustering may be assessed in vitro in myotubes AChR patches. A preferred antibody used to visualise AChR clustering is an antibody specific for AChR. More preferred antibody is AlexaFluor488 conjugated α-bungarotoxin (B13422, ThermoFisher). Usually the region to be analysed in fixed in paraformaldehyde and incubated at room temperature with the relevant antibody of the invention or with a positive or negative control and subsequently each region is washed with PBS and observed under an epi-fluorescent microscopy. In the context of the invention, an agonistic activity of MuSK may have been assessed when the induction of AChR clustering at the NMJ is the same or about the same (i.e. 20% less, 10% less or the same or 10% more or 20% more) or is increased of at least 10%, 20%, 30%, 40%, 50%, 60%, 70%, 80%, 90%, or 100% in an experiment using the antibody of the invention by comparison with the same experimental setting without any antibody. Such a AchR clustering may be assessed without agrin. A positive control in the assessment of AchR clustering is agrin.

In a preferred embodiment, the anti-MuSK antibody of the invention exhibits an induction or increase of induction of acetylcholine receptor clustering at the NMJ and such clustering may be assessed by visualizing a staining or an increased staining for AchRs at the NMJ of diaphragms of mice compared to the staining obtained without MuSK agonist antibody. In an embodiment, this induction or increase of clustering of AchRs at the NMJ results in a more normal/physiological NMJ morphology maintaining synaptic innervation and/or pre- and post-synaptic alignment.

A MuSK-induced signal by the anti-MuSK antibody of the invention in a muscle cell at the NMJ may be the increase of the number or percentage of fully innervated NMJ, the decrease of the number or percentage of fully denervated NMJ, maintenance of the number or percentage of fully innervated NMJ (disease stabilization/disease progression stabilization), an improvement of the reliability of synaptic transmission, a prevention/stabilization or even a reduction/decrease of motor neuron death. Each of these features could be assessed using techniques known to the skilled person such as staining of AchR using the α-bungarotoxin antibody as earlier defined herein, presynaptic labelling and quantifying innervation by fluorescent confocal microscopy, EMG single fibre EMG, electrophysiology of single synapses, staining of motor neuron cell bodies in bone marrow specific regions. All these assays have been described in Cantor S et al 2018 (Elife, 2018; 7:e34375).

An anti-MuSK antibody or antigen-binding fragment may improve the motor performance and/or grip strength of the treated subject. The motor performance and grip strength of a treated subject may have been considered to have been improved when such motor perfromance or grip strength may have been increased of at least 10%, 20%, 30%, 40%, 50%, 60%, 70%, 80%, 90%, or 100% in an experiment using the anti-MuSK antibody of the invention by comparison with the same experimental setting without any antibody. The motor performance (or grip strength) of a treated subject may be assessed using assays known to the skilled person. The experimental part discloses some exemplary methods.

An anti-MuSK antibody or antigen-binding fragment may improve the contractile properties of a muscle at the NMJ of the treated subject. The contractile properties of a muscle of a treated subject may have been considered to have been improved when such contractile properties may have been increased of at least 10%, 20%, 30%, 40%, 50%, 60%, 70%, 80%, 90%, or 100% in an experiment using the anti-MuSK antibody of the invention by comparison with the same experimental setting without any antibody. The contractile properties of the muscle of a treated subject (at the NMJ) may be assessed using assays known to the skilled person. The experimental part discloses some exemplary methods. In this context, the subject may be an animal.

An anti-MuSK antibody or antigen-binding fragment may improve the resistance to fatigue of a muscle at the NMJ of the treated subject. The resistance to fatigure of a muscle of a treated subject may have been considered to have been improved when such fatigue properties may have been improved of at least 10%, 20%, 30%, 40%, 50%, 60%, 70%, 80%, 90%, or 100% in an experiment using the anti-MuSK antibody of the invention by comparison with the same experimental setting without any antibody. The fatigure properties of the muscle of a treated subject (at the NMJ) may be assessed using assays known to the skilled person. The experimental part discloses some exemplary methods. In this context, the subject may be an animal.

An anti-MuSK antibody or antigen-binding fragment may induce an increase of the muscle weight at the NMJ of the treated subject. The muscle weight of a treated subject may have been considered to have been improved when such weight may have been increased of at least 10%, 20%, 30%, 40%, 50%, 60%, 70%, 80%, 90%, or 100% in an experiment using the anti-MuSK antibody of the invention by comparison with the same experimental setting without any antibody. The experimental part discloses some exemplary methods. In this context, the subject may be an animal.

A MuSK-induced signal or effect by the anti-MuSK antibody of the invention may be characterized by the improvement of the quality of life or the delay in the apparition of the deterioration of the quality of life of a treated subject. The quality of life may be quantify by the weight of the subject. The improvement of the quality of life or the delay in the apparition of the deterioration of the quality of life may be of at least 1 day, 1 week, 2 weeks, 3 weeks, 4 weeks, 1 month, 2 months, 3 months, 4 months, 5 months, 6 months, 7 months, 8 months, 9 months, 10 months, 11 months, 1 year or more. This is assessed in comparison with the expected quality of life (or the expected apparition of the deterioration of the quality of life) of a subject suffering from the same condition and having not been treated with an antibody of the invention. In this context, the subject may be an animal.

A MuSK-induced signal or effect by the anti-MuSK antibody of the invention may be characterized by the lifespan of a treated subject. The extension may be of at least 1 day, 1 week, 2 weeks, 3 weeks, 4 weeks, 1 month, 2 months, 3 months, 4 months, 5 months, 6 months, 7 months, 8 months, 9 months, months, 11 months, 1 year or more. This is assessed in comparison with the expected lifespan of a subject suffering from the same condition and having not been treated with an antibody of the invention. In this context, the subject may be an animal.

The properties of the anti-MuSK antibody described herein may be measured in accordance with the assays described herein. An activating activity of a MuSK agonist antibody may be measured relative to a control, for example a negative control antibody (such as an isotype control) that may not bind MuSK. A preferred control antibody not binding to MuSK is Motavizumab which targets RSV (Review, MAbs, 1(5), 439-442, September-Octo 2009, DOI: 10.4161/mabs.1.5.9496). A preferred positive control agonist MuSK antibody is mAb #13 from Genentech. Another preferred positive control molecule for evidencing an activating MuSK activity is agrin (rat agrin from R&D systems, 550-AG).

In another embodiment, the anti-MuSK antibody or antigen-binding fragment thereof (or polynucleotide, expression vector, host cell, composition) combined with an anticholinergic compound as defined earlier herein exhibit a therapeutic effect in the treated human subject defined herein. In a preferred embodiment, additional and more preferably synergistic therapeutic effects are elicited when both compounds are used compared to the use of the anti-MuSK antibody or antigen-binding fragment (or polynucleotide, expression vector, host cell, composition) as stand alone therapy.

Additional therapeutic effects may be the reduction (‘dampening’) of the muscarinic activity of perisynaptic Schwann cells (PSC), the NMJ repair. Such additional therapeutic effects may be the specific reduction (‘dampening’) of the muscarinic activity of PSC. Such additional therapeutic effects may be the reduction (‘dampening’) of the hyperexcitability of PSC in the context of a neuromuscular discorder. The compound or combination of the present invention specifically acts on the muscarinic receptor. The compound or combination of the present invention does not seem to have any effect on the purigenic receptor expressed on PSC.

NMJ repair may be the induction or increase of nerve sprouting and/or the increase of the innervation status of the NMJ. Each of these effects may be assessed using techniques known to the skilled person. Also, dampening the muscarinic activity of PSCs may help to maintain NMJ innervation.

In the context of the invention, an induction or increase of nerve sprouting (or of the innervation status of the NMJ) may have been assessed when the induction of nerve sprouting at the NMJ (or of the innervation status of the NMJ) is increased of at least 10%, 20%, 30%, 40%, 50%, 60%, 70%, 80%, 90%, or 100% in an experiment using the anticholinergic compound by comparison with the same experimental setting without said compound. Nerve sprouting or innervation status may be assessed using immunohistochemistry on nerve-muscle preparations. The experimental part discloses how to obtain such nerve-muscle preparations.

In the context of the invention, the reduction of the muscarinic activity of PSC (or the reduction of the muscarinic hyperexcitability or overexcitability) may have been assessed when such activity (or such hyperexcitability or overexcitability) has been reduced of at least 10%, 20%, 30%, 40%, 50%, 60%, 70%, 80%, 90%, or 100% in an experiment using the anticholinergic compound by comparison with the same experimental setting without said compound.

Accordingly, in an embodiment, the use of an anti-MuSK antibody or antigen-binding fragment (or polynucleotide, expression vector, host cell, composition), preferably combined with an anticholinergic compound as defined earlier herein, exhibit one or more of the following therapeutic effects:
an increase of the number or percentage of fully innervated NMJ in the subject, maintenance of the number or percentage of fully innervated NMJ in the subject, the decrease of the number or percentage of fully denervated NMJ in the subject, an improvement of the reliability of synaptic transmission, a prevention, stabilization or reduction of motor neuron death in the subject; and/or
an improvement of the motor performance and/or grip strength of the subject; and/or
an improvement of the contractile properties of a muscle at the NMJ of the subject; and/or
an improvement of the resistance to fatigue of a muscle at the NMJ of the subject; and/or
an induction of an increase of the muscle weight at the NMJ of the subject; and/or
an improvement of the quality of life or the delay in the apparition of the deterioration of the quality of life of the subject; and/or
a reduction of the muscarinic activity (or the reduction of the muscarinic hyperexcitability or overexcitability) of perisynaptic Schwann cells (PSC) in the subject, or of the NMJ repair in the subject.

As demonstrated in the experimental part, synergistic therapeutic effects are obtained when both compounds are used. These synergistic effects include the improvement/increase of the following parameters/symptoms: locomotor function and grip strength, the contractile properties of a muscle at the NMJ, resistance to fatigue of the muscle, muscle weight, impact on the general condition of life such as body weight.

An “effective amount,” of the antibody-based molecule refers to an amount sufficient, at dosages and for periods of time necessary, to achieve an intended biological effect or a desired therapeutic result including, without limitation, clinical results. The phrase “therapeutically effective amount” when applied to an antibody-based molecule of the invention is intended to denote an amount of the antibody that is sufficient to ameliorate, palliate, stabilize, reverse, slow or delay the progression of a disorder or disease state, or of a symptom of the disorder or disease. In an embodiment, the method of the present invention provides for administration of the antibody-based molecule in combinations with other compounds. In such instances, the “effective amount” is the amount of the combination sufficient to cause the intended biological effect.

In a further aspect, there is provided a method for the prevention and/or treatment of a neuromuscular disease and/or disorder and/or condition comprising administering to a subject in need thereof, an anti-MuSK antibody or antigen-binding fragment thereof (a polynucleotide, an expression vector, host cell, or composition all as earlier defined herein) and preferably an anticholinergic compound. All features of this method have been defined earlier herein.

In a further aspect, there is provided a use of an anti-MuSK antibody or antigen-binding fragment thereof (a polynucleotide, an expression vector, host cell, or composition all as earlier defined herein) and preferably an anticholinergic compound for the manufacture of a medicament for the prevention and/or treatment of a neuromuscular disease and/or disorder and/or condition. All features of this use have been defined earlier herein.

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. Each embodiment described herein may be combined together with any other embodiment described herein, unless otherwise indicated.

The present invention is further described by the following examples which should not be construed as limiting the scope of the invention.

### REFERENCES

1. L. R. Fischer et al., Amyotrophic lateral sclerosis is a distal axonopathy: evidence in mice and man. Exp Neurol 185, 232-240 (2004).
2. D. Frey et al., Early and selective loss of neuromuscular synapse subtypes with low sprouting competence in motoneuron diseases. J Neurosci 20, 2534-2542 (2000).
3. E. Martineau, A. Di Polo, C. Vande Velde, R. Robitaille, Dynamic neuromuscular remodeling precedes motor-unit loss in a mouse model of ALS. Elife 7, (2018).
4. S. Pun, A. F. Santos, S. Saxena, L. Xu, P. Caroni, Selective vulnerability and pruning of phasic motoneuron axons in motoneuron disease alleviated by CNTF. Nat Neurosci 9, 408-419 (2006).
5. E. Tremblay, E. Martineau, R. Robitaille, Opposite Synaptic Alterations at the Neuromuscular Junction in an ALS Mouse Model: When Motor Units Matter. J Neurosci 37, 8901-8918 (2017).
6. C. Yang et al., Mutant PFN1 causes ALS phenotypes and progressive motor neuron degeneration in mice by a gain of toxicity. Proc Natl Acad Sci USA 113, E6209-E6218 (2016).
7. D. Arbour, E. Tremblay, E. Martineau, J. P. Julien, R. Robitaille, Early and persistent abnormal decoding by glial cells at the neuromuscular junction in an ALS model. J Neurosci 35, 688-706 (2015).
8. D. Arbour, C. Vande Velde, R. Robitaille, New perspectives on amyotrophic lateral sclerosis: the role of glial cells at the neuromuscular junction. J Physiol 595, 647-661 (2017).
9. C. P. Ko, R. Robitaille, Perisynaptic Schwann Cells at the Neuromuscular Synapse: Adaptable, Multitasking Glial Cells. Cold Spring Harb Perspect Biol 7, a020503 (2015).
10. R. Robitaille, B. S. Jahromi, M. P. Charlton, Muscarinic Ca2+ responses resistant to muscarinic antagonists at perisynaptic Schwann cells of the frog neuromuscular junction. J Physiol 504 (Pt 2), 337-347 (1997).
11. D. Rochon, I. Rousse, R. Robitaille, Synapse-glia interactions at the mammalian neuromuscular junction. J Neurosci 21, 3819-3829 (2001).
12. M. C. Wright et al., Distinct muscarinic acetylcholine receptor subtypes contribute to stability and growth, but not compensatory plasticity, of neuromuscular synapses. J Neurosci 29, 14942-14955 (2009).
13. J. Georgiou, R. Robitaille, M. P. Charlton, Muscarinic control of cytoskeleton in perisynaptic glia.

### J Neurosci 19, 3836-3846 (1999).

14. J. Georgiou, R. Robitaille, W. S. Trimble, M. P. Charlton, Synaptic regulation of glial protein expression in vivo. Neuron 12, 443-455 (1994).
15. J. P. O'Malley, M. T. Waran, R. J. Balice-Gordon, In vivo observations of terminal Schwann cells at normal, denervated, and reinnervated mouse neuromuscular junctions. J Neurobiol 38, 270-286 (1999).
16. P. A. Perez-Gonzalez, Provost, F., Rousse, I., Benzina, O., Piovesana, R., Darabid, H., Lamoureux, B., Wang, Y-S, Arbour, D. and Robitaille, R., Functional adaptation of glial cells at neuromuscular junctions in response to injury. (In revision to GLIA (GLIA-00440-202)).
17. M. L. Reynolds, C. J. Woolf, Terminal Schwann cells elaborate extensive processes following denervation of the motor endplate. J Neurocytol 21, 50-66 (1992).
18. Y. J. Son, W. J. Thompson, Nerve sprouting in muscle is induced and guided by processes extended by Schwann cells. Neuron 14, 133-141 (1995).
19. Y. J. Son, W. J. Thompson, Schwann cell processes guide regeneration of peripheral axons. Neuron 14, 125-132 (1995).
20. E. Martineau, D. Arbour, J. Vallee, R. Robitaille, Properties of Glial Cell at the Neuromuscular Junction Are Incompatible with Synaptic Repair in the SOD1(G37R) ALS Mouse Model. J Neurosci 40, 7759-7777 (2020).
21. S. J. Burden, The formation of neuromuscular synapses. Genes Dev 12, 133-148 (1998).
22. S. Cantor et al., Preserving neuromuscular synapses in ALS by stimulating MuSK with a therapeutic agonist antibody. Elife 7, (2018).
23. M. J. Perez-Garcia, S. J. Burden, Increasing MuSK activity delays denervation and improves motor function in ALS mice. Cell Rep 2, 497-502 (2012).
24. J. Oury et al., Mechanism of disease and therapeutic rescue of Dok7 congenital myasthenia. Nature 595, 404-408 (2021).
25. C. R. Chapple, Darifenacin: a novel M3 muscarinic selective receptor antagonist for the treatment of overactive bladder. Expert Opin Investig Drugs 13, 1493-1500 (2004).
26. F. Haab, Darifenacin in the treatment of overactive bladder. Drugs Today (Barc) 41, 441-452 (2005).
27. C. Chapple et al., A pooled analysis of three phase Ill studies to investigate the efficacy, tolerability and safety of darifenacin, a muscarinic M3 selective receptor antagonist, in the treatment of overactive bladder. BJU Int 95, 993-1001 (2005).
28. F. Haab, L. Stewart, P. Dwyer, Darifenacin, an M3 selective receptor antagonist, is an effective and well-tolerated once-daily treatment for overactive bladder. Eur Urol 45, 420-429; discussion 429 (2004).
29. E. Callegari et al., A comprehensive non-clinical evaluation of the CNS penetration potential of antimuscarinic agents for the treatment of overactive bladder. Br J Clin Pharmacol 72, 235-246 (2011).
30. G. G. Kay, U. Ebinger, Preserving cognitive function for patients with overactive bladder: evidence for a differential effect with darifenacin. Int J Clin Pract 62, 1792-1800 (2008).
31. M. Filali, R. Lalonde, S. Rivest, Sensorimotor and cognitive functions in a SOD1(G37R) transgenic mouse model of amyotrophic lateral sclerosis. Behav Brain Res 225, 215-221 (2011).
32. P. C. Wong et al., An adverse property of a familial ALS-linked SOD1 mutation causes motor neuron disease characterized by vacuolar degeneration of mitochondria. Neuron 14, 1105-1116 (1995).
33. A. C. Ludolph et al., Guidelines for preclinical animal research in ALS/MND: A consensus meeting. Amyotroph Lateral Scler 11, 38-45 (2010).
34. S. Boillee et al., Onset and progression in inherited ALS determined by motor neurons and microglia. Science 312, 1389-1392 (2006).34. Knippenberg, S., et al., Significance of behavioural tests in a transgenic mouse model of amyotrophic lateral sclerosis (ALS). Behav Brain Res, 2010. 213(1): p. 82-7.
35. Rizzuto, E., et al., Measuring Neuromuscular Junction Functionality in the SOD1(G93A) Animal Model of Amyotrophic Lateral Sclerosis. Ann Biomed Eng, 2015. 43(9): p. 2196-206.
36. Dibaj, P., E.D. Schomburg, and H. Steffens, Contractile characteristics ofgastrocnemius-soleus muscle in the SOD1G93A ALS mouse model. Neurol Res, 2015. 37(8): p. 693-702.
37. Kanning, K.C., A. Kaplan, and C.E. Henderson, Motor neuron diversity in development and disease. Annu Rev Neurosci, 2010. 33: p. 409-40.
38. Atkin, J. D., et al., Properties of slow- and fast-twitch muscle fibres in a mouse model of amyotrophic lateral sclerosis. Neuromuscul Disord, 2005. 15(5): p. 377-88.

### EXAMPLES

### Example 1: Methods

### Animals

Mice overexpressing the human mutated SOD1^{G37R} transgene, line 29, were obtained from The Jackson Laboratory and bred at Université de Montreal animal facilities on a C57BL/6 background. This mouse model is a late onset, slowly progressing model of ALS that recapitulates the human phenotype of the disease. Characterization of this strain phenotype has been previously published in several ALS studies (5, 7, 20, 31, 32). All experiments were performed in accordance with the guidelines of the Canadian Council of Animal Care and the Comité de deontologie animale of Université de Montreal.

### Preclinical Trial Design

The pre-clinical trial design was made according to guidelines for preclinical animal research in ALS/MND (33). The study was conducted in a double-blind manner. Fifteen male mice from the SOD1^{G37R} background were randomly assigned to three groups.
1. ARGX-119 (3B2g2m1-hlgG1LALAdelk: full length heavy chain with reduced effector function SEQ ID NO: 268 and full length light chain with reduced effector function SEQ ID: 269):and darifenacin
2. ARGX-119 (3B2g2m1-hlgG1LALAdelk) and Vehicle (darifenacin control)
3. Isotype control mAb+Vehicle (darifenacin control)

ARGX-119 treatment was started at pre-onset (before symptoms or asymptomatic) and darifenacin treatment at disease onset (appearance of symptoms) and continued until sacrifice. A set of neurological scores (level 1 to 5, Appendix 1) was used to determine the onset of symptoms and the progression and severity of symptoms during disease progression. Onset of disease was assessed by the beginning of weight loss (34) and appearance of tremor, representing a neurological score of 1 while the endpoint of this study was at the late symptomatic stage, representing a neurological score of between 3 and 5.

Intra-peritoneal injections of ARGX-119 MuSK antibody (3B2g2m1-hlgG1LALAdelk) or the placebo (Motavizumab-hlgG1LALAdelk: full length heavy chain with reduced effector function SEQ ID NO: 272 and full length light chain with reduced effector function SEQ ID: 273) were initiated at P400 at an initial dose of 20 mg/kg and then weekly at a dose of 10 mg/kg until sacrifice. Darifenacin was given orally (10 mg/kg diluted in DMSO, 5 days/week) initiated at disease onset (˜P425). Placebo for darifenacin was DMSO alone. Mice received both treatments for about 4 months, until the age of ˜520 days, the median age at which they normally reach critical disease endpoints.

Motavizumab-hlgG1 LALAdelk: SEQ ID NO: 272 is derived from SEQ ID NO: 274 and SEQ ID: 273 from SEQ ID:275.

Treatment, behavior monitoring, experiments and results analysis were done blindly. Standard ALS behavioral measurements were performed weekly to measure disease progression in the various study groups. This includes rotarod test, grip strength measurements, weight measurements and tail suspension test to assess hindlimb extension reflex. At the time of the sacrifice, Extensor Digitorum Longus (EDL) and Soleus (SOL) muscles and their innervation were dissected and placed in a physiological chamber. Two sets of measurements were acquired. First, the functional properties of the muscles (strength and fatigue) were determined using a force transducer. Second, muscles were fixed, and the muscle mass were determined.

### Rotarod Acceleration Protocol

Motor coordination, strength and balance were assessed using a rotarod (TSE Rotarod, TSE Systems Gmbh, Germany). Animals were placed onto a rotating wheel at a starting speed of 4 rpm, increasing to 40 rpm in 300 seconds. Mice had two attempts per block and 2 blocks per session (with a rest time between block) to remain on the rotarod during the acceleration protocol, and the two longest latencies to fall were averaged.

### Grip Strength

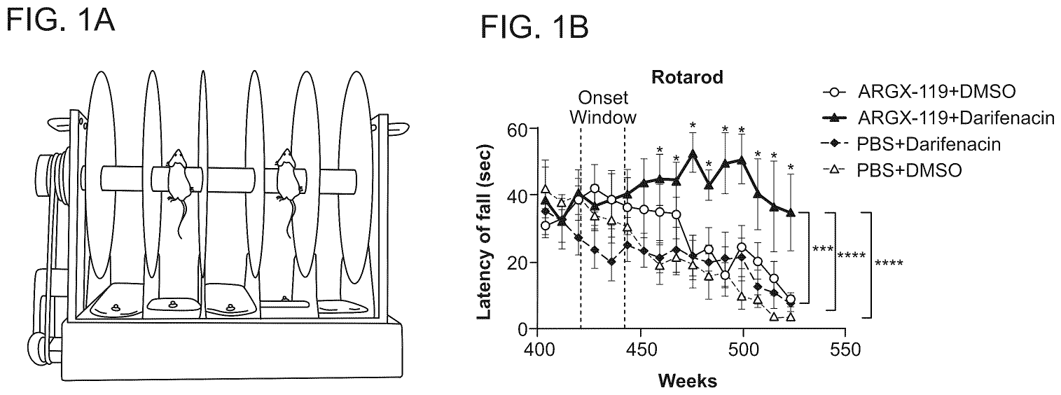

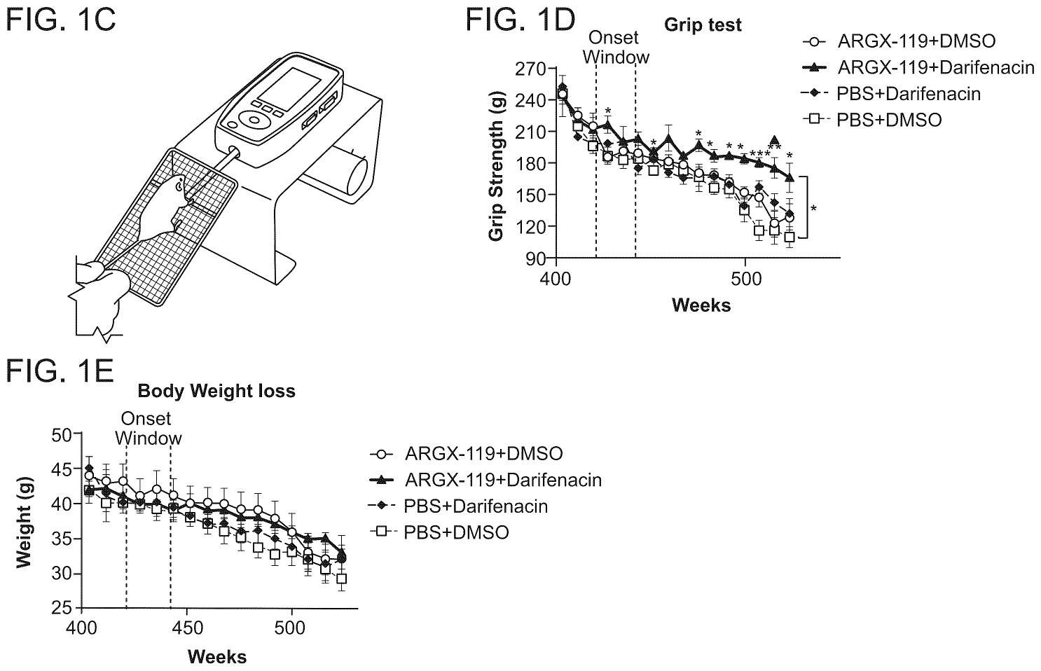

To measure the overall strength of the limbs of mice, a grip strength meter was used (FIG. 1C). Mice had nine attempts per session (3 blocks of 3 attempts, each block was separated by a 1 min rest period) and the best three values of each block were averaged.

### Nerve-Muscle Preparations

Preparations of EDL and SOL muscles and their innervating nerve were dissected in oxygenated Ree's solution (in mM), as follows: 110 NaCl, 5 KCl, 1 MgCl₂, 25 NaHCO₃, 2 CaCl₂, 11 glucose, 0.3 glutamic acid, 0.4 glutamine, 5 BES (N,N-Bis(2-hydroxyethyl)-2-aminoethanesulfonic acid sodium salt), 0.036 choline chloride, and 4.34×10⁻⁷ cocarboxylase. After dissection, nerve muscle preparations were constantly perfused with oxygenated Ree's solution (95% O₂, 5% CO₂).

### Measurements of Neuromuscular Properties

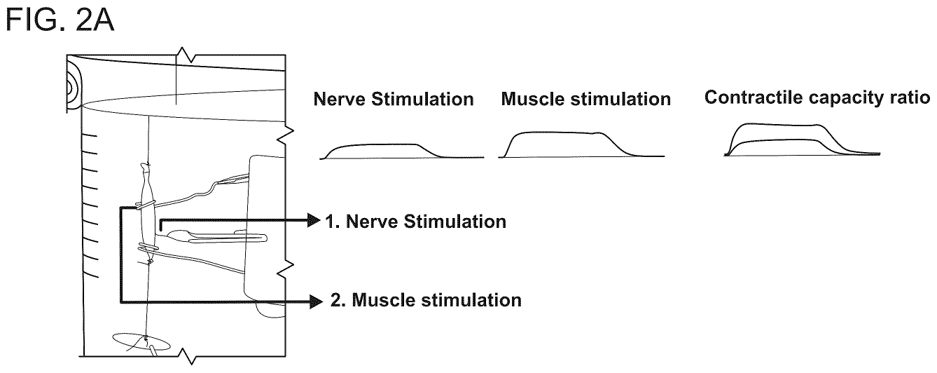

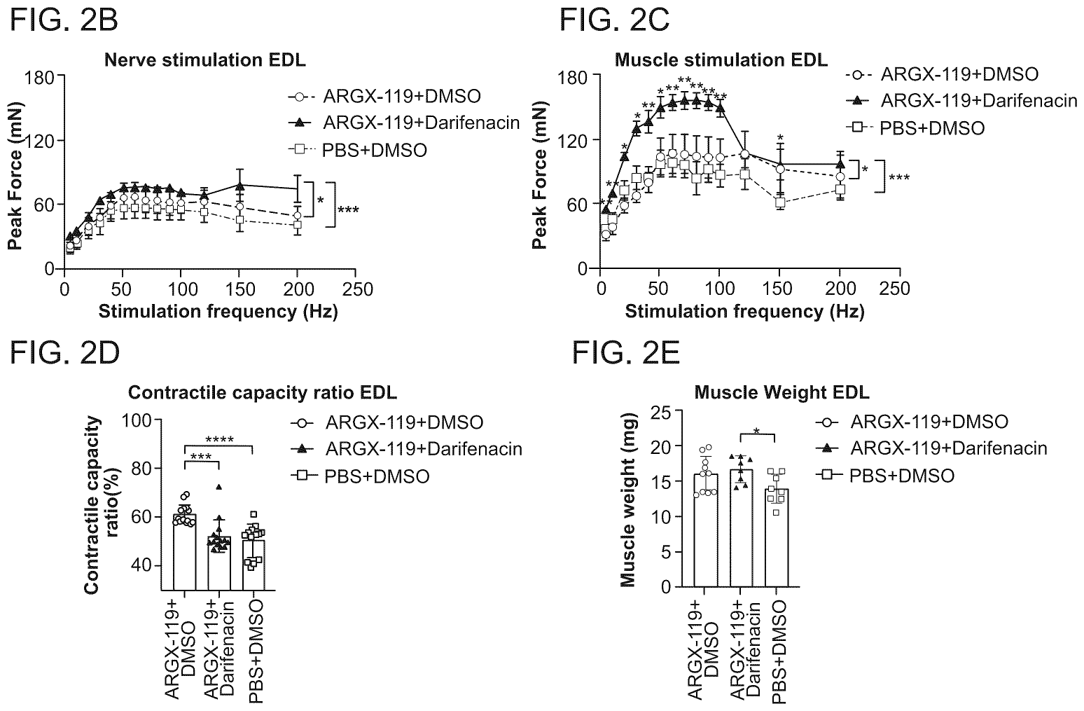

The EDL and SOL nerve-muscle preparations were attached vertically to a fixed force transducer (model 402A-500 mN, Aurora Scientific Inc.) using surgical threads. The preparations were attached at the tendons level to the transducer at one extremity and to an adaptable hook at the opposite extremity (FIG. 2A). A platinum reference electrode was then juxtaposed to the muscle, positioned near an extremity of the muscle, close to a tendon. To stimulate the muscle, a second platinum electrode was juxtaposed at the other extremity of the muscle. To elicit muscle contractions from motor nerve and neuromuscular activity, the tibial nerve (SOL) or the deep peroneal nerve (EDL) was suctioned into an electrode made of PE tubing and filled with physiological solution. Hence, this system was designed to elicit muscle contractions from both muscle and/or nerve stimulations. Neuromuscular contractile basal force responses were elicited by a single supra-maximal square-wave of 500 mV, 0.1 ms pulse imposed on the motor nerve. Muscle contractile basal force responses were elicited by square pulse stimulation of 15 V, 1 ms. Optimal muscle length was determined by gradually stretching the muscle until maximal contractile force output was attained.

Force-frequency curve: Nerve and muscle stimulations were performed to generate a standard force-frequency curve. Alternate nerve and muscle stimulations were performed at various frequencies for 500 ms (5 Hz, 10 Hz, 20 Hz, 30 Hz, 40 Hz, 50 Hz, 60 Hz, 70 Hz, 80 Hz, 90 Hz, 100 Hz, 120 Hz, 140 Hz, 160 Hz, 180 Hz, 200 Hz, 250 Hz and 300 Hz) and the force generated monitored. There was a 2-minute rest period between each stimulation. The proportion of the muscle capacity that is used by the neuromuscular system upon nerve stimulation was expressed as the contractile capacity ratio and calculated as follow for each

$$\frac{{Force}_{Nerve}}{{Force}_{Muscle}} X 100$$

frequency:

Maximal force: The maximal force generated by the alternated nerve and muscle stimulation was obtained at a frequency of 50 Hz and 80 Hz for 2 sec, each separated by either 2 (SOL) or 5 (EDL) minutes.

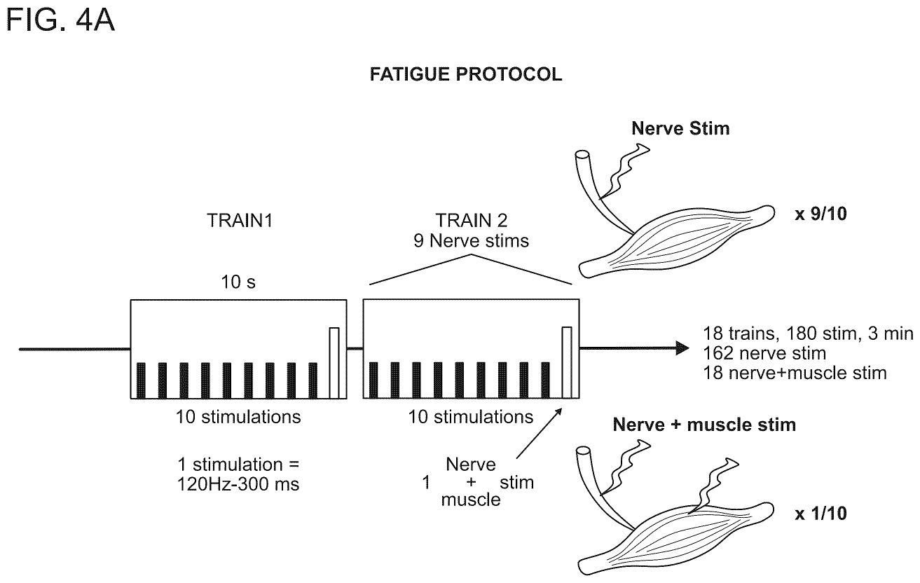

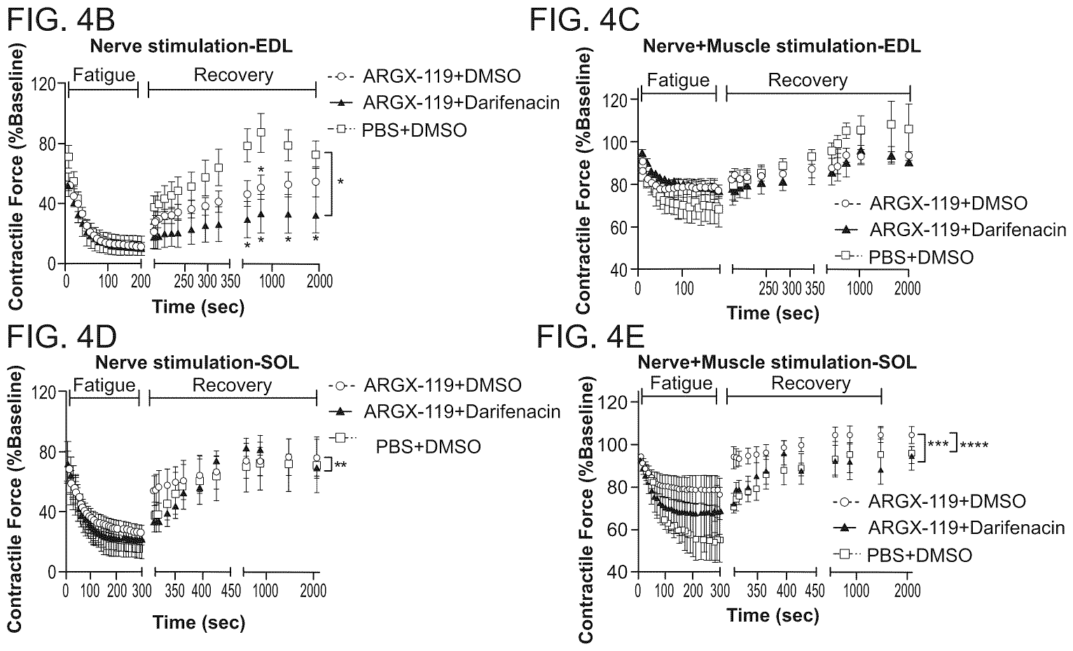

Muscle fatigue: The fatigue protocol is illustrated in FIG. 4A. The fatigue protocol was adapted to each muscle owing to the differences in their intrinsic properties. For the EDL, fatigue was tested using a bout of 180 nerve stimulations for a duration of 300 ms, elicited at a frequency of 120 Hz. The rest period between each stimulation was 700 ms, for a total protocol duration of 3 min. Muscular stimulations were super-imposed to nerve stimulations every 10stimulations (18 simultaneous nerve-muscle stimulations), to evaluate muscular reserve. The fatigue protocol for the SOL consisted of a bout of 300 nerve stimulations for 500 ms at 50 Hz, with a rest period of 600 ms between stimulations, for a total duration of 5 min 30. Muscular stimulations were super-imposed to nerve stimulations every 10 stimulations (30 simultaneous nerve-muscle stimulations).

Muscle recovery: Each fatigue protocol was followed by a 30 min recovery period during which neuromuscular and neuromuscular+muscular contractile force (120 Hz-300 ms for the EDL and 50 Hz-500 ms for the SOL) were measured after the fatigue protocol at 5 s, 10 s, 15 s, 30 s, 45 sec, 1 min, 1.5 min, 2 min, 2.5 min, 5 min, 10 min, 20 min and 30 min.

### Muscle Weight

After each experiment, muscles were fixed (10 min, PFA) and washed (3 washes of 5 min each, PBS 1X). Then, both tendons were cut and muscles were weighted and stored at 4° C. for further processing.

### Statistics

Results are represented as the mean±SEM where the number of animals is identified as N (number of replicates) and the number of muscles is represented by n (number of observations). One-way ANOVA Kruskal-Wallis test and multiple t-test were used in most cases where three or four different groups were compared. Repeated one-way ANOVA with post hoc Bonferroni multiple comparison test was used to compare the values obtained at various frequencies or over time from the same animals in the different groups. The confidence level used in the study was 95% (a=0.05). All analyses were made with GraphPad 8 software (Prism).

### Example 2: Results

ARGX-119 antibody combined with darifenacin improves locomotor function and grip strength The locomotor function and general strength of the animal were tested to investigate if the combination of treatment with ARGX-119 antibody and darifenacin could improve muscle function.

First, the motor performance, balance and coordination, were measured using a standard acceleration protocol on the Rotarod (FIG. 1A), which is known to reveal ALS motor deficits as disease progresses (34). FIG. 1B shows the progressive decline of motor performance of mice of the ARGX-119 alone, darifenacin alone or placebo-treated group as revealed by a shorter latency to fall of the Rotarod, showing the expected progression of ALS motor phenotype. However, the motor performance of mice combo-treated with ARGX-119 antibody and darifenacin mice was much less pronounced, resulting in a significantly improved motor performance compared to the ARGX-119+DMSO (p<0.001), PBS+darifenacin (p<0.0001) and placebo-treated mice (p<0.0001) (FIG. 1B; ARGX-119+DMSO N=5, ARGX-119+darifenacin N=4, PBS+darifenacin N=5, Placebo N=5, One-way ANOVA, Kruskal-Wallis's test and multiple t-test). This is particularly evident when approaching the end stage, where a significantly higher score obtained at age P475 until P525 was observed for the combo-treated mice compared to the placebo group. Indeed, at this late symptomatic stage, most of the placebo mice were no longer able to run onto the rotating wheel while more than half of the darifenacin-treated group were still able to run. Interestingly, there was a tendency of improved motor behavior at the Rotarod for the ARGX-119+DMSO treated mice from the ages of P510 to P525 (p=0.07, p=0.06 and p=0.053 respectively). These results demonstrated the beneficial impact of the combo-treatment compared to other mono-treatments and the placebo group.

Second, the grip strength measured to assess if the combined treatment ameliorated the general strength of the animals (FIG. 1C). Mice of all groups began the trial with a similar grip strength force. However, combo-treated mice performed better than the mice in the placebo group as shown by the larger grip strength generated at P460 until the end of the preclinical trial (FIG. D; ARGX-119+DMSO N=5, ARGX-119+darifenacin N=4, PBS+darifenacin N=5, Placebo N=5, One-way ANOVA, p<0.05, Tukey's test and multiple t-test). Interestingly, at P507, PBS+darifenacin treated mice had a significant increase of grip strength compared to the placebo group.

Third, it was tested whether the combined-treatment of ARGX-119 antibody with darifenacin had an impact on the general condition of the mice. To this end, the changes in the body weight of the animals were monitored, a metric that is directly related to the progression of the disease and survival whereby animals present an important gradual body weight loss after symptoms onset. However, no difference was observed between the groups (ARGX-119+DMSO N=5, ARGX-119+darifenacin N=4, PBS+darifenacin N=5, Placebo N=5, p>0.05, One-way ANOVA, Tukey's test and multiple t-test).

### Example 2.1: Combo-Treatment Improves Neuromuscular Contractile Muscle Force and NMJ Efficacy

Improved contractile muscle properties are strong indicators that the muscle and NMJ functions should also be improved by the combined treatment. Two muscles with different properties and resistance to the disease were investigated. The EDL was used as a fast-twitch fatigable muscle that is vulnerable to the disease and the SOL as a slow-twitch fatigue resistant muscle that is also more resistant to the disease. A muscle force transducer was used to measure the force generated by the muscles upon stimulation of the motor nerve and/or direct muscle stimulation (see FIG. 2A). With this system, stimulation of the motor nerve at various frequencies elicits muscle contraction through NMJ efficacy, reflecting the strength of contractile fibers associated with innervated NMJs only. Muscle stimulation, on the contrary, depolarizes all muscle fibers and reflect maximal twitch force of all the muscle, independent of the innervation status. This method is especially useful to characterize diseases like ALS presenting NMJ and muscular deficits (35, 36).

### Fast-Fatigable EDL Muscle:

First, a standard protocol of stimulation was performed to generate a force frequency curve (5 Hz-300 Hz) to characterize NMJ efficacy following the ARGX-119 and darifenacin chronic treatments. Muscle force generated by the contractions elicited by the stimulation of the motor nerve and NMJ activation was significantly higher than EDL from the placebo group (p<0.001) or the ARGX-119+DMSO group (p<0.05) during the protocol (FIG. 2B; ARGX-119+DMSO N=5, ARGX-119+darifenacin N=4, Placebo N=5; Repeated One-way ANOVA, Bonferroni post hoc test). Indeed, the generated twitch force were 66.3±15.7 mN for the combo-treatment, 47.2±12.4 mN for the placebo group and 53.6±14.9 mN for the ARGX-119+DMSO group. There was no significant difference with the group ARGX-119 antibody group compared to placebo treated mice.

For the direct muscle stimulation, significant differences between the combo-treated mice and the other groups were observed at higher frequencies (FIG. 2C). The combo-treated group showed significant high peak force (mN) compared to the ARGX-119 and DMSO group (p<0.05) as well as the placebo group (p<0.001). This is indicative of the preservation of the fast-twitch properties of the EDL (37, 38) (FIG. 2C, ARGX-119+DMSO N=5, ARGX-119+darifenacin N=4, Placebo N=5, Repeated One-way ANOVA, Bonferroni post hoc test and multiple t-test). Interestingly, this increase in contractile force was also observed with a better preservation of the EDL muscle weight in the combo-treated animals compared to the placebo group as shown in FIG. 2E.

Next, the proportion of the muscle capacity that is used by the neuromuscular system upon nerve stimulation was determined. This was expressed as the contractile capacity ratio. This ratio is at 100% in WT mice, indicating that the neuronal control of the muscle recruits 100% of its contractile capacity. Hence, if treatments improve NMJ innervation, resulting in an increase in force generated, it is posited that this ratio should be higher in EDL muscles from combined-treated animals compared to the other groups. As shown in FIG. 2D, the ratio was significantly higher for the EDL of ARGX-119+DMSO mice, with 61.1±1.0% compared to 52.3±1.8% (p<0.001) for combined-treated mice and to 53.3±3.6% (p<0.0001) for the placebo group (ARGX-119+DMSO N=5, ARGX-119+darifenacin N=4, Placebo N=5, One-way ANOVA, Kruskal-Wallis's test).

### Slow Twitch SOL Muscle:

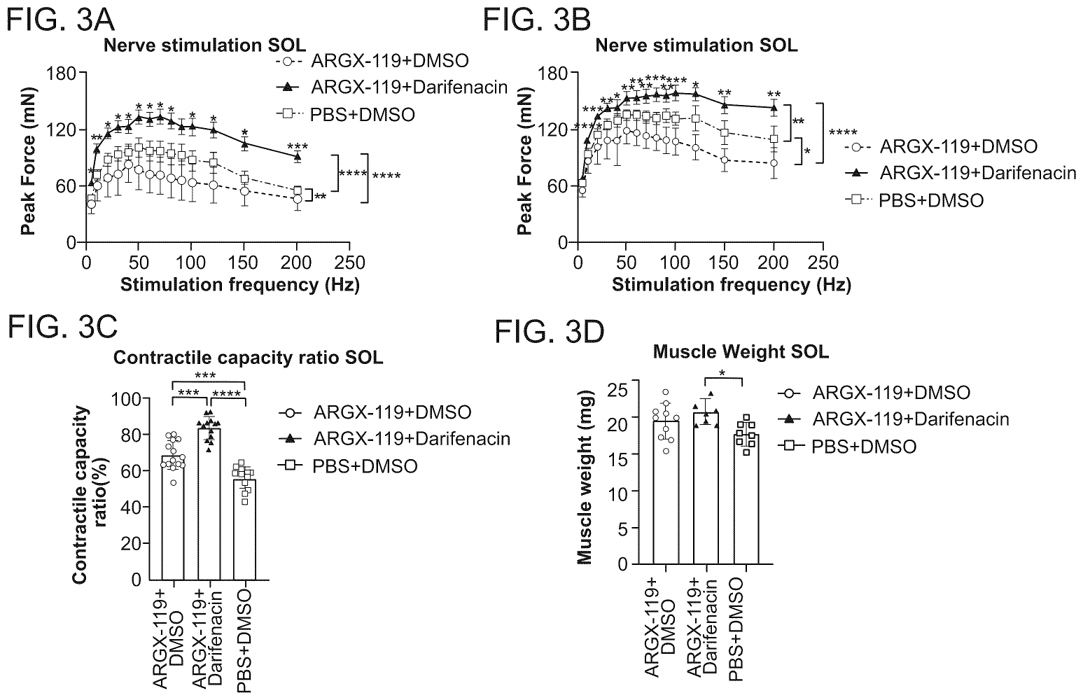

Then, the same protocol was performed, but for the SOL muscle (FIG. 3). Combined-treated group demonstrated significantly higher twitch forces than from the ARGX-119+DMSO treated group (p<0.0001) and placebo group (p<0.0001) during the protocol for the nerve stimulation (FIG. 3A; ARGX-119+DMSO N=5, ARGX-119+darifenacin N=4, Placebo N=5, Repeated One-way ANOVA and multiple t-test). However, the ARGX-119+DMSO treated group generated smaller contraction force forces in comparison to placebo (p<0.05). The generated twitch force was 114.5±3.5 mN for the combo-treatment, 83.1±4.5 mN for the placebo group and 64.6±5.1 mN for the ARGX-119+DMSO group. In the case of the direct muscle stimulation, combined-treated group demonstrated once again significantly higher twitch forces (140.6±2.1 mN) than from the ARGX-119+DMSO treated group (98.3±2.1 mN; p<0.01) and placebo group (119.5±1.6 mN; p<0.0001) during the protocol for the nerve stimulation (FIG. 3B; ARGX-119+DMSO N=5, ARGX-119+darifenacin N=4, Placebo N=5, Repeated One-way ANOVA). There was also a significant difference between the ARGX-119+DMSO treated group and placebo group (p<0.05).

The contractile capacity ratio (FIG. 3C) was significantly higher in combined-treated mice, with 82.0±2.8% compared to 68.4±7.8% for the ARGX-119-treated group (p<0.0001) and 56.1± for the control mice (p<0.0001; ARGX-119+DMSO N=5, ARGX-119+darifenacin N=4, Placebo N=5, Repeated One-way ANOVA). Interestingly, this increase in contractile force and contractile capacity ratio was observed with a better preservation of the SOL muscle weight in treated animals. Indeed, the ARGX-119+darifenacin treated mice had better preserved muscle weight compared to the Placebo group (p<0.05; ARGX-119+DMSO N=5, ARGX-119+darifenacin N=4, Placebo N=5, One-way ANOVA). Interestingly, there was also a significant difference between the ARGX-119+DMSO treated group and placebo group (p<0.001), where the SOL from the ARGX-119 generated the better contractile capacity ratio than the placebo ones.

Overall, these results suggest that the combo treatment improves muscle and neuromuscular contractile forces as well as muscle weight for both muscles, but the contractile capacity ratio was increased for EDL and SOL only for the ARGX-119+DMSO group.

### Example 2.2: ARGX-119 Antibody Combined with Darifenacin Preserves Muscle Fatigue Properties

In addition to the force generated, a muscle is also characterised by its resistance to fatigue. For instance, fast-twitch muscles composed mainly of fast fatigable motor units like the EDL show higher fatigue in comparison to slow twitch muscle like the SOL (37). In ALS, alteration at the type of innervation (from fast to slow twitch) and in the properties of muscles themselves alter the fatigue properties, rendering them more resistant. Since the treatment by the combined ARGX-119+darifenacin significantly preserved muscle strength, next investigated the resistance to fatigue of the EDL and the SOL muscles.

A fatigue stimulation protocol was used, followed by a 30 min recovery period (see FIG. 4A). EDL muscles from the placebo-treated group showed an atypical resistance to fatigue when the nerve is directly stimulated. However, as revealed by a delayed recovery, EDL muscles from the combined ARGX-119+darifenacin-treated mice showed a level of fatigue that is more typical for this type of fast twitch muscle (FIG. 4B; ARGX-119+darifenacin N=4, ARGX-119+DMSO N=5, Placebo N=5; p<0.05, one-way ANOVA, Kruskal-Wallis's test and multiple t-test). Interestingly, during the recovery, there was a significant difference between the ARGX-119 treatment and the placebo group (p<0.05), where the ARGX-119+DMSO treated muscle showed a more typical fatigue. This suggests that the used of a combined treatment as well as a mono-treatment like ARGX-119 antibody improved muscle properties. However, no difference was found in the rate of fatigue and recovery of nerve+muscle stimulation, when all muscle fibers are recruited (FIG. 4C; ARGX-119+darifenacin N=4, ARGX-119+DMSO N=5, Placebo N=5; p>0.05; one-way ANOVA, Kruskal-Wallis's test).

SOL is a slow-twitch and resistant muscle that is expected to be also more resistant from the denervation seen in degenerative diseases compared to the fast-twitch muscle EDL and to recover faster. For the nerve stimulation of the SOL muscle, the placebo group showed a more pronounced fatigue as revealed by a slower fatigue recovery in comparison to the ARGX-119+darifenacin treated-group, which is atypical for this fatigue-resistant muscle. Hence, the combo treatment restored the fatigue-resistant properties of the SOL muscle. (FIGS. 4D-4E; ARGX-119+darifenacin N=4, ARGX-119+DMSO N=5, Placebo N=5; p<0.01; one-way ANOVA, Kruskal-Wallis's test and multiple t-test). For the nerve+muscle stimulation, there is a significant difference between the ARGX-119+darifenacin and the ARGX-119+DMSO groups (p<0.001) and the ARGX-119+DMSO and the placebo groups (p<0.0001).

1-45. (canceled)

46. A method of treating a neuromuscular disorder in a human subject, the method comprises administering an anti-MuSK antibody or an antigen-binding fragment thereof.

47. The method of claim 46, wherein the antibody or antigen-binding fragment thereof binds the MuSK Frizzled (Fz)-like domain sequence of the amino acid sequence of SEQ ID NO:
129.

48. The method of claim 46, wherein the antibody or antigen-binding fragment thereof comprises a human IgG constant Fc region at least 80% identical to the amino acid sequences of SEQ ID NO: 266 or 267.

49. The method of claim 46, wherein the antibody or antigen-binding fragment thereof is an agonist MuSK antibody and/or has reduced or eliminated effector function.

50. The method of claim 48, wherein the amino acid sequence of the human IgG constant Fc region comprises one or more of the following mutations (all numbered according to the EU numbering system): an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution.

51. The method of claim 50, wherein the antibody or antigen-binding fragment thereof comprises an L234A and L235A mutation numbered according to the EU numbering system.

52. The method of claim 46, wherein the antibody or antigen-binding fragment thereof comprises:
a) a heavy chain variable domain (VH) comprising an amino acid sequence that is at least 80% identical to the amino acid sequence of SEQ ID NO: 234 and
b) a light chain variable domain (VL) comprising an amino acid sequence that is at least 80% identical to the amino acid sequence of SEQ ID NO: 235.

53. The method of claim 47,
(a) wherein the VH comprises:
(i) a CDR-H1 comprising the amino acid sequence of SEQ ID NO:147 or has 1, 2, 3, 4 or 5 amino acid substitutions relative to the amino acid sequence of SEQ ID NO: 147,
(ii) a CDR-H2 comprising the amino acid sequence of SEQ ID NO: 153 or has 1, 2, 3, 4 or amino acid substitutions relative to the amino acid sequence of SEQ ID NO: 153, and
(iii) a CDR-H3 comprising the amino acid sequence of SEQ ID NO: 156 or has 1, 2, 3, 4 or amino acid substitutions relative to the amino acid sequence of SEQ ID NO:156; and
(b) wherein the VL comprises:
(i) a CDR-L1 comprising the amino acid sequence of SEQ ID NO: 159 or has 1, 2, 3, 4 or 5 amino acid substitutions relative to the amino acid sequence of SEQ ID NO: 159,
(ii) a CDR-L2 comprising the amino acid sequence of SEQ ID NO: 172 or has 1, 2, 3, 4 or 5 amino acid substitutions relative to the amino acid sequence of SEQ ID NO: 172, and
(iii) a CDR-L3 comprising the amino acid sequence of SEQ ID NO: 195 or has 1, 2, 3, 4 or amino acid substitutions relative to the amino acid sequence of SEQ ID NO:195.

54. The method of claim 53,
(a) wherein the VH comprises:
(i) the CDR-H1 comprising the amino acid sequence of SEQ ID NO: 147,
(ii) the CDR-H2 comprising the amino acid sequence of SEQ ID NO: 153, and
(iii) the CDR-H3 comprising the amino acid sequence of SEQ ID NO: 156; and
(b) wherein the VL comprises:
(i) the CDR-L1 comprising the amino acid sequence of SEQ ID NO: 159,
(ii) the CDR-L2 comprising the amino acid sequence of SEQ ID NO: 172, and
(iii) the CDR-L3 comprising the amino acid sequence of SEQ ID NO: 195.

55. The method of claim 52, wherein the VH comprises SEQ ID NO: 234, and the VL comprises SEQ ID NO: 235.

56. The method of claim 46, wherein the antibody or fragment thereof comprises a VH and a VL as identified in table 3 and/or a CDR as identified in table 1 or 2.

57. A method of treating a neuromuscular disorder in a human subject, the method comprising administering an anti-MuSK antibody or fragment thereof, wherein the antibody or fragment thereof comprises:
(i) a full length heavy chain comprising an amino acid sequence that is at least 80% identical or similar to the amino acid sequence of SEQ ID NO: 270 and a full length light chain comprising an amino acid sequence that is at least 80% identical or similar to the amino acid sequence of SEQ ID NO: 271, or
(ii) a full length heavy chain comprising an amino acid sequence that is at least 80% identical or similar to the amino acid sequence of SEQ ID NO: 268 and a full length light chain comprising an amino acid sequence that is at least 80% identical or similar to the amino acid sequence of SEQ ID NO: 269;
(iii) wherein the full length heavy chain comprises one or more of the following mutations (all numbered according to the EU numbering system): an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

58. The method of claim 57, wherein the antibody or antigen-binding fragment thereof comprises a full length heavy chain comprising the amino acid sequence of SEQ ID NO: 270 and a full length light chain comprising the amino acid sequence of SEQ ID NO: 271, and wherein the full length heavy chain comprises L234A and L235A mutations numbered according the EU numbering system.

59. The method according to claim 57, wherein the antibody or antigen-binding fragment thereof comprises a full length heavy chain comprising the amino acid sequence of SEQ ID NO: 268 and a full length light chain comprising the amino acid sequence of SEQ ID NO: 269, wherein the full length heavy chain comprises L234A and L235A mutations numbered according the EU numbering system.


SEQUENCE LISTING (279 sequences)

SEQ ID NO: 1 - protein, 5 aa, synthetic construct
  REGION 1..5: note = X1 HCDR1
SSSIH

SEQ ID NO: 2 - protein, 5 aa, synthetic construct
  REGION 1..5: note = X2 HCDR1
SSSIH

SEQ ID NO: 3 - protein, 5 aa, synthetic construct
  REGION 1..5: note = X3 HCDR1
SSSIH

SEQ ID NO: 4 - protein, 5 aa, synthetic construct
  REGION 1..5: note = X4 HCDR1
SSSIH

SEQ ID NO: 5 - protein, 5 aa, synthetic construct
  REGION 1..5: note = X5 HCDR1
SSSIH

SEQ ID NO: 6 - protein, 5 aa, synthetic construct
  REGION 1..5: note = X6 HCDR1
SSSIH

SEQ ID NO: 7 - protein, 5 aa, synthetic construct
  REGION 1..5: note = X7 HCDR1
SSSIH

SEQ ID NO: 8 - protein, 5 aa, synthetic construct
  REGION 1..5: note = X8 HCDR1
SSSIH

SEQ ID NO: 9 - protein, 5 aa, synthetic construct
  REGION 1..5: note = X9 HCDR1
SSSIH

SEQ ID NO: 10 - protein, 5 aa, synthetic construct
  REGION 1..5: note = X10 HCDR1
SSSIH

SEQ ID NO: 11 - protein, 5 aa, synthetic construct
  REGION 1..5: note = X11 HCDR1
SSSIH

SEQ ID NO: 12 - protein, 5 aa, synthetic construct
  REGION 1..5: note = X12 HCDR1
SSSIH

SEQ ID NO: 13 - protein, 5 aa, synthetic construct
  REGION 1..5: note = X13 HCDR1
SSSIH

SEQ ID NO: 14 - protein, 5 aa, synthetic construct
  REGION 1..5: note = X14 HCDR1
SSSIH

SEQ ID NO: 15 - protein, 5 aa, synthetic construct
  REGION 1..5: note = X15 HCDR1
SSSIH

SEQ ID NO: 16 - protein, 5 aa, synthetic construct
  REGION 1..5: note = X16 HCDR1
SSSIH

SEQ ID NO: 17 - protein, 17 aa, synthetic construct
  REGION 1..17: note = X1 HCDR2
SISSSSGSTSYADSVKG

SEQ ID NO: 18 - protein, 17 aa, synthetic construct
  REGION 1..17: note = X2 HCDR2
SISSSYGSTSYADSVKG

SEQ ID NO: 19 - protein, 17 aa, synthetic construct
  REGION 1..17: note = X3 HCDR2
SISSSSGYTYYADSVKG

SEQ ID NO: 20 - protein, 17 aa, synthetic construct
  REGION 1..17: note = X4 HCDR2
SISSSSGSTYYADSVKG

SEQ ID NO: 21 - protein, 17 aa, synthetic construct
  REGION 1..17: note = X5 HCDR2
SISSSSGSTSYADSVKG

SEQ ID NO: 22 - protein, 17 aa, synthetic construct
  REGION 1..17: note = X6 HCDR2
SISSYSGYTYYADSVKG

SEQ ID NO: 23 - protein, 17 aa, synthetic construct
  REGION 1..17: note = X7 HCDR2
SISSSYSSTYYADSVKG

SEQ ID NO: 24 - protein, 17 aa, synthetic construct
  REGION 1..17: note = X8 HCDR2
SISSSSGSTSYADSVKG

SEQ ID NO: 25 - protein, 17 aa, synthetic construct
  REGION 1..17: note = X9 HCDR2
SIYSSSGSTYYADSVKG

SEQ ID NO: 26 - protein, 17 aa, synthetic construct
  REGION 1..17: note = X10 HCDR2
SISSSYSSTSYADSVKG

SEQ ID NO: 27 - protein, 17 aa, synthetic construct
  REGION 1..17: note = X11 HCDR2
SISSYSGSTYYADSVKG

SEQ ID NO: 28 - protein, 17 aa, synthetic construct
  REGION 1..17: note = X12 HCDR2
YISPSSGYTSYADSVKG

SEQ ID NO: 29 - protein, 17 aa, synthetic construct
  REGION 1..17: note = X13 HCDR2
SISSSSGSTSYADSVKG

SEQ ID NO: 30 - protein, 17 aa, synthetic construct
  REGION 1..17: note = X14 HCDR2
SISSSSGYTYYADSVKG

SEQ ID NO: 31 - protein, 17 aa, synthetic construct
  REGION 1..17: note = X15 HCDR2
SISSSYGSTSYADSVKG

SEQ ID NO: 32 - protein, 17 aa, synthetic construct
  REGION 1..17: note = X16 HCDR2
SIYSSYGYTSYADSVKG

SEQ ID NO: 33 - protein, 17 aa, synthetic construct
  REGION 1..17: note = X1 HCDR3
KYWSQYYWAHYYGGLDY

SEQ ID NO: 34 - protein, 15 aa, synthetic construct
  REGION 1..15: note = X2 HCDR3
SEGDRYVSGYMGMDY

SEQ ID NO: 35 - protein, 15 aa, synthetic construct
  REGION 1..15: note = X3 HCDR3
SWYEMWMSGYFGFDY

SEQ ID NO: 36 - protein, 15 aa, synthetic construct
  REGION 1..15: note = X4 HCDR3
GEHDYYVFGYLGMDY

SEQ ID NO: 37 - protein, 20 aa, synthetic construct
  REGION 1..20: note = X5 HCDR3
SYTMFYYGGWYGSGYFGMDY

SEQ ID NO: 38 - protein, 15 aa, synthetic construct
  REGION 1..15: note = X6 HCDR3
TYGSYYVSSYTGMDY

SEQ ID NO: 39 - protein, 15 aa, synthetic construct
  REGION 1..15: note = X7 HCDR3
LAGLYHYPGYLGLDY

SEQ ID NO: 40 - protein, 19 aa, synthetic construct
  REGION 1..19: note = X8 HCDR3
SWSYHPWYYHVGWYTGLDY

SEQ ID NO: 41 - protein, 15 aa, synthetic construct
  REGION 1..15: note = X9 HCDR3
SGGEFYITSYYGMDY

SEQ ID NO: 42 - protein, 15 aa, synthetic construct
  REGION 1..15: note = X10 HCDR3
KYYRWRHNKYQGFDY

SEQ ID NO: 43 - protein, 15 aa, synthetic construct
  REGION 1..15: note = X11 HCDR3
SWGSYYVSGFVGFDY

SEQ ID NO: 44 - protein, 17 aa, synthetic construct
  REGION 1..17: note = X12 HCDR3
QYWVPQWWITQYFGMDY

SEQ ID NO: 45 - protein, 15 aa, synthetic construct
  REGION 1..15: note = X13 HCDR3
SSEHWYTIGYYGIDY

SEQ ID NO: 46 - protein, 15 aa, synthetic construct
  REGION 1..15: note = X14 HCDR3
GSHHWFLWIYSGLDY

SEQ ID NO: 47 - protein, 15 aa, synthetic construct
  REGION 1..15: note = X15 HCDR3
SEGDRYVSGYMGMDY

SEQ ID NO: 48 - protein, 16 aa, synthetic construct
  REGION 1..16: note = X16 HCDR3
NWGYYMYWGWYYALDY

SEQ ID NO: 49 - protein, 11 aa, synthetic construct
  REGION 1..11: note = X1 LCDR1
RASQSVSSAVA

SEQ ID NO: 50 - protein, 11 aa, synthetic construct
  REGION 1..11: note = X2 LCDR1
RASQSVSSAVA

SEQ ID NO: 51 - protein, 11 aa, synthetic construct
  REGION 1..11: note = X3 LCDR1
RASQSVSSAVA

SEQ ID NO: 52 - protein, 11 aa, synthetic construct
  REGION 1..11: note = X4 LCDR1
RASQSVSSAVA

SEQ ID NO: 53 - protein, 11 aa, synthetic construct
  REGION 1..11: note = X5 LCDR1
RASQSVSSAVA

SEQ ID NO: 54 - protein, 11 aa, synthetic construct
  REGION 1..11: note = X6 LCDR1
RASQSVSSAVA

SEQ ID NO: 55 - protein, 11 aa, synthetic construct
  REGION 1..11: note = X7 LCDR1
RASQSVSSAVA

SEQ ID NO: 56 - protein, 11 aa, synthetic construct
  REGION 1..11: note = X8 LCDR1
RASQSVSSAVA

SEQ ID NO: 57 - protein, 11 aa, synthetic construct
  REGION 1..11: note = X9 LCDR1
RASQSVSSAVA

SEQ ID NO: 58 - protein, 11 aa, synthetic construct
  REGION 1..11: note = X10 LCDR1
RASQSVSSAVA

SEQ ID NO: 59 - protein, 11 aa, synthetic construct
  REGION 1..11: note = X11 LCDR1
RASQSVSSAVA

SEQ ID NO: 60 - protein, 11 aa, synthetic construct
  REGION 1..11: note = X12 LCDR1
RASQSVSSAVA

SEQ ID NO: 61 - protein, 11 aa, synthetic construct
  REGION 1..11: note = X13 LCDR1
RASQSVSSAVA

SEQ ID NO: 62 - protein, 11 aa, synthetic construct
  REGION 1..11: note = X14 LCDR1
RASQSVSSAVA

SEQ ID NO: 63 - protein, 11 aa, synthetic construct
  REGION 1..11: note = X15 LCDR1
RASQSVSSAVA

SEQ ID NO: 64 - protein, 11 aa, synthetic construct
  REGION 1..11: note = X16 LCDR1
RASQSVSSAVA

SEQ ID NO: 65 - protein, 7 aa, synthetic construct
  REGION 1..7: note = X1 LCDR2
SASSLYS

SEQ ID NO: 66 - protein, 7 aa, synthetic construct
  REGION 1..7: note = X2 LCDR2
SASSLYS

SEQ ID NO: 67 - protein, 7 aa, synthetic construct
  REGION 1..7: note = X3 LCDR2
SASSLYS

SEQ ID NO: 68 - protein, 7 aa, synthetic construct
  REGION 1..7: note = X4 LCDR2
SASSLYS

SEQ ID NO: 69 - protein, 7 aa, synthetic construct
  REGION 1..7: note = X5 LCDR2
SASSLYS

SEQ ID NO: 70 - protein, 7 aa, synthetic construct
  REGION 1..7: note = X6 LCDR2
SASSLYS

SEQ ID NO: 71 - protein, 7 aa, synthetic construct
  REGION 1..7: note = X7 LCDR2
SASSLYS

SEQ ID NO: 72 - protein, 7 aa, synthetic construct
  REGION 1..7: note = X8 LCDR2
SASSLYS

SEQ ID NO: 73 - protein, 7 aa, synthetic construct
  REGION 1..7: note = X9 LCDR2
SASSLYS

SEQ ID NO: 74 - protein, 7 aa, synthetic construct
  REGION 1..7: note = X10 LCDR2
SASSLYS

SEQ ID NO: 75 - protein, 7 aa, synthetic construct
  REGION 1..7: note = X11 LCDR2
SASSLYS

SEQ ID NO: 76 - protein, 7 aa, synthetic construct
  REGION 1..7: note = X12 LCDR2
SASSLYS

SEQ ID NO: 77 - protein, 7 aa, synthetic construct
  REGION 1..7: note = X13 LCDR2
SASSLYS

SEQ ID NO: 78 - protein, 7 aa, synthetic construct
  REGION 1..7: note = X14 LCDR2
SASSLYS

SEQ ID NO: 79 - protein, 7 aa, synthetic construct
  REGION 1..7: note = X15 LCDR2
SASSLYS

SEQ ID NO: 80 - protein, 7 aa, synthetic construct
  REGION 1..7: note = X16 LCDR2
SASSLYS

SEQ ID NO: 81 - protein, 9 aa, synthetic construct
  REGION 1..9: note = X1 LCDR3
QQSSSSLIT

SEQ ID NO: 82 - protein, 9 aa, synthetic construct
  REGION 1..9: note = X2 LCDR3
QQSGVWLIT

SEQ ID NO: 83 - protein, 9 aa, synthetic construct
  REGION 1..9: note = X3 LCDR3
QQSSSSLIT

SEQ ID NO: 84 - protein, 11 aa, synthetic construct
  REGION 1..11: note = X4 LCDR3
QQSYKPGALIT

SEQ ID NO: 85 - protein, 9 aa, synthetic construct
  REGION 1..9: note = X5 LCDR3
QQSSSSLIT

SEQ ID NO: 86 - protein, 9 aa, synthetic construct
  REGION 1..9: note = X6 LCDR3
QQSSSSLIT

SEQ ID NO: 87 - protein, 10 aa, synthetic construct
  REGION 1..10: note = X7 LCDR3
QQSSRSSLLT

SEQ ID NO: 88 - protein, 9 aa, synthetic construct
  REGION 1..9: note = X8 LCDR3
QQSSSSLIT

SEQ ID NO: 89 - protein, 9 aa, synthetic construct
  REGION 1..9: note = X9 LCDR3
QQSSSSLIT

SEQ ID NO: 90 - protein, 9 aa, synthetic construct
  REGION 1..9: note = X10 LCDR3
QQSLWYPVT

SEQ ID NO: 91 - protein, 9 aa, synthetic construct
  REGION 1..9: note = X11 LCDR3
QQNSYYLIT

SEQ ID NO: 92 - protein, 9 aa, synthetic construct
  REGION 1..9: note = X12 LCDR3
QQSSSSLIT

SEQ ID NO: 93 - protein, 11 aa, synthetic construct
  REGION 1..11: note = X13 LCDR3
QQSYGSFSLIT

SEQ ID NO: 94 - protein, 9 aa, synthetic construct
  REGION 1..9: note = X14 LCDR3
QQGSYHLIT

SEQ ID NO: 95 - protein, 9 aa, synthetic construct
  REGION 1..9: note = X15 LCDR3
QQSGVWLIT

SEQ ID NO: 96 - protein, 11 aa, synthetic construct
  REGION 1..11: note = X16 LCDR3
QQWSSAQALIT

SEQ ID NO: 97 - protein, 126 aa, synthetic construct
  REGION 1..126: note = X1 VH
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARKYWSQYYWAHYYGGLDYWGQGTLVTVSS

SEQ ID NO: 98 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIK

SEQ ID NO: 99 - protein, 124 aa, synthetic construct
  REGION 1..124: note = X2 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYMGMDYWGQGTLVTVSS

SEQ ID NO: 100 - protein, 107 aa, synthetic construct
  REGION 1..107: note = X2 VL
DIQMTQSPSSLSASVGDRVTITCRASQSVSSAVAWYQQKPGKAPKLLIYSASSLYSGVPSRFSGSRSGTDFTLTISSLQPEDFATYYCQQSGVWLITFGQGTKVEIK

SEQ ID NO: 101 - protein, 124 aa, synthetic construct
  REGION 1..124: note = X3 VH
EVQLVESGGGLVQPGGSLRLSCAASGFTISSSSIHWVRQAPGKGLEWVASISSSSGYTYYADSVKGRFTISADTSKNTAYLQMNSLRAEDTAVYYCARSWYEMWMSGYFGFDYWGQGTLVTVSS

SEQ ID NO: 102 - protein, 107 aa, synthetic construct; identical to SEQ ID NO: 98
  REGION 1..107: note = VL

SEQ ID NO: 103 - protein, 124 aa, synthetic construct
  REGION 1..124: note = X4 VH
EVQLVESGGGLVQPGGSLRLSCAASGFTVSSSSIHWVRQAPGKGLEWVASISSSSGSTYYADSVKGRFTISADTSKNTAYLQMNSLRAEDTAVYYCARGEHDYYVFGYLGMDYWGQGTLVTVSS

SEQ ID NO: 104 - protein, 109 aa, synthetic construct
  REGION 1..109: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSYKPGALITFGQGTKVEIK

SEQ ID NO: 105 - protein, 129 aa, synthetic construct
  REGION 1..129: note = X5 VH
EVQLVESGGGLVQPGGSLRLSCAASGFTFYSSSIHWVRQAPGKGLEWVASISSSSGSTSYADSVKGRFTISADTSKNTAYLQMNSLRAEDTAVYYCARSYTMFYYGGWYGSGYFGMDYWGQGTLVTVSS

SEQ ID NO: 106 - protein, 107 aa, synthetic construct; identical to SEQ ID NO: 98
  REGION 1..107: note = VL

SEQ ID NO: 107 - protein, 124 aa, synthetic construct
  REGION 1..124: note = X6 VH
EVQLVESGGGLVQPGGSLRLSCAASGFTFSSSSIHWVRQAPGKGLEWVASISSYSGYTYYADSVKGRFTISADTSKNTAYLQMNSLRAEDTAVYYCARTYGSYYVSSYTGMDYWGQGTLVTVSS

SEQ ID NO: 108 - protein, 107 aa, synthetic construct; identical to SEQ ID NO: 98
  REGION 1..107: note = VL

SEQ ID NO: 109 - protein, 124 aa, synthetic construct
  REGION 1..124: note = X7 VH
EVQLVESGGGLVQPGGSLRLSCAASGFTLYSSSIHWVRQAPGKGLEWVASISSSYSSTYYADSVKGRFTISADTSKNTAYLQMNSLRAEDTAVYYCARLAGLYHYPGYLGLDYWGQGTLVTVSS

SEQ ID NO: 110 - protein, 108 aa, synthetic construct
  REGION 1..108: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSSRSSLLTFGQGTKVEIK

SEQ ID NO: 111 - protein, 128 aa, synthetic construct
  REGION 1..128: note = X8 VH
EVQLVESGGGLVQPGGSLRLSCAASGFTVSSSSIHWVRQAPGKGLEWVASISSSSGSTSYADSVKGRFTISADTSKNTAYLQMNSLRAEDTAVYYCARSWSYHPWYYHVGWYTGLDYWGQGTLVTVSS

SEQ ID NO: 112 - protein, 107 aa, synthetic construct; identical to SEQ ID NO: 98
  REGION 1..107: note = VL

SEQ ID NO: 113 - protein, 124 aa, synthetic construct
  REGION 1..124: note = X9 VH
EVQLVESGGGLVQPGGSLRLSCAASGFTFSSSSIHWVRQAPGKGLEWVASIYSSSGSTYYADSVKGRFTISADTSKNTAYLQMNSLRAEDTAVYYCARSGGEFYITSYYGMDYWGQGTLVTVSS

SEQ ID NO: 114 - protein, 107 aa, synthetic construct; identical to SEQ ID NO: 98
  REGION 1..107: note = VL

SEQ ID NO: 115 - protein, 124 aa, synthetic construct
  REGION 1..124: note = X10 VH
EVQLVESGGGLVQPGGSLRLSCAASGFTFSSSSIHWVRQAPGKGLEWVASISSSYSSTSYADSVKGRFTISADTSKNTAYLQMNSLRAEDTAVYYCARKYYRWRHNKYQGFDYWGQGTLVTVSS

SEQ ID NO: 116 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSLWYPVTFGQGTKVEIK

SEQ ID NO: 117 - protein, 124 aa, synthetic construct
  REGION 1..124: note = X11 VH
EVQLVESGGGLVQPGGSLRLSCAASGFTISSSSIHWVRQAPGKGLEWVASISSYSGSTYYADSVKGRFTISADTSKNTAYLQMNSLRAEDTAVYYCARSWGSYYVSGFVGFDYWGQGTLVTVSS

SEQ ID NO: 118 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQNSYYLITFGQGTKVEIK

SEQ ID NO: 119 - protein, 126 aa, synthetic construct
  REGION 1..126: note = X12 VH
EVQLVESGGGLVQPGGSLRLSCAASGFTISSSSIHWVRQAPGKGLEWVAYISPSSGYTSYADSVKGRFTISADTSKNTAYLQMNSLRAEDTAVYYCARQYWVPQWWITQYFGMDYWGQGTLVTVSS

SEQ ID NO: 120 - protein, 107 aa, synthetic construct; identical to SEQ ID NO: 98
  REGION 1..107: note = VL

SEQ ID NO: 121 - protein, 124 aa, synthetic construct
  REGION 1..124: note = X13 VH
EVQLVESGGGLVQPGGSLRLSCAASGFTISSSSIHWVRQAPGKGLEWVASISSSSGSTSYADSVKGRFTISADTSKNTAYLQMNSLRAEDTAVYYCARSSEHWYTIGYYGIDYWGQGTLVTVSS

SEQ ID NO: 122 - protein, 109 aa, synthetic construct
  REGION 1..109: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSYGSFSLITFGQGTKVEIK

SEQ ID NO: 123 - protein, 124 aa, synthetic construct
  REGION 1..124: note = X14 VH
EVQLVESGGGLVQPGGSLRLSCAASGFTFSSSSIHWVRQAPGKGLEWVASISSSSGYTYYADSVKGRFTISADTSKNTAYLQMNSLRAEDTAVYYCARGSHHWFLWIYSGLDYWGQGTLVTVSS

SEQ ID NO: 124 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQGSYHLITFGQGTKVEIK

SEQ ID NO: 125 - protein, 124 aa, synthetic construct; identical to SEQ ID NO: 99
  REGION 1..124: note = X15 VH

SEQ ID NO: 126 - protein, 107 aa, synthetic construct; identical to SEQ ID NO: 100
  REGION 1..107: note = VL

SEQ ID NO: 127 - protein, 125 aa, synthetic construct
  REGION 1..125: note = X16 VH
EVQLVESGGGLVQPGGSLRLSCAASGFTFSSSSIHWVRQAPGKGLEWVASIYSSYGYTSYADSVKGRFTISADTSKNTAYLQMNSLRAEDTAVYYCARNWGYYMYWGWYYALDYWGQGTLVTVSS

SEQ ID NO: 128 - protein, 109 aa, synthetic construct
  REGION 1..109: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQWSSAQALITFGQGTKVEIK

SEQ ID NO: 129 - protein, 869 aa, Homo sapiens
MRELVNIPLVHILTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMTVIISIMSSFAIFVLLTITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 130 - protein, 139 aa, synthetic construct
  REGION 1..139: note = Fz-like domain of MuSK
DNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARL

SEQ ID NO: 131 - protein, 124 aa, synthetic construct
  REGION 1..124: note = X17 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVFGYLGMDYWGQGTLVTVSS

SEQ ID NO: 132 - protein, 109 aa, synthetic construct; identical to SEQ ID NO: 104
  REGION 1..109: note = X17 VL

SEQ ID NO: 133 - protein, 126 aa, synthetic construct
  REGION 1..126: note = X18 VH
EVQLVESGGGLVQPGGSLRLSCAASGFTISSSSIHWVRQAPGKGLEWVASISSSSGYTSYADSVKGRFTISADTSKNTAYLQMNSLRAEDTAVYYCARKYSKRAYPDYYWRGLDYWGQGTLVTVSS

SEQ ID NO: 134 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSYWWPITFGQGTKVEIK

SEQ ID NO: 135 - protein, 5 aa, synthetic construct
  REGION 1..5: note = X17 HCDR1
YSSIH

SEQ ID NO: 136 - protein, 5 aa, synthetic construct
  REGION 1..5: note = X18 HCDR1
SSSIH

SEQ ID NO: 137 - protein, 17 aa, synthetic construct
  REGION 1..17: note = X17 HCDR2
SIYSSSGSTYYADSVKG

SEQ ID NO: 138 - protein, 17 aa, synthetic construct
  REGION 1..17: note = X18 HCDR2
SISSSSGYTSYADSVKG

SEQ ID NO: 139 - protein, 15 aa, synthetic construct
  REGION 1..15: note = X17 HCDR3
GDHGYYVFGYLGMDY

SEQ ID NO: 140 - protein, 17 aa, synthetic construct
  REGION 1..17: note = X18 HCDR3
KYSKRAYPDYYWRGLDY

SEQ ID NO: 141 - protein, 11 aa, synthetic construct
  REGION 1..11: note = X17 LCDR1
RASQSVSSAVA

SEQ ID NO: 142 - protein, 11 aa, synthetic construct
  REGION 1..11: note = X18 LCDR1
RASQSVSSAVA

SEQ ID NO: 143 - protein, 7 aa, synthetic construct
  REGION 1..7: note = X17 LCDR2
SASSLYS

SEQ ID NO: 144 - protein, 7 aa, synthetic construct
  REGION 1..7: note = X18 LCDR2
SASSLYS

SEQ ID NO: 145 - protein, 11 aa, synthetic construct
  REGION 1..11: note = X17 LCDR3
QQSYKPGALIT

SEQ ID NO: 146 - protein, 9 aa, synthetic construct
  REGION 1..9: note = X18 LCDR3
QQSYWWPIT

SEQ ID NO: 147 - protein, 5 aa, synthetic construct
  REGION 1..5: note = 14D10 HCDR1
DYGMS

SEQ ID NO: 148 - protein, 7 aa, synthetic construct
  REGION 1..7: note = 17H10 HCDR1
ARYYSWS

SEQ ID NO: 149 - protein, 5 aa, synthetic construct
  REGION 1..5: note = 16F11 HCDR1
LYYMN

SEQ ID NO: 150 - protein, 17 aa, synthetic construct
  REGION 1..17: note = 14D10 HCDR2
AIPWNGGSTYYKESVKG

SEQ ID NO: 151 - protein, 16 aa, synthetic construct
  REGION 1..16: note = 17H10 HCDR2
VIAYDGSTYYSPSLKS

SEQ ID NO: 152 - protein, 16 aa, synthetic construct
  REGION 1..16: note = 16F11 HCDR2
VIDTHSIAYYADSVKG

SEQ ID NO: 153 - protein, 17 aa, synthetic construct
  REGION 1..17: note = 3B2g1m1 HCDR2
AIPWSGGSTYYKESVKG

SEQ ID NO: 154 - protein, 17 aa, synthetic construct
  REGION 1..17: note = 3B2g1m2 HCDR2
AIPGSGGSTYYKESVKG

SEQ ID NO: 155 - protein, 17 aa, synthetic construct
  REGION 1..17: note = 3B2g1m4 HCDR2
AIPWQGGSTYYKESVKG

SEQ ID NO: 156 - protein, 12 aa, synthetic construct
  REGION 1..12: note = 14D10 HCDR3
RSGRIAFGALDA

SEQ ID NO: 157 - protein, 11 aa, synthetic construct
  REGION 1..11: note = 17H10 HCDR3
GSSRVAAAFDS

SEQ ID NO: 158 - protein, 7 aa, synthetic construct
  REGION 1..7: note = 16F11 HCDR3
GRTALVR

SEQ ID NO: 159 - protein, 14 aa, synthetic construct
  REGION 1..14: note = 14D10 LCDR1
GLSSGSVTSSNYPD

SEQ ID NO: 160 - protein, 14 aa, synthetic construct
  REGION 1..14: note = 3C4 LCDR1
GLSSGSVTASNYPD

SEQ ID NO: 161 - protein, 11 aa, synthetic construct
  REGION 1..11: note = 17H10 LCDR1
GGNRIGGKSVQ

SEQ ID NO: 162 - protein, 11 aa, synthetic construct
  REGION 1..11: note = 23B6 LCDR1
GGDNIGSKNAQ

SEQ ID NO: 163 - protein, 11 aa, synthetic construct
  REGION 1..11: note = 30E1 LCDR1
GGDNIGSKNTQ

SEQ ID NO: 164 - protein, 11 aa, synthetic construct
  REGION 1..11: note = 30A11 LCDR1
GGDNIASKNVQ

SEQ ID NO: 165 - protein, 17 aa, synthetic construct
  REGION 1..17: note = 16F11 LCDR1
KSSQSVVFGSNQKSYLN

SEQ ID NO: 166 - protein, 17 aa, synthetic construct
  REGION 1..17: note = 4C11 LCDR1
RSSQSVLYSSNQKNYLN

SEQ ID NO: 167 - protein, 15 aa, synthetic construct
  REGION 1..15: note = 7A12 LCDR1
ESSQSVLYNQKNYLN

SEQ ID NO: 168 - protein, 17 aa, synthetic construct
  REGION 1..17: note = 7G12 LCDR1
KSSQRVQLGSNQKSYLN

SEQ ID NO: 169 - protein, 15 aa, synthetic construct
  REGION 1..15: note = 7B8 LCDR1
KSSQSVLYNQKNYLA

SEQ ID NO: 170 - protein, 7 aa, synthetic construct
  REGION 1..7: note = 14D10 LCDR2
TTNSRHS

SEQ ID NO: 171 - protein, 7 aa, synthetic construct
  REGION 1..7: note = 7G4 LCDR2
STNSRHS

SEQ ID NO: 172 - protein, 7 aa, synthetic construct
  REGION 1..7: note = 3C4 LCDR2
STDSRHS

SEQ ID NO: 173 - protein, 7 aa, synthetic construct
  REGION 1..7: note = 31G2 LCDR2
STNSRLS

SEQ ID NO: 174 - protein, 7 aa, synthetic construct
  REGION 1..7: note = 17H10 LCDR2
ADSRRPS

SEQ ID NO: 175 - protein, 7 aa, synthetic construct
  REGION 1..7: note = 16F11 LCDR2
YASTQES

SEQ ID NO: 176 - protein, 7 aa, synthetic construct
  REGION 1..7: note = 4C11 LCDR2
WASARES

SEQ ID NO: 177 - protein, 7 aa, synthetic construct
  REGION 1..7: note = 7A12 LCDR2
WASTRQS

SEQ ID NO: 178 - protein, 7 aa, synthetic construct
  REGION 1..7: note = 7G12 LCDR2
YASTQQS

SEQ ID NO: 179 - protein, 7 aa, synthetic construct
  REGION 1..7: note = 7B8 LCDR2
WASTRES

SEQ ID NO: 180 - protein, 12 aa, synthetic construct
  REGION 1..12: note = 14D10 LCDR3
ALYMGGGSNVYV

SEQ ID NO: 181 - protein, 13 aa, synthetic construct
  REGION 1..13: note = 7G4 LCDR3
ALYMGRGSNKDYV

SEQ ID NO: 182 - protein, 12 aa, synthetic construct
  REGION 1..12: note = 3C4 LCDR3
ALYMYSDSKLYV

SEQ ID NO: 183 - protein, 12 aa, synthetic construct
  REGION 1..12: note = 3B2 LCDR3
GLYMYSGSKNYV

SEQ ID NO: 184 - protein, 12 aa, synthetic construct
  REGION 1..12: note = 3G3 LCDR3
ALYMGSDIRNYV

SEQ ID NO: 185 - protein, 12 aa, synthetic construct
  REGION 1..12: note = 31G2 LCDR3
ALYMGSGSRNYV

SEQ ID NO: 186 - protein, 12 aa, synthetic construct
  REGION 1..12: note = 31B7 LCDR3
ALYMGSESRNYV

SEQ ID NO: 187 - protein, 10 aa, synthetic construct
  REGION 1..10: note = 17H10 LCDR3
HVWGSTASAD

SEQ ID NO: 188 - protein, 10 aa, synthetic construct
  REGION 1..10: note = 23B6 LCDR3
HVWDSSTNAW

SEQ ID NO: 189 - protein, 11 aa, synthetic construct
  REGION 1..11: note = 30A11 LCDR3
QVWDSSTNVAV

SEQ ID NO: 190 - protein, 8 aa, synthetic construct
  REGION 1..8: note = 16F11 LCDR3
QQAYSAPT

SEQ ID NO: 191 - protein, 9 aa, synthetic construct
  REGION 1..9: note = 4C11 LCDR3
QQSYKPPYG

SEQ ID NO: 192 - protein, 9 aa, synthetic construct
  REGION 1..9: note = 7A12 LCDR3
QQAYNAPLT

SEQ ID NO: 193 - protein, 9 aa, synthetic construct
  REGION 1..9: note = 7G12 LCDR3
QQGYSAPFT

SEQ ID NO: 194 - protein, 9 aa, synthetic construct
  REGION 1..9: note = 7B8 LCDR3
QQGYSVPYT

SEQ ID NO: 195 - protein, 12 aa, synthetic construct
  REGION 1..12: note = 3B2g2m1 LCDR3
GLYSYSGSKNYV

SEQ ID NO: 196 - protein, 121 aa, synthetic construct
  REGION 1..121: note = 14D10 VH
ELQLVESGGGLVQPGGSLRLSCAASGFTFDDYGMSWVRQAPGKGLEWVSAIPWNGGSTYYKESVKGRFTISRDNAKKTLYLQMNSLKSEDTAVYYCAKRSGRIAFGALDAWGQGTLVTVSS

SEQ ID NO: 197 - protein, 112 aa, synthetic construct
  REGION 1..112: note = VL
QAVVTQEPSLSVSPGGTVTLTCGLSSGSVTSSNYPDWYQQTPGQAPRTLIYTTNSRHSGVPSRFSGSISGNKAALTITGAQPEDEADYYCALYMGGGSNVYVFGGGTKLTVL

SEQ ID NO: 198 - protein, 121 aa, synthetic construct; identical to SEQ ID NO: 196
  REGION 1..121: note = 7G4 VH

SEQ ID NO: 199 - protein, 113 aa, synthetic construct
  REGION 1..113: note = VL
QAVVTQEPSLSVSPGGTVTLTCGLSSGSVTSSNYPDWYQQTPGQAPRALIYSTNSRHSGVPSRFSGSISGNKAALTITGAQPEDEADYYCALYMGRGSNKDYVFGGGTKLTVL

SEQ ID NO: 200 - protein, 121 aa, synthetic construct; identical to SEQ ID NO: 196
  REGION 1..121: note = 3C4 VH

SEQ ID NO: 201 - protein, 112 aa, synthetic construct
  REGION 1..112: note = VL
QAVVTQEPSLSVSPGGTVTLTCGLSSGSVTASNYPDWYQQTPGQAPRGLIYSTDSRHSGVPSRFSGSISGNKAALTITGAQPEDEADYYCALYMYSDSKLYVFGGGTKLTVL

SEQ ID NO: 202 - protein, 121 aa, synthetic construct; identical to SEQ ID NO: 196
  REGION 1..121: note = 3B2 VH

SEQ ID NO: 203 - protein, 112 aa, synthetic construct
  REGION 1..112: note = VL
QAVVTQEPSLSVSPGGTVTLTCGLSSGSVTSSNYPDWYQQTPGQAPRGLIYSTDSRHSGVPSRFSGSISGNKAALTITGAQSEDEADYYCGLYMYSGSKNYVFGGGTKLTVL

SEQ ID NO: 204 - protein, 121 aa, synthetic construct; identical to SEQ ID NO: 196
  REGION 1..121: note = 3G3 VH

SEQ ID NO: 205 - protein, 112 aa, synthetic construct
  REGION 1..112: note = VL
QTVVTQEPSLSVSPGGTVTLTCGLSSGSVTSSNYPDWYQQTPGQAPRALIYSTNSRHSGVPSRFSGSTSGNKAALTITGAQPEDEADYYCALYMGSDIRNYVFGGGTKLTVL

SEQ ID NO: 206 - protein, 121 aa, synthetic construct; identical to SEQ ID NO: 196
  REGION 1..121: note = 31G2 VH

SEQ ID NO: 207 - protein, 112 aa, synthetic construct
  REGION 1..112: note = VL
QAVVTQEPSLSVSPGGTVTLTCGLSSGSVTSSNYPDWYQQTPGQAPRALIYSTNSRLSGVPSRFSGSFSGNKAALTITGAQPEDEADYYCALYMGSGSRNYVFGGGTKLTVL

SEQ ID NO: 208 - protein, 121 aa, synthetic construct; identical to SEQ ID NO: 196
  REGION 1..121: note = 31B7 VH

SEQ ID NO: 209 - protein, 112 aa, synthetic construct
  REGION 1..112: note = VL
QAVVTQEPSLSVSPGGTVTLTCGLSSGSVTSSNYPDWYQQTPGQAPRALIYSTNSRLSGVPSRFSGSFSGNKAALTITGAQPEDEADYYCALYMGSESRNYVFGGGTKLTVL

SEQ ID NO: 210 - protein, 121 aa, synthetic construct
  REGION 1..121: note = 17H10 VH
QVQVQESGPGLVKPSQTLSLTCTVSGGSITARYYSWSWIRQPPGKGLEWMGVIAYDGSTYYSPSLKSRTSISRDTSKNQFSLHLSSVTPDDTAVYYCARGSSRVAAAFDSWGQGTQVTVSS

SEQ ID NO: 211 - protein, 107 aa, synthetic construct
  REGION 1..107: note = VL
SYELTQSPSVSVALRQTAKITCGGNRIGGKSVQWYQQKPGQAPMLVIYADSRRPSGIPERFTGSNSGNTATLTITGAQAEDEADYYCHVWGSTASADFGGGTHLTVL

SEQ ID NO: 212 - protein, 121 aa, synthetic construct; identical to SEQ ID NO: 210
  REGION 1..121: note = 23B6 VH

SEQ ID NO: 213 - protein, 107 aa, synthetic construct
  REGION 1..107: note = VL
SYELTQSPSVSVALRQTAKITCGGDNIGSKNAQWYQQKPGQAPVMVLYADSRRPSGIPERFSGSNSGNTATLTISGAQAEDEADYYCHVWDSSTNAWFGGGTHLTVL

SEQ ID NO: 214 - protein, 121 aa, synthetic construct; identical to SEQ ID NO: 210
  REGION 1..121: note = 30E1 VH

SEQ ID NO: 215 - protein, 107 aa, synthetic construct
  REGION 1..107: note = VL
SYELTQSPSVSVALRRTAKITCGGDNIGSKNTQWYQQKPGQAPVLVIYADSRRPSGIPERFSGSNSGNTATLTISGAQAEDEADYYCHVWDSSTNAWFGGGTHLTVL

SEQ ID NO: 216 - protein, 121 aa, synthetic construct; identical to SEQ ID NO: 210
  REGION 1..121: note = 30A11 VH

SEQ ID NO: 217 - protein, 108 aa, synthetic construct
  REGION 1..108: note = VL
SYELTQSPSVTVALRQTAKITCGGDNIASKNVQWYQQKPGQAPSLVIWADSRRPSGIPVRFSGSNFGNTATLTISGAQAEDEADYYCQVWDSSTNVAVFGGGTHLTVL

SEQ ID NO: 218 - protein, 115 aa, synthetic construct
  REGION 1..115: note = 16F11 VH
EVQLVESGGGLVQPGGSLSLSCVASGFTFSLYYMNWVRQAPGKGLEWLSVIDTHSIAYYADSVKGRFTISRDNVKNTLYLQLNNLKPEDTALYYCVLGRTALVRWGQGTQVTVSS

SEQ ID NO: 219 - protein, 112 aa, synthetic construct
  REGION 1..112: note = VL
DIVMTQSPSSVTASVGEKVTINCKSSQSVVFGSNQKSYLNWYQQRPGQSPRLLIYYASTQESGIPDRFSGSGSTTDFTLTISSVQPEDAAVYYCQQAYSAPTFGSGTRLEIK

SEQ ID NO: 220 - protein, 115 aa, synthetic construct; identical to SEQ ID NO: 218
  REGION 1..115: note = 4C11 VH

SEQ ID NO: 221 - protein, 113 aa, synthetic construct
  REGION 1..113: note = VL
DIVMTQSPSSVTASAGERVTINCRSSQSVLYSSNQKNYLNWYQQRLGQSPRLLIYWASARESGVPDRFSGSGSTTNFTLTISSFQPEDAAVYYCQQSYKPPYGFGSGTRLEIK

SEQ ID NO: 222 - protein, 115 aa, synthetic construct; identical to SEQ ID NO: 218
  REGION 1..115: note = 7A12 VH

SEQ ID NO: 223 - protein, 113 aa, synthetic construct
  REGION 1..113: note = VL
EIVLTQSPSSVTASIGEKVTINCESSQSVLYNQKNYLNWYQQRPGQSPRLLIYWASTRQSGVPDRFSGSGSGSTTDFTLTISSFQPEDVAVYYCQQAYNAPLTFGPGTKVELK

SEQ ID NO: 224 - protein, 115 aa, synthetic construct; identical to SEQ ID NO: 218
  REGION 1..115: note = 7G12 VH

SEQ ID NO: 225 - protein, 113 aa, synthetic construct
  REGION 1..113: note = VL
EIVLTQSPNSVTASVGEKVTINCKSSQRVQLGSNQKSYLNWYQQRPGQSPRLLIYYASTQQSGIPDRFSGSGSATDFTLTINSVQPEDAAVYYCQQGYSAPFTFGQGTKVELK

SEQ ID NO: 226 - protein, 115 aa, synthetic construct; identical to SEQ ID NO: 218
  REGION 1..115: note = 7B8 VH

SEQ ID NO: 227 - protein, 111 aa, synthetic construct
  REGION 1..111: note = VL
EIVLTQSPSSVTASAGEKVTINCKSSQSVLYNQKNYLAWYQQRPGQSPRLLIYWASTRESGVPDRFSGSGSTTDFTLTISSFQPEDVAVYYCQQGYSVPYTFGSGTRLEIK

SEQ ID NO: 228 - protein, 121 aa, synthetic construct
  REGION 1..121: note = 3B2g1m1 VH
EVQLLESGGGLVQPGGSLRLSCAASGFTFSDYGMSWVRQAPGKGLEWVSAIPWSGGSTYYKESVKGRFTISRDNSKNTLYLQMNSLRAEDTAVYYCAKRSGRIAFGALDAWGQGTLVTVSS

SEQ ID NO: 229 - protein, 112 aa, synthetic construct
  REGION 1..112: note = VL
QTVVTQEPSFSVSPGGTVTLTCGLSSGSVTSSNYPDWYQQTPGQAPRTLIYSTDSRHSGVPDRFSGSILGNKAALTITGAQADDESDYYCGLYMYSGSKNYVFGGGTKLTVL

SEQ ID NO: 230 - protein, 121 aa, synthetic construct
  REGION 1..121: note = 3B2g1m2 VH
EVQLLESGGGLVQPGGSLRLSCAASGFTFSDYGMSWVRQAPGKGLEWVSAIPGSGGSTYYKESVKGRFTISRDNSKNTLYLQMNSLRAEDTAVYYCAKRSGRIAFGALDAWGQGTLVTVSS

SEQ ID NO: 231 - protein, 112 aa, synthetic construct; identical to SEQ ID NO: 229
  REGION 1..112: note = VL

SEQ ID NO: 232 - protein, 121 aa, synthetic construct
  REGION 1..121: note = 3B2g1m4 VH
EVQLLESGGGLVQPGGSLRLSCAASGFTFSDYGMSWVRQAPGKGLEWVSAIPWQGGSTYYKESVKGRFTISRDNSKNTLYLQMNSLRAEDTAVYYCAKRSGRIAFGALDAWGQGTLVTVSS

SEQ ID NO: 233 - protein, 112 aa, synthetic construct; identical to SEQ ID NO: 229
  REGION 1..112: note = VL

SEQ ID NO: 234 - protein, 121 aa, synthetic construct; identical to SEQ ID NO: 228
  REGION 1..121: note = 3B2g2m1 VH

SEQ ID NO: 235 - protein, 112 aa, synthetic construct
  REGION 1..112: note = VL
QTVVTQEPSFSVSPGGTVTLTCGLSSGSVTSSNYPDWYQQTPGQAPRTLIYSTDSRHSGVPDRFSGSILGNKAALTITGAQADDESDYYCGLYSYSGSKNYVFGGGTKLTVL

SEQ ID NO: 236 - protein, 121 aa, synthetic construct; identical to SEQ ID NO: 230
  REGION 1..121: note = 3B2g2m2 VH

SEQ ID NO: 237 - protein, 112 aa, synthetic construct; identical to SEQ ID NO: 235
  REGION 1..112: note = VL

SEQ ID NO: 238 - protein, 121 aa, synthetic construct; identical to SEQ ID NO: 232
  REGION 1..121: note = 3B2g2m4 VH

SEQ ID NO: 239 - protein, 112 aa, synthetic construct; identical to SEQ ID NO: 235
  REGION 1..112: note = VL

SEQ ID NO: 240 - protein, 15 aa, synthetic construct
  REGION 1..15: note = X2m1 HCDR3
SEGDRYVSGYFGFDY

SEQ ID NO: 241 - protein, 15 aa, synthetic construct
  REGION 1..15: note = X2m2 HCDR3
SEGDRYVSGYFGLDY

SEQ ID NO: 242 - protein, 15 aa, synthetic construct
  REGION 1..15: note = X2m3 HCDR3
SEGDRYVSGYSGFDY

SEQ ID NO: 243 - protein, 15 aa, synthetic construct
  REGION 1..15: note = X2m4 HCDR3
SEGDRYVSGYSGLDY

SEQ ID NO: 244 - protein, 15 aa, synthetic construct
  REGION 1..15: note = X2m5 HCDR3
SEGDRYVSGYFGMDY

SEQ ID NO: 245 - protein, 15 aa, synthetic construct
  REGION 1..15: note = X2m6 HCDR3
SEGDRYVSGYSGMDY

SEQ ID NO: 246 - protein, 15 aa, synthetic construct
  REGION 1..15: note = X2m7 HCDR3
SEGDRYVSGYMGFDY

SEQ ID NO: 247 - protein, 15 aa, synthetic construct
  REGION 1..15: note = X2m8 HCDR3
SEGDRYVSGYMGLDY

SEQ ID NO: 248 - protein, 15 aa, synthetic construct
  REGION 1..15: note = X17m1 HCDR3
GDHGYYVSGYLGMDY

SEQ ID NO: 249 - protein, 15 aa, synthetic construct
  REGION 1..15: note = X17m2 HCDR3
GDHGYYVYGYLGMDY

SEQ ID NO: 250 - protein, 15 aa, synthetic construct
  REGION 1..15: note = X17m3 HCDR3
GDHGYYVSGYLGFDY

SEQ ID NO: 251 - protein, 15 aa, synthetic construct
  REGION 1..15: note = X17m6 HCDR3
GEHGYYVSGYLGFDY

SEQ ID NO: 252 - protein, 124 aa, synthetic construct
  REGION 1..124: note = X2m1 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYFGFDYWGQGTLVTVSS

SEQ ID NO: 253 - protein, 124 aa, synthetic construct
  REGION 1..124: note = X2m2 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYFGLDYWGQGTLVTVSS

SEQ ID NO: 254 - protein, 124 aa, synthetic construct
  REGION 1..124: note = X2m3 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYSGFDYWGQGTLVTVSS

SEQ ID NO: 255 - protein, 124 aa, synthetic construct
  REGION 1..124: note = X2m4 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYSGLDYWGQGTLVTVSS

SEQ ID NO: 256 - protein, 124 aa, synthetic construct
  REGION 1..124: note = X2m5 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYFGMDYWGQGTLVTVSS

SEQ ID NO: 257 - protein, 124 aa, synthetic construct
  REGION 1..124: note = X2m6 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYSGMDYWGQGTLVTVSS

SEQ ID NO: 258 - protein, 124 aa, synthetic construct
  REGION 1..124: note = X2m7 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYMGFDYWGQGTLVTVSS

SEQ ID NO: 259 - protein, 124 aa, synthetic construct
  REGION 1..124: note = X2m8 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYMGLDYWGQGTLVTVSS

SEQ ID NO: 260 - protein, 124 aa, synthetic construct
  REGION 1..124: note = X17m1 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVSGYLGMDYWGQGTLVTVSS

SEQ ID NO: 261 - protein, 124 aa, synthetic construct
  REGION 1..124: note = X17m2 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVYGYLGMDYWGQGTLVTVSS

SEQ ID NO: 262 - protein, 124 aa, synthetic construct
  REGION 1..124: note = X17m3 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVSGYLGFDYWGQGTLVTVSS

SEQ ID NO: 263 - protein, 124 aa, synthetic construct
  REGION 1..124: note = X17m6 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGEHGYYVSGYLGFDYWGQGTLVTVSS

SEQ ID NO: 264 - DNA, 1432 bp, synthetic construct
  misc_feature 1..1432: note = VH_3B2g2m1 coding sequence
  source 1..1432: mol_type = other DNA
aagcttgccgccaccatggaatggtcctgggtgttcctgttcttcctgtccgtgaccaccggcgtgcactctgaagttcagctgctggaatctggcggcggattggttcaacctggcggctctctgagactgtcttgtgccgcttctggcttcaccttctccgactacggcatgtcctgggtccgacaggctcctggcaaaggactggaatgggtgtccgccattccttggagcggcggctccacctactacaaagaatccgtgaagggccgctttaccatcagccgggacaactccaagaacaccctgtacctgcagatgaactccctgagagccgaggacaccgccgtgtactactgcgctaagagatccggcagaatcgccttcggcgctctggatgcttggggacagggaacactggtcaccgtgtcctccgcttctaccaagggaccctctgtgttccctctggctccttccagcaagtctacctctggcggaacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattctttctgtactccaagctgacagtggacaagtccagatggcagcagggcaacgtgttctcctgcagcgtgatgcacgaggccctgcacaatcactacacacagaagtccctgtctctgtcccctggctgatgaattc

SEQ ID NO: 265 - DNA, 739 bp, synthetic construct
  misc_feature 1..739: note = VL_3B2g2m1 coding sequence
  source 1..739: mol_type = other DNA
aagcttgccgccaccatgtctgtgcctacacaggttctgggactgctgctgctgtggctgaccgatgctagatgccagaccgtcgtgacccaagagccttccttctctgtgtctcctggcggcaccgtgacactgacatgtggactgtcctctggctccgtgacctccagcaactaccccgactggtatcagcagacccctggacaggctcctcggacactgatctactccaccgactccagacactctggcgtgcccgatagattctccggctctatcctgggcaacaaggccgctctgacaatcactggcgcccaggccgatgacgagtccgattactactgcggcctgtactcctactccggctccaagaactacgtgttcggcggaggcaccaagctgacagtgctgggacaacctaaggctgccccttccgtgactctgttccctccatcctctgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctcctgatgaattc

SEQ ID NO: 266 - protein, 220 aa, synthetic construct
  REGION 1..220: note = Fcc with K (human domain)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 219 aa, synthetic construct
  REGION 1..219: note = Fcc without K (human domain)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 268 - protein, 450 aa, synthetic construct
  REGION 1..450: note = ARGX-119 hIgG1, full length heavy chain, reduced effector function, delK
EVQLLESGGGLVQPGGSLRLSCAASGFTFSDYGMSWVRQAPGKGLEWVSAIPWSGGSTYYKESVKGRFTISRDNSKNTLYLQMNSLRAEDTAVYYCAKRSGRIAFGALDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 269 - protein, 218 aa, synthetic construct
  REGION 1..218: note = ARGX-119 hIgG1, full length light chain, reduced effector function, delK
QTVVTQEPSFSVSPGGTVTLTCGLSSGSVTSSNYPDWYQQTPGQAPRTLIYSTDSRHSGVPDRFSGSILGNKAALTITGAQADDESDYYCGLYSYSGSKNYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 270 - protein, 451 aa, synthetic construct
  REGION 1..451: note = ARGX-119 hIgG1, full length heavy chain, reduced effector function
EVQLLESGGGLVQPGGSLRLSCAASGFTFSDYGMSWVRQAPGKGLEWVSAIPWSGGSTYYKESVKGRFTISRDNSKNTLYLQMNSLRAEDTAVYYCAKRSGRIAFGALDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 218 aa, synthetic construct; identical to SEQ ID NO: 269
  REGION 1..218: note = ARGX-119 hIgG1, full length light chain, reduced effector function

SEQ ID NO: 272 - protein, 449 aa, synthetic construct
  REGION 1..449: note = Mota-hIgG1, full length heavy chain, reduced effector function, delK
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 273 - protein, 213 aa, synthetic construct
  REGION 1..213: note = Mota-hIgG1, full length light chain, reduced effector function, delK
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 450 aa, synthetic construct
  REGION 1..450: note = Mota-hIgG1, full length heavy chain, reduced effector function
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 275 - protein, 213 aa, synthetic construct; identical to SEQ ID NO: 273
  REGION 1..213: note = Mota-hIgG1, full length light chain, reduced effector function

SEQ ID NO: 276 - DNA, 1350 bp, synthetic construct
  misc_feature 1..1350: note = coding DNA for HC_ARGX-119
  source 1..1350: mol_type = other DNA
gaagttcagctgctggaatctggcggcggattggttcaacctggcggctctctgagactgtcttgtgccgcttctggcttcaccttctccgactacggcatgtcctgggtccgacaggctcctggcaaaggactggaatgggtgtccgccattccttggagcggcggctccacctactacaaagaatccgtgaagggccgctttaccatcagccgggacaactccaagaacaccctgtacctgcagatgaactccctgagagccgaggacaccgccgtgtactactgcgctaagagatccggcagaatcgccttcggcgctctggatgcttggggacagggaacactggtcaccgtgtcctccgcttctaccaagggaccctctgtgttccctctggctccttccagcaagtctacctctggcggaacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattctttctgtactccaagctgacagtggacaagtccagatggcagcagggcaacgtgttctcctgcagcgtgatgcacgaggccctgcacaatcactacacacagaagtccctgtctctgtcccctggc

SEQ ID NO: 277 - DNA, 363 bp, synthetic construct
  misc_feature 1..363: note = coding DNA for VH_ARGX-119
  source 1..363: mol_type = other DNA
gaagttcagctgctggaatctggcggcggattggttcaacctggcggctctctgagactgtcttgtgccgcttctggcttcaccttctccgactacggcatgtcctgggtccgacaggctcctggcaaaggactggaatgggtgtccgccattccttggagcggcggctccacctactacaaagaatccgtgaagggccgctttaccatcagccgggacaactccaagaacaccctgtacctgcagatgaactccctgagagccgaggacaccgccgtgtactactgcgctaagagatccggcagaatcgccttcggcgctctggatgcttggggacagggaacactggtcaccgtgtcctcc

SEQ ID NO: 278 - DNA, 654 bp, synthetic construct
  misc_feature 1..654: note = coding DNA for LC_ARGX-119
  source 1..654: mol_type = other DNA
cagaccgtcgtgacccaagagccttccttctctgtgtctcctggcggcaccgtgacactgacatgtggactgtcctctggctccgtgacctccagcaactaccccgactggtatcagcagacccctggacaggctcctcggacactgatctactccaccgactccagacactctggcgtgcccgatagattctccggctctatcctgggcaacaaggccgctctgacaatcactggcgcccaggccgatgacgagtccgattactactgcggcctgtactcctactccggctccaagaactacgtgttcggcggaggcaccaagctgacagtgctgggacaacctaaggctgccccttccgtgactctgttccctccatcctctgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctcc

SEQ ID NO: 279 - DNA, 336 bp, synthetic construct
  misc_feature 1..336: note = coding DNA for VL_ARGX-119
  source 1..336: mol_type = other DNA
cagaccgtcgtgacccaagagccttccttctctgtgtctcctggcggcaccgtgacactgacatgtggactgtcctctggctccgtgacctccagcaactaccccgactggtatcagcagacccctggacaggctcctcggacactgatctactccaccgactccagacactctggcgtgcccgatagattctccggctctatcctgggcaacaaggccgctctgacaatcactggcgcccaggccgatgacgagtccgattactactgcggcctgtactcctactccggctccaagaactacgtgttcggcggaggcaccaagctgacagtgctg